(19) United States
(12) Patent Application Publication
(10) Pub. No.: US 2003/0073110 A1
(43) Pub. Date: Apr. 17, 2003

(54) Method for isolating nucleic acid and a cartridge for chemical reaction and for nucleic acid isolation

(76) Inventors: Masaharu Aritomi, Shizuoka (JP); Akiko Sato, Shizuoka (JP)

Correspondence Address: BIRCH STEWART KOLASCH & BIRCH, PO BOX 747, FALLS CHURCH, VA, 22040-0747, US

(21) Appl. No.: 10/188,059

(22) Filed: Jul. 3, 2002

(30) Foreign Application Priority Data
Jul. 3, 2001 (JP) 202502/2001
Oct. 11, 2001 (JP) 313511/2001
Dec. 26, 2001 (JP) 393445/2001
Jun. 28, 2002 (JP) 189729/2002

(51) Int. Cl. C12Q 1/68; C07H 21/04

(52) U.S. Cl. 435/6; 536/25.4

(57) ABSTRACT

The present invention provides a method for isolating nucleic acid comprising a step of preparing suspension containing nucleic acid-adsorbed nucleic acid-binding carriers by mixing material containing nucleic acid, nucleic acid-binding carriers and a solution for adsorbing/releasing nucleic acid, wherein the step is conducted under heating, a step of separating nucleic acid-adsorbed nucleic acid-binding carriers from a liquid phase, a step of washing nucleic acid-adsorbed nucleic acid-binding carriers, a step of drying and a step of eluting nucleic acid, and a cartridge for chemical reaction that enables such chemical reaction to be performed quickly and conveniently.

### FIELD OF THE INVENTION

The present invention relates to a method for isolating nucleic acid simply and rapidly from material containing nucleic acid. The present invention also relates to a method for applying an amplification reaction after effectively isolating a trace amount of nucleic acid. The present invention further relates to a cartridge for chemical reaction having a novel structure. Further, the present invention relates to a cartridge for chemical reaction for carrying out a novel method for feeding liquid. The present invention also relates to a flow path for nucleic acid isolation that applies the above method for isolating nucleic acid. The present invention further relates to a cartridge for nucleic acid isolation that applies the structure of the above cartridge for chemical reaction.

### BACKGROUND OF THE INVENTION

Accompanying advances in the field of genetic engineering, clinical diagnosis and microorganism testing using genes are now being performed. For example, using a nucleic acid hybridization technique, a test procedure that performs identification of a bacterial strain of a microorganism has come into practical use. Further, the development of various methods of nucleic acid amplification, represented by the polymerase chain reaction method (PCR method, Science, 230:1350-1354, 1985; Japanese Patent Nos. 2546576, 2502041, and 2613877), has greatly expanded the range of clinical diagnosis and microorganism testing using genes. These techniques allow nucleic acid amplification to be performed sequence specifically. As a result, it is possible to amplify a specific nucleic acid which may possibly be present in a specimen, and detect it at a high sensitivity.

However, if a contaminating substance such as a protein, lipid, or carbohydrate is contained in a large amount in a specimen used in clinical diagnosis or microorganism testing, the contaminating substance will exert an adverse effect when conducting an amplification reaction, such as a polymerase chain reaction, or a hybridization reaction. For example, in a case where an amplification reaction includes an inhibiting factor, in practice, even if a bacteria of interest is contained in a specimen, since an amplification reaction does not occur, an incorrect result that the bacteria is not contained in the specimen is given. Therefore, in order not to exert an influence on a detection reaction such as a nucleic acid amplification reaction or hybridization, an operation to isolate nucleic acid in the specimen beforehand is necessary.

Methods for isolating a nucleic acid include a method described in Japanese Patent No. 2680462 (U.S. Pat. No. 5,234,809). This method is executed by adding a high-chaotropic aqueous solution, such as a guanidine thiocyanate solution, to a material containing nucleic acid to release the nucleic acid, and then allowing the nucleic acid to adsorb on silica particles, washing the silica particles to separate contaminants, and finally eluting the nucleic acid adsorbed to the silica particles. A reagent and tools that perform isolation of nucleic acid based on this method are commercially available as a so-called “kit.”

However, when a high-chaotropic substance, such as guanidine thiocyanate, that is used at the time of release, adsorption or washing, or an organic solvent or the like such as ethanol remains in a nucleic acid solution isolated by the above technique, the problem frequently occurs that these substances inhibit a polymerase chain reaction. For example, the instructions attached to the Mag Exractor™ (Genome) kit of Toyobo Co., Ltd. state that “when the amount of extracted nucleic acid solution present in a polymerase chain reaction system exceeds ⅕ of the volume thereof, there is a possibility that the reaction will be inhibited.” When such kind of inhibition occurs, even though a nucleic acid of interest is actually contained in the solution, because an amplification reaction does not occur, ultimately the amplification product can not be verified, and the mistaken result that the solution does not contain the nucleic acid is given.

To overcome these problems, in the above method it is necessary to apply methods such as, 1. a method that sufficiently separates washing liquid by a centrifugation operation at a stage when nucleic acid has adsorbed to silica particles; 2. a method that removes washing liquids by drying, by opening the cover of a container and heating; 3. a method that removes inhibitors from the nucleic acid solution by dialysis or the like; and 4. a method in which eluted nucleic acid solution is diluted to a degree that does not cause inhibition of the polymerase chain reaction or hybridization reaction. However, the centrifugation operation of the method of 1. is complicated and requires time and labor. The method of 2. in which drying is performed by opening the cover of a container increases the risk of contamination with other microorganisms or nucleic acid present in the atmosphere. In practice, when performing a polymerase chain reaction, contamination with nucleic acid from the surrounding environment is frequently the cause of a false positive result in laboratory tests, thus constituting a problem. The dialysis operation or the like of 3. is problematic in that it is complicated and requires time until a result is obtained. The method of 4. in which inhibition is avoided by dilution, means that detection at high sensitivity which is an advantage of the amplification technique is lost, and creates the possibility that nucleic acid present in a trace amount will not be detected. For the above reasons, in a process to isolate nucleic acid, the quantity of the above inhibitors present significantly influences the sensitivity of the ultimate detection. Accordingly, there is a need for development of a simple and convenient method for preparing nucleic acid solution that does not contain the above inhibitors.

On the other hand, at the actual site of clinical examination for medical treatment, a diagnostic method is sought in which a specimen collected from a patient is tested quickly and conveniently at the bedside, and the result is determined and immediately utilized. This type of diagnostic method is known as a so-called “point of care testing.” Hereinafter these tests are referred to as “POCT.” It is required that these tests be performed in as short a time as possible and in a simple manner that involves little manual operation. Specifically, it is desirable that the steps from treatment of the specimen to detection are completed inside one apparatus, which is called a “cartridge.” In the case of diagnosis using nucleic acid, it is desirable that the steps of isolation of nucleic acid and the subsequently performed amplification or, furthermore, detection can be completed inside one “cartridge.” Moreover, it is desirable that the cartridge is designed such that it can be manufactured at a low cost and is “disposable” or easily “recyclable.”

As microfabrication technology has evolved, technology has been developed that can complete such kind of clinical diagnosis and the like in a cartridge. Studies have proceeded that apply the technology of the “micro total analysis system (μTAS),” in which conventionally utilized analyzers are miniturized and liquid reagents are reacted to trace amounts, to POCT. In μTAS, in order to make a specimen amount into a trace amount, a groove is carved on the surface of glass or silicon, a reagent solution or specimen is poured into the groove, isolation and reaction are performed, and analysis of a trace amount of the sample is conducted (Japanese Patent Application Laying-Open (kokai) No. 2-245655, Japanese Patent Application Laying-Open (kokai) No. 3-226666, Japanese Patent Application Laying-Open (kokai) No. 8-233778, Analytical Chem. 69, 2626-2630, (1997) Aclara Biosciences, and the like). The present applicants have also submitted patent applications for inventions relating to μTAS, including “Analyzer,” WO99-64846, and “Analyzing Cartridge and Liquid Feed Control Device,” WO01-13127. In the specifications of these applications, the use of a resinous microchip as a cartridge is described.

However, in manufacturing these cartridges, it is necessary that the components be glued together such that a flow path, which is fabricated in minute detail, is not blocked. Advanced technology is required for the gluing operation, and it is one of the factors that raise the cost of the cartridge. Moreover, in the case of a cartridge manufactured by a gluing operation using an adhesive, forming one or more valves inside the cartridge is difficult. Further, in the case when an adhesive is used for a gluing operation and fabrication, because such kind of adhesion is generally irreversible, once a cartridge has been fabricated it cannot be disassembled. As a result, reuse of the cartridge is practically impossible. Accordingly, there is a need for a cartridge that can be manufactured at a lower cost, easily, and which has a structure that allows the formation of a valve structure.

Further, conducting a nucleic acid isolation reaction mechanically has come into practical use. In the machine, as a substance that adsorbs nucleic acid mainly in the presence of a high-chaotropic substance, microparticles consisting of magnetic body particles covered by a silica substrate for which separation from a liquid phase can easily be performed using a magnetic field are used (Japanese Patent Application Laying-Open (kokai) No. 9-19292). In the method using these magnetic silica particles, it is essential that the magnetic silica particles be suspended in liquid phase in each step. To ensure this, a physical method for dispersing the magnetic silica particles is necessary. Specific methods of dispersion include a method in which a container is subjected to vibration using a vibratory apparatus, such as a vortex mixer or the like, and a method using a instrument called a pipette, in which fluid inside the container is taken in and out in an intense manner. Further, enhancement of a washing effect by automatic vibration of a magnet is also performed by machine (Japanese Patent Application Laying-Open (kokai) No. 11-215978). However, as that operation is complicated, even though it can be accomplished by large-sized machines, it is extremely difficult to accomplish using the structure of the cartridge required in the above POCT. That is, while machines for isolating nucleic acid that have come into practical use are useful when treating a large number of specimens in a large amount, use of such machines in “isolating a nucleic acid and, further, performing the subsequent amplification reaction and detection reaction rapidly inside a cartridge,” which is needed in POCT, is difficult, and requires an extremely large number of problems to be solved. Accordingly, in diagnosis using nucleic acid, there is no system in practical use that uses a cartridge for this kind of POCT.

As described in the foregoing, in the current situation of POCT using nucleic acid there is a need for, 1. development of a method for isolating nucleic acid that does not include a substance that inhibits an amplification reaction or the like when performing isolation of nucleic acid; 2. development of a cartridge that is simple and enables fabrication of a valve structure, which is suitable for POCT; 3. development of a technique that effectively performs nucleic acid isolation in a cartridge; and 4. development of an apparatus that performs a complete process from nucleic acid isolation to amplification reaction in a cartridge.

### SUMMARY OF THE INVENTION

The present invention succeeds in solving the problems described above. More specifically, an object of the present invention is to provide a method for isolating nucleic acid, in which a substance that inhibits a nucleic acid amplification reaction is not included in a nucleic acid solution isolated from a material containing nucleic acid. Another object of the present invention is to provide a method for isolating nucleic acid from a material containing nucleic acid, simply, effectively, and rapidly, and by means allowing automatic mechanization. A further object of the present invention is to provide an automated device that isolates a nucleic acid using a cartridge. A still further object of the present invention is to provide a method for feeding liquid that is suitable for chemical reaction in a cartridge. A still further object of the present invention is to provide an automatic apparatus for detecting nucleic acid that, after isolation of nucleic acid, completely performs amplification reaction and detection of amplified nucleic acid.

In order to achieve the above objects, we conducted concentrated studies and, as a result, succeeded in completing the present invention.

That is, in one aspect the present invention relates to

(1): a method for isolating nucleic acid, comprising the steps of:

1. mixing a material containing nucleic acid and a solution for adsorbing/releasing nucleic acid, and contacting a mixed solution thereof with nucleic acid-binding carriers to prepare nucleic acid-adsorbed nucleic acid-binding carriers;

2. separating the nucleic acid-adsorbed nucleic acid-binding carriers;

3. washing the nucleic acid-adsorbed nucleic acid-binding carriers with washing liquid;

4. drying the nucleic acid-adsorbed nucleic acid-binding carriers; and

5. eluting nucleic acid from the nucleic acid-binding carriers with eluate,

wherein the above step 1 is conducted under heating.

In another aspect, the present invention relates to

(2): a method for isolating nucleic acid, comprising the steps of:

1. mixing a material containing nucleic acid, nucleic acid-binding magnetic carriers and a solution for adsorbing/releasing nucleic acid to prepare a suspension containing the carriers to which nucleic acid has bound;

2. separating the carriers to which nucleic acid has adsorbed from a liquid phase of the suspension;

3. washing the carriers to which nucleic acid has bound with washing liquid;

4. drying the carriers to which nucleic acid has bound; and

5. eluting nucleic acid from the carriers with eluate,

wherein step 1 is conducted under heating and, in steps 2 to 4, a flow path for nucleic acid isolation is provided along which a magnetic field capable of retaining the carriers can be applied in at least two places, and when suspension containing the carriers to which nucleic acid has bound is flowed in the flow path, the magnetic field is applied at one place of the at least two places and the carriers are separated from the suspension, and when at least one solution for washing is flowed in the flow path, application of the magnetic field at a place where the carriers are retained is released, and by applying the magnetic field at a place downstream of the place the carriers were retained, the carriers are washed and separated from the solution for washing, and further, a solution for eluting nucleic acid is flowed in the flow path to elute nucleic acid from the carriers.

In a further aspect, the present invention relates to

(3): the method for isolating nucleic acid of (1) or (2), wherein heating of step 1 is conducted at a temperature of 60° C. or higher and 130° C. or lower;

(4): the method for isolating nucleic acid of (1) or (2), wherein heating of step 1 is conducted at a temperature of 80° C. or higher and 110° C. or lower;

(5): the method for isolating nucleic acid of (1) or (2), wherein heating of step 1 is conducted at a temperature of 90° C. or higher and 100° C. or lower; and

(6): the method for isolating nucleic acid of (1) or (2), wherein heating of step 1 is conducted for 1 minute or more and 1 hour or less.

In step 1 of the method of the present invention, a material containing nucleic acid and a solution for adsorbing/releasing nucleic acid are mixed and contacted with nucleic acid-binding carriers, to thereby prepare nucleic acid-adsorbed nucleic acid-binding carriers. When using particulate matter such as silica particles mentioned below as a nucleic acid-binding carrier, this step is a step of mixing together the above material containing nucleic acid, nucleic acid-binding carriers and solution for adsorbing/releasing nucleic acid, to prepare a suspension containing nucleic acid-adsorbed nucleic acid-binding carriers. Regarding the ratio of material containing nucleic acid, nucleic acid-binding carriers and solution for adsorbing/releasing nucleic acid, for example, a preferred result can be obtained with a ratio of 10:1:90, however the ratio is not necessarily limited to this. In the case of using a membranous material, such as a silica membrane mentioned below, since it is preferable to first immobilize a membranous nucleic acid-binding carrier in a reaction chamber of the cartridge configuration, in this step, after mixing material containing nucleic acid and solution for adsorbing/releasing nucleic acid, contact the mixed solution with the immobilized nucleic acid-binding carrier. Specifically, configure such that the mixed solution of the material containing nucleic acid and the solution for adsorbing/releasing nucleic acid passes through the membranous nucleic acid-binding carrier, and nucleic acid may thereby be adsorbed to the nucleic acid-binding carrier.

According to the inventions of (1)-(6), it is possible to prevent a factor imparting an inhibiting effect to an amplification reaction from contaminating in an isolated nucleic acid solution. Specifically, there can be provided a method for isolating nucleic acid that does not include substances inhibiting a nucleic acid amplification method, without conducting an operation such as dialysis or a diluting operation.

Since, as described above, in the method of the present invention, nucleic acid is adsorbed to a nucleic acid-binding carrier under heating, isolation of nucleic acid from a material and adsorption of the nucleic acid to a carrier can proceed in an adequate manner without conducting treatment with enzymes such as protease K or lysozyme, which, depending on the selection of material containing nucleic acid, conventionally is often necessary due to bacteriolysis and the like. In the method of the present invention, in the case where heating of the above step 1 is below 60° C., there is a possibility that an amplification reaction does not take place due to contamination of a substance inhibiting a polymerase chain reaction. On the other hand, heating over 130° C. is not preferred since it requires a hermetic container that is resistant to pressurization, and furthermore, because there is a possibility of the nucleic acid being minutely fragmented. A heating time may be 1 minute or more, such that the temperature in the reaction solution rises sufficiently within the above temperature range. Moreover, a heating time exceeding I hour is not preferred since it prevents rapid isolation. A preferred heating time is 5-15 minutes.

In step 2, nucleic acid-adsorbed nucleic acid-binding carriers are separated from suspension obtained in the above step 1 or from a mixed solution of material containing nucleic acid and solution for adsorbing/releasing nucleic acid. As a separation means, a method that is normally used in the art, such as centrifugal separation, can be suitably used. Moreover, when using magnetic silica particles or magnetic silica derivative particles as nucleic acid-binding carriers, the carriers can be simply collected by utilizing a magnet. When using membranous carriers, using an appropriate fluid feeding means or the like, the membranous carriers and the mixed solution of material containing nucleic acid and solution for adsorbing/releasing nucleic acid may be physically separated.

In step 3, nucleic acid-adsorbed nucleic acid-binding carriers, which were separated in step 2, are washed. This washing step is performed by suspending the nucleic acid-adsorbed nucleic acid-binding carriers in a solution for washing, and after optionally performing stirring or the like, using a similar means to that used in the above separation step (step 2), recovery is performed utilizing centrifugal or magnetical separation. This washing step can be executed once or a plurality of times. According to this step 3, a contaminant included in a material containing nucleic acid or a high-chaotropic substance included in a solution for adsorbing/releasing nucleic acid can be physically removed, and, in the case of performing the heating of step 1, which is a feature of the present invention, this step can be performed to particular effect.

Further, when magnetic carriers are accumulated using a magnetic field, in many cases a rigid aggregation is caused that forms a mass. For this type of aggregate, when a washing operation is performed by washing liquid in that state without releasing application of the magnetic field, because the carriers do not disperse, washing liquid does not reach inside the mass of the carriers, and thus separation and removal of contaminants is incomplete. According to the invention of the above (2), since the carriers are dispersed in washing liquid at the time of washing, it is possible for washing liquid to effectively wash the carriers, and thus contaminants other than nucleic acid can be effectively eliminated from nucleic acid-binding carriers. Moreover, when washing or the like of a mass in which the carriers have aggregated due to application of a magnetic field is performed while the carriers are still aggregated, the carriers aggregate more strongly and cause a blockage in the flow path. According to this invention, since the aggregate of carriers is dispersed at the time of washing, the possibility of causing a blockage is low. Further, according to this invention, it is possible to execute extraction and isolation of nucleic acid from material containing the nucleic acid using a nucleic acid-binding magnetic carrier in one series of operations in a flow path for nucleic acid isolation, and thus execution of nucleic acid isolation is simplified.

While the procedure in the above step 2 and step 3 is not particularly limited, after the heating of step 1, a preferred result can be obtained when execution is continued without any specific cooling.

In step 4 of the method of the present invention, a nucleic acid-adsorbed nucleic acid-binding carrier, having been washed, is dried. By drying once, a factor used in washing that may impart an inhibitory effect on a subsequent amplification reaction, such as ethanol or the like, can be removed.

In step 5 of the method of the present invention, nucleic acid is eluted from a nucleic acid-binding carrier. Elution can be performed using water or a buffer solution for elution in a manner normally performed in the art. Moreover, elution can also be performed using a solution containing an enzyme for nucleic acid amplification reaction, an oligonucleotide, and a substrate, to be used in a subsequently performed operation such as, for example, a polymerase chain reaction.

In a further aspect, the present invention relates to

(7): the method for isolating nucleic acid of (1) to (6), wherein the solution for adsorbing/releasing nucleic acid is a solution containing a high-chaotropic substance;

(8): the method for isolating nucleic acid of (1), wherein the carrier is silica or a silica derivative;

(9): the method for isolating nucleic acid of (8), wherein the carrier is a silica particle or a silica derivative particle;

(10): the method for isolating nucleic acid of (8), wherein the carrier is a membrane consisting of silica or a silica derivative;

(11): the method for isolating nucleic acid of (1) to (10), wherein the carrier is a magnetic silica particle or a magnetic silica derivative particle;

(12): the method for isolating nucleic acid of (1) to (11), wherein the washing liquid is a solution containing ethanol; and

(13): the method for isolating nucleic acid of (12), wherein the washing liquid is a solution containing 70% or more ethanol.

According to these inventions of (7) to (13), a contaminant can be efficiently removed, and the inventions are therefore useful. In step 3 (washing step), while use of an aqueous solution containing a high-chaotropic substance is possible, performing the step in a solution containing ethanol is preferable, and a solution containing 70% or more ethanol is particularly preferable. Thereby, the above high-chaotropic substance or the like is suitably dissolved and eliminated.

In a still further aspect, the present invention relates to (14): the method for isolating nucleic acid of (2), wherein a step of washing the carriers and separating them from the washing liquid further comprises at least one of the steps of a) heating the downstream place to dry the carriers retained in the place, and b) blowing air to the downstream place to dry the carriers retained in the place.

According to this invention, since washing liquid can be easily removed by drying from a nucleic acid-adsorbed nucleic acid-binding carrier, residual washing liquid in a nucleic acid solution is eliminated, and the invention is therefore useful.

In a still further aspect, the present invention relates to (15): the method for isolating nucleic acid of (2), wherein the step of eluting nucleic acid from the carrier comprises the steps of: flowing the eluate in the flow path, releasing application of the magnetic field at the downstream place, and eluting nucleic acid from the carrier. According to this invention, elution of nucleic acid from a nucleic acid-adsorbed nucleic acid-binding carrier can be efficiently performed, and the invention is therefore useful.

In a still further aspect, the present invention relates to

(16): the method for isolating nucleic acid of (1) to (15), wherein the eluate comprises an enzyme, an oligonucleotide and a substrate for a nucleic acid amplification reaction;

(17): a method for isolating and amplifying nucleic acid, wherein nucleic acid isolated by the above method for isolating nucleic acid of (1) to (16) is further amplified by a nucleic acid amplification reaction;

(18): the method for isolating and amplifying nucleic acid of (17), wherein the nucleic acid amplification reaction is conducted in a flow path communicating from the flow path for nucleic acid isolation; and

(19): the method for isolating and amplifying nucleic acid of (17) to (18), wherein the nucleic acid amplification reaction is a polymerase chain reaction (PCR).

According to these inventions of (16) to (19), it is possible to provide a nucleic acid amplification reaction that amplifies nucleic acid isolated by the above method for isolating nucleic acid. Examples of a nucleic acid amplification reaction include polymerase chain reaction, ligase chain reaction (LCR: Science, 241:1077-1080, 1988), an RNA-specific amplification method (NASBA method: Nature, 350:91-92, 1991), the SDA method (Proc. Natl. Acad. Sci. USA, 89:392-396, 1992) and the like. However, because of its excellent efficiency in amplification of a trace amount of nucleic acid, and because a reliable result is obtainable is in a short time, polymerase chain reaction, which is widely utilized throughout the world, is particularly preferable. Polymerase chain reaction is a method of nucleic acid amplification normally used in the art, and the protocol therefor is described in, for example, Science, 230:1350-1354, 1985, and a person skilled in the art can conduct a polymerase chain reaction by suitably modifying experiment conditions and the like based on the description of the above literature and the like. Confirmation of an amplification product can be conducted by, for example, subjecting an amplification reactant to electrophoresis under appropriate conditions, treatment with ethidium bromide, and then detection by ultraviolet irradiation. However, a detection method is not particularly limited. According to the present invention, since it is possible to apply nucleic acid solution isolated by the method for isolating nucleic acid directly, in an eluted state, to an amplification reaction, detection at a high sensitivity is enabled.

While the method for isolating nucleic acid of the present invention is not particularly limited, performing the method within an apparatus constituting a cartridge is preferable.

As material of the cartridge used in the method of the present invention, metal, glass, ceramics and the like can be used, but from a viewpoint of processing ease, plastic is desirable. However, in a step of separating nucleic acid, a material that does not adsorb the nucleic acid is preferred. Examples of such material include polyvinyl chloride resin, polyethylene resin, polypropylene resin, polyvinylidene chloride resin, polyurethane resin, nylon resin, polystyrene resin, ABS resin, acrylic resin, fluorocarbon resin, polycarbonate resin, methylpentene resin, phenol resin, melamine resin, epoxy resin and the like. Moreover, as packing to retain hermeticity for a fluid or gas inside a flow path or a valve on a cartridge, an elastomer such as rubber may be used. Examples of this kind of elastomer include rubbers such as natural rubber, butadiene rubber, styrene rubber, isobutylene-isoprene rubber, ethylene propylene rubber, nitrile rubber, acrylic rubber, urethane rubber, silicon rubber and fluorocarbon rubber, and soft polyvinyl chloride resin, polyethylene resin, polypropylene resin, polyvinylidene chloride resin, polyurethane resin, fluorocarbon resin, nylon resin and the like.

Furthermore, in addition to plastic, a component to complete the configuration as a cartridge, a component used in a valve, a component to efficiently transmit heat, and the like, may be a metal such as aluminum, brass, iron, copper, stainless steel, titanium alloy, magnesium alloy and duralumin. In a case of applying heat to a cartridge, the heating part must be of a material capable of maintaining mechanical intensity at a set temperature.

The cartridge is constituted by a flow path for flowing liquid or gas and a reservoir part for storing each solution, a part for conducting reaction, and a part that accumulates nucleic acid-binding magnetic carriers, and is constituted such that solution of the reservoir part is fed into the flow path or reactor part by a gas and/or fluid pump or actuator by means of an apparatus external to the cartridge.

A cartridge used in the present invention has these features, and a series of operations can be completed in an enclosed flow path or in the cartridge containing a flow path. Further, an amplification reaction and a detection reaction can be conducted in continuation in the enclosed flow path or in the cartridge containing the flow path, within the single cartridge.

The present invention further relates to, as a cartridge having a particularly suitable composition,

(20): a cartridge for chemical reaction, having at least one reservoir and/or reaction chamber and at least one flow path, and for applying a chemical reaction to a given ingredient contained in a liquid or gaseous sample or a liquid or gaseous reagent, or a mixed fluid of the sample and the reagent by flowing the sample or the reagent, or the mixed fluid from the at least one reservoir and/or reaction chamber into the at least one flow path, wherein the cartridge feeds a sample solution or a reagent solution or a mixed solution of the sample and the reagent into the flow path using a feeding liquid that is immiscible with and is phase separated from the solution.

Here, a solution to be fed is a liquid of a small amount of a volume of 10 ml or less, preferably 1 ml or less, and more preferably 100 μl or less. In this invention “phase separation” means that, in a case when a solution to be fed is an aqueous solution, an oil-soluble liquid that is immiscible with an aqueous solution is used as a feeding liquid. Any liquid that is immiscible with an aqueous solution can be used as an oil-soluble liquid, for example, edible oil, mineral oil, silicon oil, an organic solvent comprising hydrocarbon (a saturated hydrocarbon solvent, such as hexane, heptane, octane, or the like, or a solvent containing a benzene ring, such as benzene, toluene, xylene, or the like), a solvent containing an oxygen atom (diethyl ether, butanol, ethyl acetate or the like), and a solvent containing a chlorine atom (carbon tetrachloride, chloroform, dichloromethane or the like). According to this invention, in comparison with feeding of a solution using a gas, feeding of the solution can be performed quantitatively. Moreover, according to this invention, when feeding a solution that performs a chemical reaction, by performing to feed using a feeding liquid that is immiscible with the solution, it is possible to prevent the reaction solution being diluted by the feeding liquid. Furthermore, according to this invention, in the case of a solution that performs a chemical reaction, it is possible to prevent the solution from directly contacting an inner wall of a container or flow path, thereby allowing mitigation of adsorption to a inner wall of a substance that involves in a reaction. As a method for feeding a solution, first, the solution to be fed is injected into a flow path into which it is to be fed, and then a feeding liquid may be fed thereto via a pump or the like, and while the method is not particularly limited, preferably, for example, the solution to be fed is embedded in the feeding liquid. A method for embedding is not particularly limited, for example, it can easily be accomplished by previously filling the above flow path of the solution with a first feeding liquid that is immiscible with the solution and is phase separated, to thereby cover the inner walls of the flow path with the first feeding liquid, then introducing a small amount of the solution into the flow path, and subsequently introducing a second feeding liquid. The first feeding liquid and the second feeding liquid are liquid within the range described above, and may be the same or different as long as they are not mutually reactive. To feed a small amount of the solution to be fed in a form in which it is embedded in the feeding liquid, it is required that the surface of a container inner wall have a higher affinity for the feeding liquid than for the solution. For example, in a case when the solution is an aqueous one and the feeding liquid is an oil-soluble one, the surface of an inner wall may be a material having high hydrophobicity.

In a polymerase chain reaction as generally performed, a substance, such as mineral oil, wax or the like, that phase separates with a reaction solution is overlaid in an upper layer of a reaction tube and reaction performed. However, these liquids that phase separate with a reaction solution are added to the upper layer portion for the purpose of preventing evaporation of the reaction solution due to heating of the reaction solution, and are not for the purpose of feeding or adsorption prevention in a reaction system inside a flow path. In practice, as a method for performing polymerase chain reaction without adding the oil, a method is also widely used in which, after making a tube a closed system, the temperature of the entire tube is changed. Accordingly, it is clearly distinguished from the present invention.

The present invention further relates to (21): a cartridge for chemical reaction, having at least one reservoir and/or reaction chamber and at least one flow path, and for applying a chemical reaction to a given ingredient contained in a liquid or gaseous sample or a liquid or gaseous reagent, or a mixed fluid of the sample and the reagent by flowing the sample or the reagent, or the mixed fluid from the at least one reservoir and/or reaction chamber into the at least one flow path, wherein the cartridge has the following features:

1. having a multilayered structure of three or more layers in which at least one of a tabular member for hermeticity comprising an elastomer and at least two of a tabular member for a base plate comprising material having a lower elasticity and a higher degree of hardness than the elastomer are alternately interposed and crimped;

2. the flow path comprises at least one member selected from the group consisting of: a groove and/or a hole provided in the tabular member for a base plate, a groove and/or a hole provided in the tabular member for hermeticity, and an aperture formed by transformation of a part of the tabular member for hermeticity due to pressure of the sample, the reagent or the mixed fluid; and

3. the reservoir and/or reaction chamber comprises a groove and/or a hole provided in the tabular member for hermeticity and/or the tabular member for a base plate;

and (22): a cartridge for chemical reaction, having at least one reservoir and/or reaction chamber and at least one flow path, and for applying a chemical reaction to a given ingredient contained in a liquid or gaseous sample or a liquid or gaseous reagent, or a mixed fluid of the sample and the reagent by flowing the sample or the reagent, or the mixed fluid from the at least one reservoir and/or reaction chamber into the at least one flow path;

wherein the cartridge feeds a sample solution or a reagent solution or a mixed solution of the sample and the reagent into the flow path using a feeding liquid that is immiscible with and is phase separated from the solution, and has the following features:

1. having a multilayered structure of three or more layers in which at least one sheet of a tabular member for hermeticity comprising an elastomer and at least two sheets of a tabular member for a base plate comprising material having a lower elasticity and a higher degree of hardness than the elastomer are alternately interposed and crimped;

2. the flow path comprises at least one member selected from the group consisting of: a groove and/or a hole provided in the tabular member for a base plate, a groove and/or a hole provided in the tabular member for hermeticity, and an aperture formed by transformation of a part of the tabular member for hermeticity due to pressure of the sample, the reagent or the mixed fluid; and

3. the reservoir and/or reaction chamber comprises a groove and/or a hole provided in the tabular member for hermeticity and/or the tabular member for a base plate.

Examples of a material having elasticity used in a tabular member for hermeticity (hereinafter abbreviated to “sealing plate”) include rubbers such as natural rubber, butadiene rubber, styrene rubber, isobutylene-isoprene rubber, ethylene propylene rubber, nitrile rubber, acrylic rubber, urethane rubber, silicon rubber and fluorocarbon rubber, and soft polyvinyl chloride resin, polyethylene resin, polypropylene resin, polyvinylidene chloride resin, polyurethane resin, fluorocarbon resin, nylon resin and the like.

Further, examples of a material that can be used in a tabular member for a base plate (hereinafter referred to as “base plate”) include plastics such as unplasticized polyvinyl chloride (UPVC), polystyrene resin, ABS resin, polyethylene resin, polypropylene resin, nylon resin, acrylic resin, fluorocarbon resin, polycarbonate resin, methylpentene resin, polyurethane resin, phenol resin, melamine resin and epoxy resin; metals such as aluminum, brass, iron, copper, stainless steel, titanium alloy, magnesium alloy and duralumin; glass, ceramics, and the like.

In the present invention, the combination that a sealing plate has a higher modulus of elasticity than a base plate, and conversely, a base plate has a higher degree of hardness than a sealing plate, is important. For example, a combination may be silicon rubber for a sealing plate and fluorocarbon resin for a base plate, or may be fluorocarbon resin for a sealing plate and stainless steel for a base plate. Moreover, it is essential that, when conducting a target chemical reaction, both a sealing plate and a base plate be of a material that does not effect the chemical reaction.: Further, in the case of performing a reaction in a liquid, it is necessary they be of a material that is substantially impervious to liquid, and in the case of performing a gas reaction, it is necessary they be of a material that is substantially impervious to gas.

A flow path inside the cartridge may be constructed by processing of a sealing plate or may be constructed by processing of a base plate. Regarding the size of a flow path, after crimping the sealing plate and the base plates, it is necessary that a width and depth formed by transformation of the sealing plate and the base plates by such pressure are such that a flow path does not become blocked. The width and depth of a constructed flow path will vary depending on the elasticity modulus of a material used in a sealing plate or base plate and the crimping pressure employed in forming the sealing plate and the base plates into a multilayered structure. In substance, in order to fulfill a function as a flow path of a cartridge, a groove of a size having a depth of 10 micron and a width of 10 micron, or greater, is required. An excessively large flow path for completing one series of reactions inside one cartridge will result in loss of the advantage of the POCT. The upper size limit of a practicable flow path is about a width of 5 mm and a depth of 1 cm. As a reservoir part for storing a reaction solution or the like, a groove or hole having a greater depth and width than this can be used. The upper size limit of a reservoir as a storage space for a reaction solution is about a width of 10 cm and a depth of 10 cm. In the present invention, in order to construct a multilayered structure comprising a base plate and a sealing plate, it is necessary that a method for crimping does not interfere with a multilayered structure forming a flow path, reservoir, reaction chamber and the like.

Accordingly, examples of the method include, but are not limited to, a method for crimping, at a part not interfering with the multilayered structure forming a flow path, reservoir, reaction chamber and the like, by piercing a penetrating hole and using a screw and a nut, or by piercing a penetrating hole and using a rivet, or by piercing a non-penetrating hole and using a screw, or by using a spring from outside a multilayered structure, or by adhesion of an external part of a multilayered structure. According to this invention, using a base plate or sealing plate having a groove or hole processed therein, it is possible to construct a cartridge that performs a chemical reaction without using advanced techniques such as gluing together. Furthermore, since the cartridge does not require irreversible processing, such as adhesion or the like, recycling of a processed cartridge by disassembly and washing is also enabled. Here, a cartridge is constructed such that a solution of a reservoir part or the like is fed to a flow path or reactor part by an actuator or pump of gas and/or liquid provided outside the cartridge.

In a still further aspect, the present invention relates to

(23): the cartridge for chemical reaction of (21) and (22), wherein the cartridge comprises at least one valve that controls opening and closing of the flow path;

(24): the cartridge for chemical reaction of (23), wherein at least one of the valves is a valve controlling opening and closing of a flow path on the cartridge, having a rod-shaped element, movement of the rod-shaped element being possible with respect to a flow path on the cartridge, the rod-shaped element having an open part and a closed part, the open part being of a structure such that a projected area to the vertical plane with respect to a movement direction is smaller than that of the closed part, and by movement of the rod-shaped element a flow path on the cartridge and an open part of the rod-shaped element communicate, thus opening the valve, and by movement of the rod-shaped element a flow path on the cartridge is blocked by a closed part of the rod-shaped element, thus closing the valve;

(25): the cartridge for chemical reaction of (23), wherein at least one of the valves is a valve controlling opening and closing of the flow path by controlling formation of an aperture formed by transformation of a part of a tabular member for hermeticity caused by pressure of the sample, the reagent and/or the mixed fluid; and

(26): the cartridge for chemical reaction of (23) to (25), wherein the cartridge comprises a control apparatus controlling opening and closing of at least one of the valves by an actuator.

In the above, “the open part being of a structure such that a projected area to the vertical plane with respect to a movement direction is smaller than that of the closed part” means, for example, that the rod-shaped element is subjected to additional working such as insertion of a notch, a groove, a hole, or the like, and these constitute an open part of the rod-shaped element.

The above valve is not particularly limited and, for example, in a flow path in which, by pressure of the sample, the reagent or the mixed fluid, a part of a sealing plate is formed by transformation in the direction of a groove and/or hole different to the flow path provided in one base plate crimped to the sealing plate, wherein the flow path is constituted by an aperture between the sealing plate and another base plate of a side opposite to the base plate, the valve may be a means functioning as a valve controlling opening and closing of the flow path by exerting pressure on a part of the sealing plate from the side of the groove and/or hole different to the flow path to suppress formation of the aperture to thereby block the flow path, or reducing the pressure to release the suppression and thereby open the flow path. According to the inventions of (23) to (26), adoption in a cartridge for chemical reaction of a valve of a simple and convenient constitution is enabled. Therefore, when conducting a chemical reaction, in the handling of a liquid or gas, a valve enables the prevention of mixing of substances for which reaction is to be avoided or the prevention of reflux of a liquid or gas, thus allowing easy control of a chemical reaction on the cartridge.

In a still further aspect, the present invention relates to

(27): the above cartridge for chemical reaction, wherein the temperature in the cartridge is controlled by heating or cooling at least one part of the cartridge;

(28): the above cartridge for chemical reaction, wherein the temperature is controlled by heating and/or cooling at least two places to respectively different temperatures; and

(29): the cartridge for chemical reaction of (28), wherein at least two places of the flow path are heated and/or cooled to control at respectively different temperatures, and the sample, the reagent or the mixed fluid is fed back and forth inside the flow path to apply a chemical reaction to a given ingredient in the sample, the reagent, or the mixed fluid.

According to the invention of (27) to (29), a chemical reaction requiring heating or cooling can be performed on a cartridge for chemical reaction, thus broadening the range of chemical reactions that can be adapted to the cartridge. In the case of performing heating, a heating part of a cartridge must be of a material that can maintain mechanical intensity and also maintain a flow path of a liquid and/or gas at a set temperature. In order to maintain mechanical intensity and transmit heat efficiently, in addition to a plastic, it is also possible to use a metal such as aluminum, brass, iron, copper, stainless steel, titanium alloy, magnesium alloy, duralumin or the like as material of the cartridge. A means for controlling temperature is not particularly limited, and, for example, a water bath at a set temperature can be performed or various types of heating devices or cooling devices can be used.

In a further aspect, the present invention relates to (30): the cartridge for chemical reaction of (27) to (29), wherein the chemical reaction is a nucleic acid amplification reaction; and (31) the cartridge for chemical reaction of (30), wherein the nucleic acid amplification reaction is a polymerase chain reaction (PCR).

According to the inventions of (30) to (31), it is possible to conduct a nucleic acid amplification reaction on a cartridge for chemical reaction. Examples of a nucleic acid amplification reaction include a polymerase chain reaction, a ligase chain reaction (LCR: Science, 241:1077-1080, 1988), an RNA-specific amplification method (NASBA method: Nature, 350:91-92, 1991), the SDA method (Proc. Natl. Acad. Sci. USA, 89:392-396, 1992), and the like. However, because of its excellent efficiency in amplification of a trace amount of nucleic acid, and because a reliable result is obtainable is in a short time, polymerase chain reaction, which is widely utilized throughout the world, is particularly preferable. Polymerase chain reaction is a method of nucleic acid amplification normally used in the art, and the protocol therefor is described in, for example, Science, 230:1350-1354, 1985, and a person skilled in the art can conduct a polymerase chain reaction by suitably modifying experiment conditions and the like based on the description of the above literature and the like. Confirmation of an amplification product can be conducted by, for example, subjecting an amplification reactant to electrophoresis under appropriate conditions, and, for example, treatment with ethidium bromide and then detection by ultraviolet irradiation. However, a detection method is not particularly limited.

In a still further aspect, the present invention relates to

(32): a cartridge for nucleic acid isolation, comprising at least one of:

a nucleic acid-binding carrier;

a solution for adsorbing/releasing nucleic acid that releases nucleic acid from a material containing nucleic acid and adsorbs it on the nucleic acid-binding carrier;

a washing liquid that washes a nucleic acid-binding carrier on which nucleic acid is adsorbed; and

an eluate that elutes nucleic acid from a nucleic acid-binding carrier on which nucleic acid is adsorbed;

(33): a cartridge for nucleic acid isolation having the structure of the above cartridge for chemical reaction, which comprises at least one of:

a nucleic acid-binding carrier;

a solution for adsorbing/releasing nucleic acid that releases nucleic acid from a material containing nucleic acid and adsorbs it on the nucleic acid-binding carrier;

a washing liquid that washes a nucleic acid-binding carrier on which nucleic acid is adsorbed; and

an eluate that elutes nucleic acid from a nucleic acid-binding carrier on which nucleic acid is adsorbed; and

(34): the cartridge for nucleic acid isolation of (33), wherein the chemical reaction comprises a nucleic acid isolation reaction comprising the steps of:

1. mixing a material containing nucleic acid and a solution for adsorbing/releasing nucleic acid, and contacting a mixed solution thereof with nucleic acid-binding carriers to prepare nucleic acid-adsorbed nucleic acid-binding carriers;

2. isolating the nucleic acid-adsorbed nucleic acid-binding carriers;

3. washing the nucleic acid-adsorbed nucleic acid-binding carriers;

4. drying the nucleic acid-adsorbed nucleic acid-binding carriers; and

5. eluting nucleic acid from the nucleic acid-binding carriers.

According to the inventions of (32) to (34), it is possible to construct a cartridge for nucleic acid isolation that easily accomplishes isolation of nucleic acid from a sample containing nucleic acid on a cartridge. Moreover, according to the invention of (34), after washing nucleic acid-adsorbed nucleic acid-binding carriers with washing liquid, by blowing in gas (air or nitrogen) in a heated state, it is possible to dry the carriers and remove washing liquid without opening a cover. In this case, it is important that a gas that is blown in is not contaminated with other microorganisms or nucleic acids. To ensure this, a gas that is blown in can be passed through a filter before use, and a step for isolating microorganisms can also be included. Preferably, a material used in the cartridge for nucleic acid isolation is a material which does not adsorb the nucleic acid.

In a further aspect, the present invention relates to

(35): the cartridge for nucleic acid isolation of (32), wherein step 1 is conducted under heating;

(36): the cartridge for nucleic acid isolation of (35), wherein heating of step 1 is conducted at a temperature of 60° C. or higher and 130° C. or lower;

(37): the cartridge for nucleic acid isolation of (35) to (36), wherein heating of step 1 is conducted at a temperature of 80° C. or higher and 1100° C. or lower;

(38): the cartridge for nucleic acid isolation of (35) to (37), wherein heating of step 1 is conducted at a temperature of 90° C. or higher and 100° C. or lower; and

(39): the cartridge for nucleic acid isolation of (35) to (38), wherein heating of step 1 is conducted for 1 minute or more and 1 hour or less.

According to the inventions of (35) to (39), it is possible to construct a cartridge for nucleic acid isolation wherein a factor imparting an inhibiting effect to an amplification reaction does not remain in an isolated nucleic acid solution.

In a still further aspect, the present invention relates to

(40): the cartridge for nucleic acid isolation of (32) to (39), wherein the solution for adsorbing/releasing nucleic acid is a solution containing a high-chaotropic substance;

(41): the cartridge for nucleic acid isolation of (32) to (40), wherein the carrier is silica or a silica derivative;

(42): the cartridge for nucleic acid isolation of (41), wherein the carrier is a silica particle or a silica derivative particle;

(43): the cartridge for nucleic acid isolation of (41), wherein the carrier is a membrane consisting of silica or a silica derivative;

(44): the cartridge for nucleic acid isolation of (32) to (43), wherein the washing liquid is a solution containing ethanol; and

(45): the cartridge for nucleic acid isolation of (44), wherein the washing liquid is a solution containing 70% or more ethanol.

According to the inventions of (40) to (45) a cartridge for nucleic acid isolation that efficiently excludes a contaminant can be constructed, and the cartridge is therefore useful.

In a further aspect, the present invention relates to

(46): the cartridge for nucleic acid isolation of (32) to (45), wherein the carrier is a magnetic silica particle or a magnetic silica derivative particle;

(47): a cartridge for nucleic acid isolation, which is a cartridge for isolating nucleic acid from a nucleic acid-adsorbed nucleic acid-binding magnetic carrier, wherein the cartridge comprises a flow path for nucleic acid isolation, and wherein a magnetic field capable of retaining the carrier can be applied in at least two places along the flow path; and

(48): a cartridge for nucleic acid isolation, comprising:

a reaction chamber for mixing and reacting a material containing nucleic acid, a nucleic acid-binding magnetic carrier and a solution for adsorbing/releasing nucleic acid;

a flow path for nucleic acid isolation, wherein in at least two places along the flow path a magnetic field capable of retaining the carrier can be applied;

a reservoir for storing the solution for adsorbing/releasing nucleic acid;

a flow path linking the reservoir for storing the solution for adsorbing/releasing nucleic acid and the reaction chamber;

a flow path linking the reaction chamber and the flow path for nucleic acid isolation;

at least one reservoir for storing a solution for washing;

at least one flow path for washing which links the reservoir for storing a solution for washing and at least one member selected from the group consisting of: the reaction chamber, the flow path linking the reservoir for storing a solution for adsorbing/releasing nucleic acid and the reaction chamber, the flow path linking the reaction chamber and the flow path for nucleic acid isolation, and the flow path for nucleic acid isolation;

a reservoir for storing a solution for eluting nucleic acid; and

a flow path linking the reservoir for storing a solution for eluting nucleic acid and at least one member selected from the group consisting of: the reaction chamber, the flow path linking the reservoir for storing a solution for adsorbing/releasing nucleic acid and the reaction chamber, the flow path linking the reaction chamber and the flow path for nucleic acid isolation, the flow path for nucleic acid isolation, and the flow path for washing.

According to the inventions of (46) to (48), when washing the carriers on a cartridge, since the carriers are dispersed in washing liquid, it is possible for washing liquid to efficiently wash the carriers and to efficiently eliminate any contaminants other than nucleic acid from nucleic acid-binding carriers. Further, according to these inventions, when washing the carriers on a cartridge, since an aggregate of the carriers is dispersed at the time of washing, the possibility of causing a blockage is low. Further, according to these inventions, it is possible to accomplish a series of operations for extracting and isolating the nucleic acid from a material containing nucleic acid using a nucleic acid-binding magnetic carrier, inside a cartridge for nucleic acid isolation.

In a still further aspect, the present invention relates to (49): the cartridge for nucleic acid isolation of (32) to (48), wherein a chemical reaction further comprises a nucleic acid amplification reaction that amplifies a nucleic acid isolated by a nucleic acid isolation reaction; and (50): the cartridge for nucleic acid isolation of (49), wherein a nucleic acid amplification reaction is a polymerase chain reaction (PCR). According to the inventions of (49) to (50), after nucleic acid is isolated, it is possible to subsequently perform a nucleic acid amplification reaction on a cartridge for nucleic acid isolation, thus enabling rapid detection of a nucleic acid.

In the present invention, the term “nucleic acid” refers to DNA (deoxyribonucleic acid) or RNA (ribonucleic acid), and to DNA or RNA included in the target material.

In the present invention, examples of a material containing nucleic acid include sputum, saliva, urine, stool, semen, blood, tissue, organ, or another body fluid, the foregoing being clinical specimens used in diagnosis of a human patient, or a fraction of these body fluids, food used in a test for microorganism contamination, drinking water, soil, effluent, river water, seawater, wiping solution, a cotton wipe and the like. Further, a bacterial suspension of bacteria such as Escherichia coli, a culture medium, or a fungus body (colony) cultured on a solid medium can be used.

A nucleic acid-binding magnetic carrier, a solution for adsorbing/releasing nucleic acid, a washing liquid and an eluate used in the present invention can be used in combination. More specifically, a combination can be used in which nucleic acid released in a liquid layer in a solution for adsorbing/releasing nucleic acid binds to a nucleic acid-binding magnetic carrier, the binding is maintained in a washing liquid, and the adsorbed nucleic acid is eluted in an eluate. For example, a combination can be used in which a nucleic acid-binding magnetic carrier is a substance containing silica or its derivative, a solution for adsorbing/releasing nucleic acid is a solution of a high-chaotropic substance, a washing liquid is an alcohol solution, and an eluate is water or an aqueous solution of a salt concentration of 1 M or less of pH 6 to 9. Examples of a high-chaotropic substance include guanidine, thiocyanate ion, iodine ion, urea and the like. Further, a solution for adsorbing/releasing nucleic acid may be one in which a releasing solution and an adsorbing solution are different solutions. As a releasing solution, for example, a protease such as pronase, a sugar chain-degrading enzyme such as lysozyme, a lipid-degrading enzyme such as lipase, a surfactant, urea, a high-chaotropic substance such as guanidine or thiosulfate, and a metal ion chelating agent such as EDTA or the like can be used alone or in combination.

Any carrier that is normally used in the art can be suitably used as a nucleic acid-binding carrier used in the cartridge for chemical reaction of the present invention, and in the present invention the quantity of a carrier can be freely modified according to the quantity of nucleic acid of a specimen. Moreover, as elution is possible using a small quantity of eluate, in particular, a silica particle or a silica derivative particle, or a magnetic silica particle or a magnetic silica derivative particle, are preferable.

Further, as a nucleic acid-binding carrier, a membrane consisting of silica or a silica derivative may be used.

In the present invention, a silica particle is a high molecular weight polymer in which Si (silicon) and O (oxygen) are bound. For example, silica gel, silica glass, silicon oxide, silicate and the like. Further, examples of a silica derivative include a substance in which an organic compound is chemically bound to the silica. The production method thereof is not particularly limited. Accordingly, those that are generally available on the market can be used. Examples of silica particles used in the present invention include SiO₂ manufactured by Sigma, amorphous silicon oxide, glass powder and the like; and examples of a silica derivative particle include alkylsilica, aluminium silicate, active silica having —NH₂, a latex particle and the like.

In the present invention, while a magnetic substance used as a nucleic acid-binding magnetic carrier is not particularly limited as long as it is a substance having magnetism, a substance is used which generates strong magnetism and binds together upon application of a magnetic field, and which, upon termination of the magnetic field, loses magnetism and disperses. Examples of a substance exhibiting such properties include an alloy or the like having spinel ferrite or plumbite ferrite, iron, nickel, cobalt or the like as a principal component. Further, examples of a magnetic silica particle or magnetic silica derivative particle used in the present invention include a particle included in MagExtractor™ Kit manufactured by Toyobo Co., Ltd. (hereinafter referred to as MagExtractor™m), a particle included in MagNA Pure LC DNA Isolation Kit manufactured by Roche, and the like. When using a magnetic silica particle or magnetic silica derivative particle as a nucleic acid-binding carrier, a carrier can be simply collected by utilizing a magnet.

In the present invention, a magnetic field which accumulates nucleic acid-binding magnetic carriers in an accumulation place of a flow path or a cartridge is supplied from an external device which operates a flow path or cartridge. An external device is equipped with a part that generates a magnetic field by means of an electromagnet or a permanent magnet, which is provided such that, in accordance with a step of isolating nucleic acid performed inside a cartridge, a magnetic field is supplied to an accumulation position of the magnetic carriers, and when dispersing the magnetic carriers in a flow path, the magnetic field is extinguished.

This specification includes part or all of the contents as disclosed in the specification and/or drawings of Japanese Patent Application Nos. 2001-202502, 2001-313511, 2001--393445 and 2002-189729 which are priority documents of the present invention.

### BRIEF DESCRIPTION OF THE DRAWINGS

FIG. 1-a shows the result of agarose gel electrophoresis of Example 1. The left margin shows the size of the DNA molecular weight marker (unit: base pair).

Lane 1: DNA molecular weight marker

Lane 2: PCR amplification product of extracted nucleic acid at 98° C.

Lane 3: PCR amplification product of extracted nucleic acid at 90° C.

Lane 4: PCR amplification product of extracted nucleic acid at 80° C.

Lane 5: PCR amplification product of extracted nucleic acid at 70° C.

Lane 6: PCR amplification product of extracted nucleic acid at 60° C.

Lane 7: PCR amplification product of extracted nucleic acid at 50° C.

Lane 8: PCR amplification product of extracted nucleic acid at 40° C.

Lane 9: PCR amplification product of extracted nucleic acid at 30° C. Lane 10: negative control

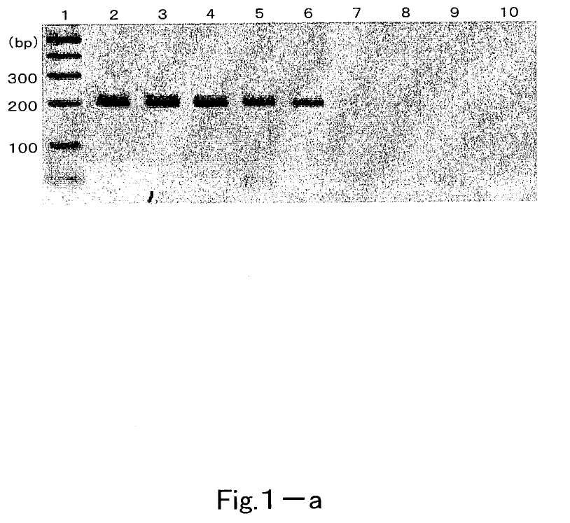

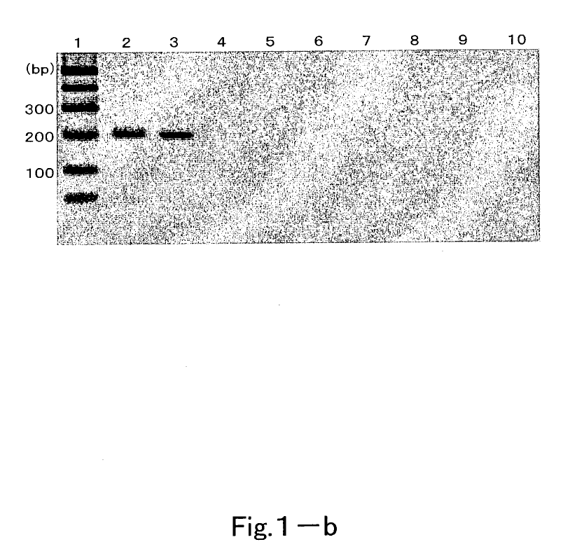

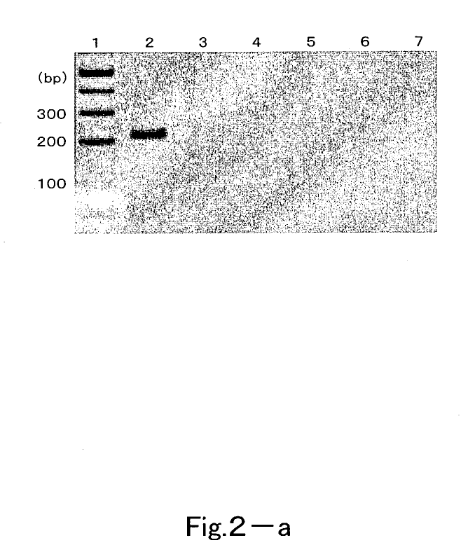

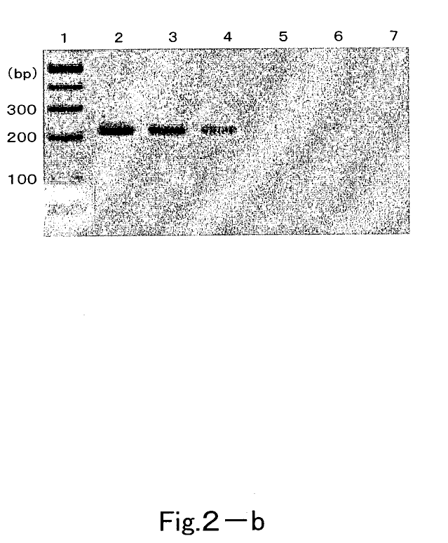

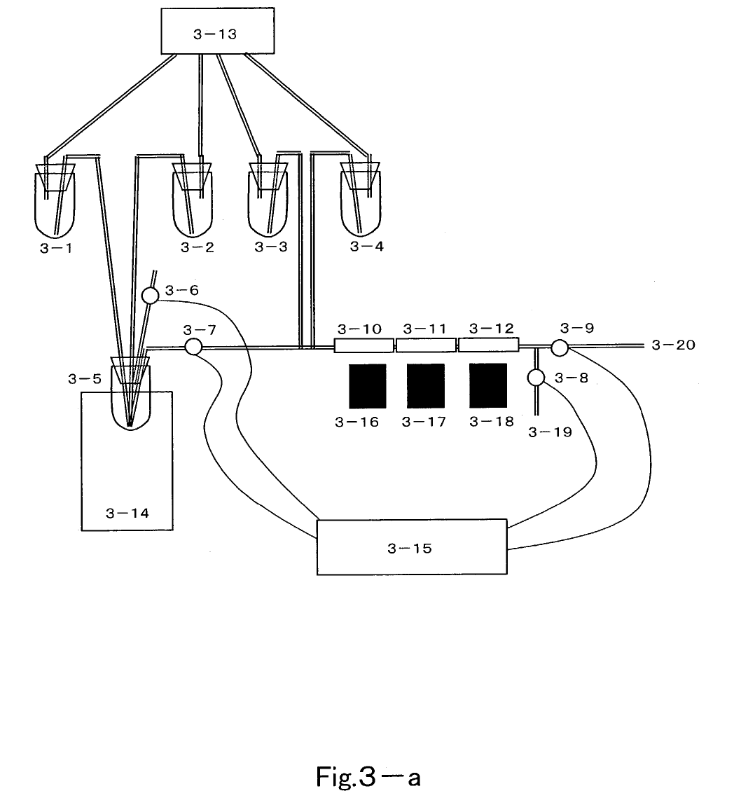

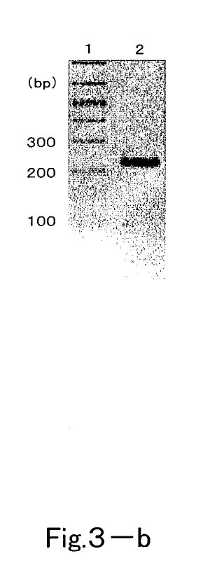

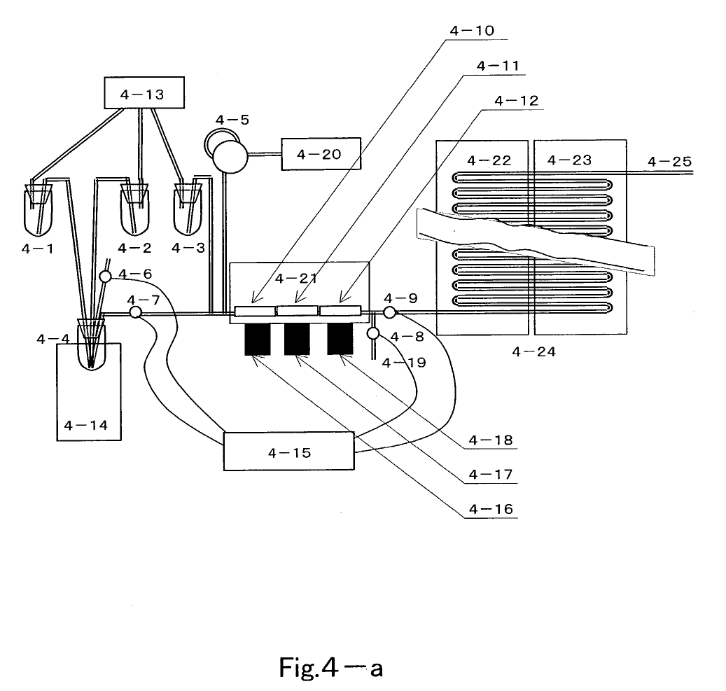

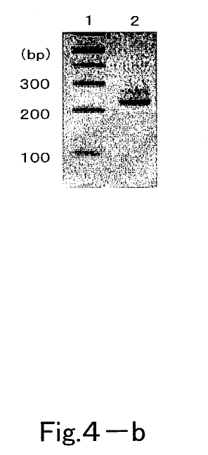

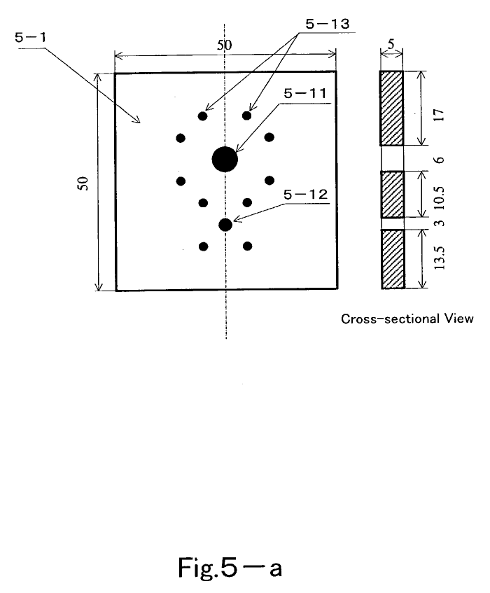

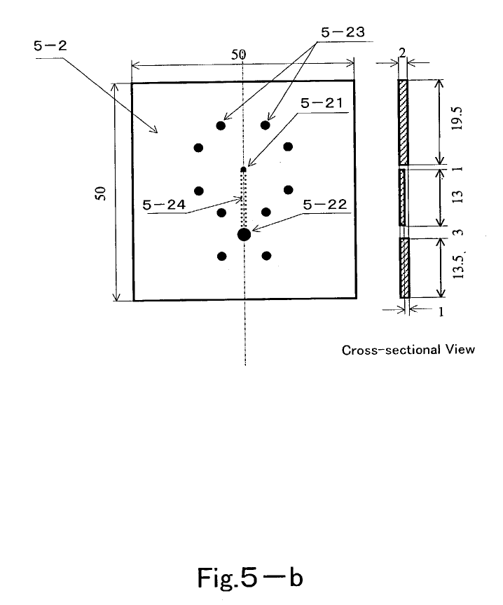

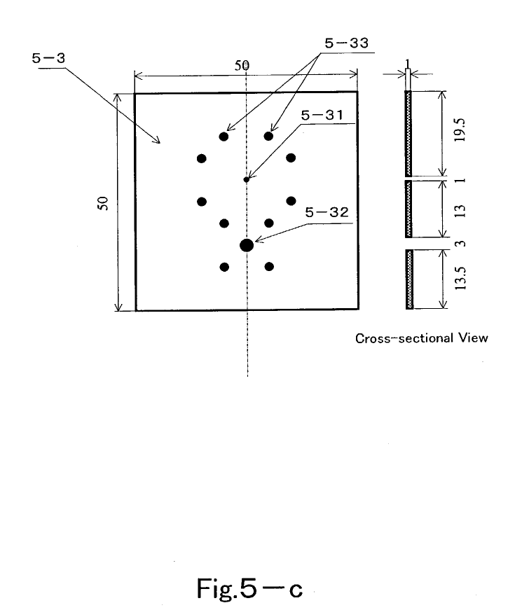

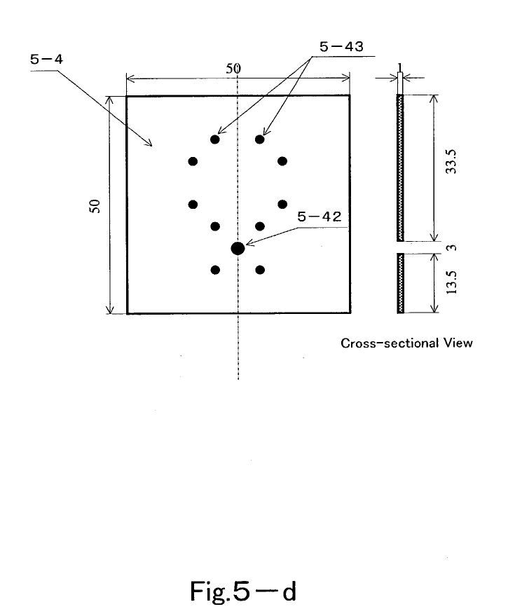

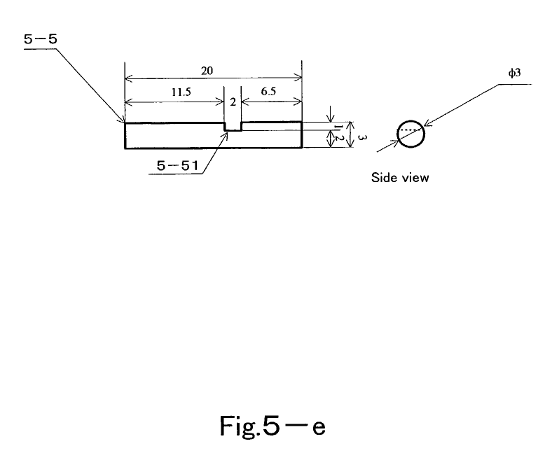

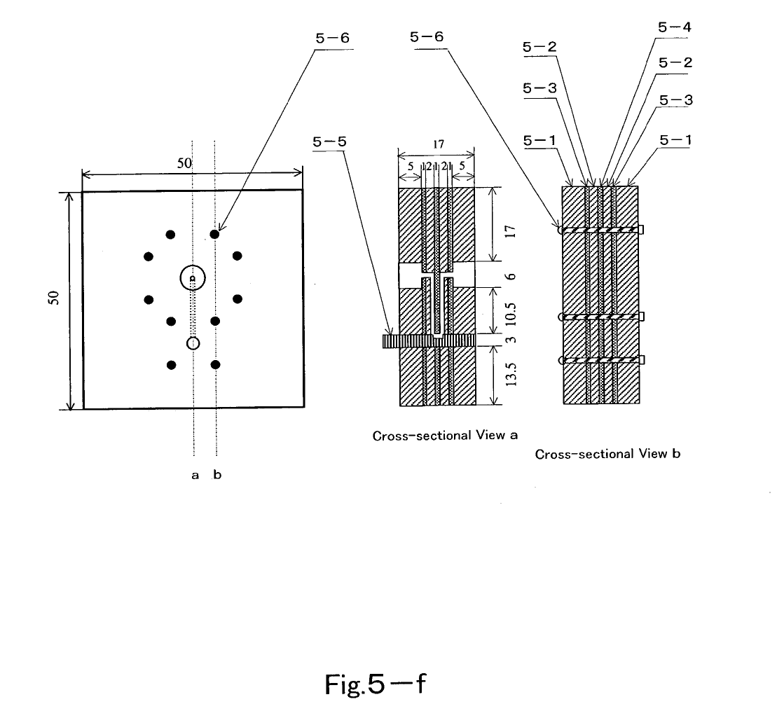

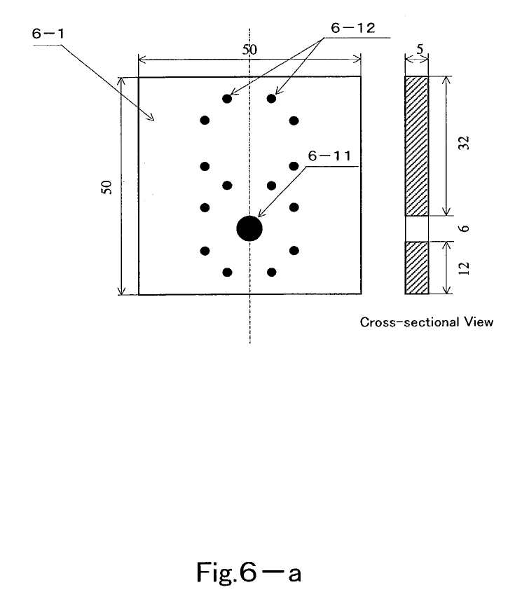

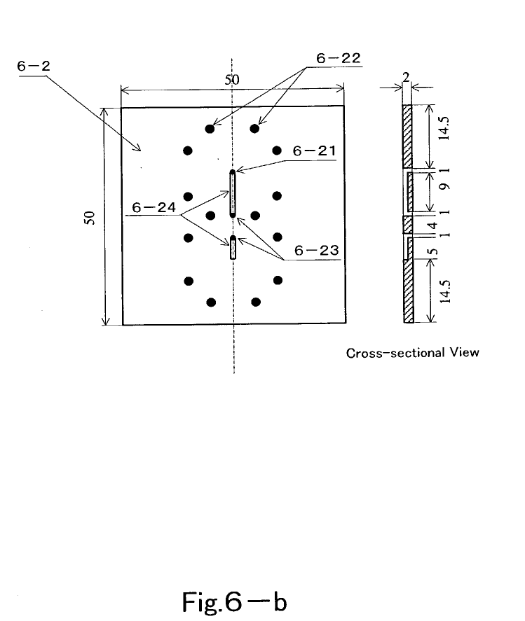

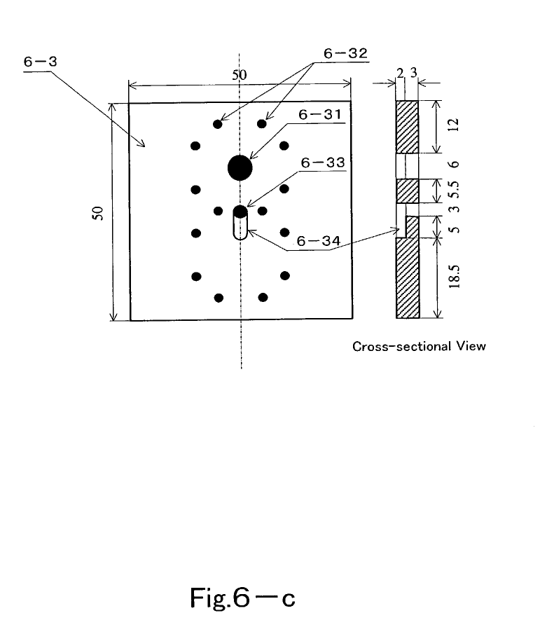

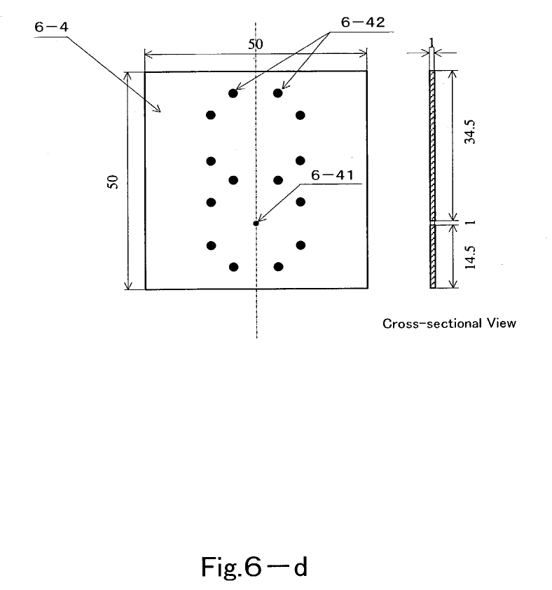

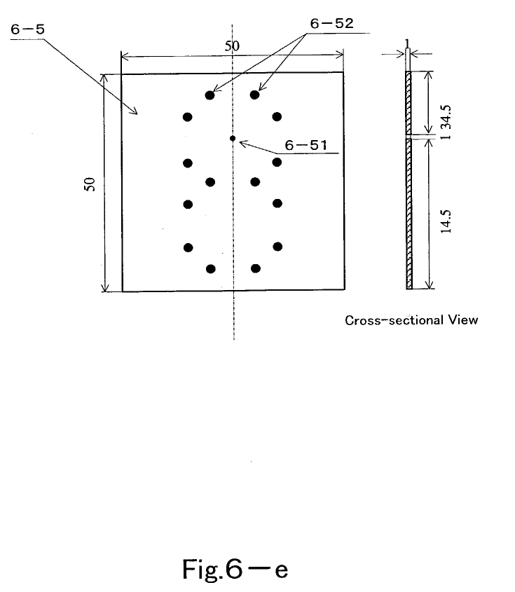

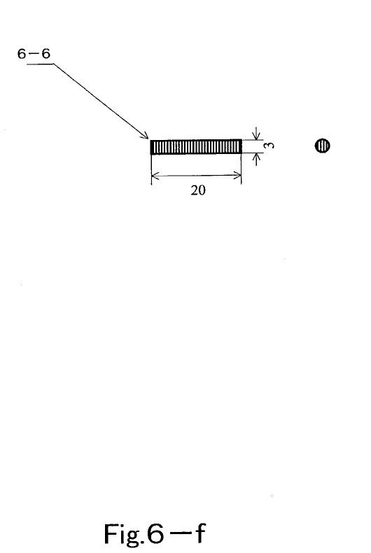

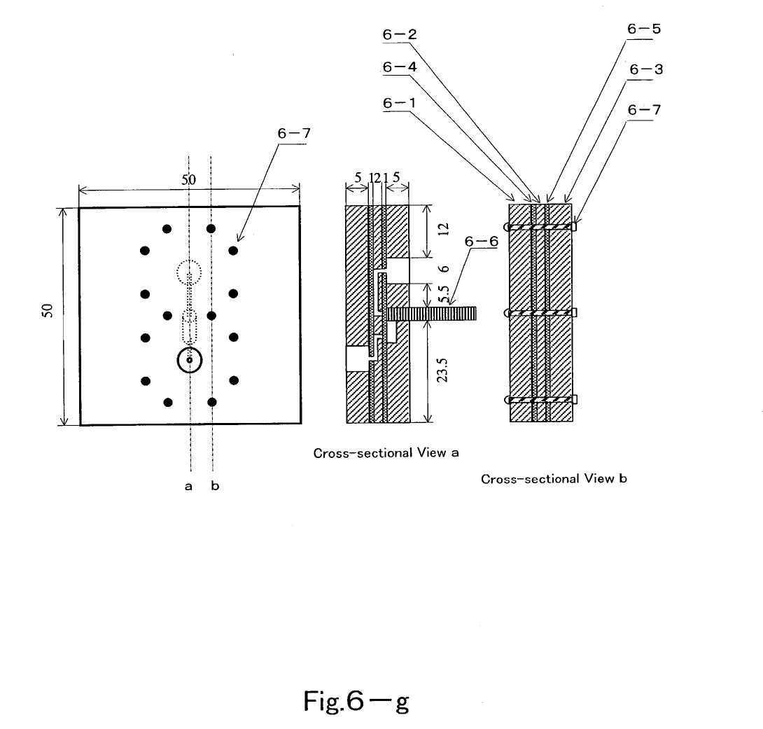

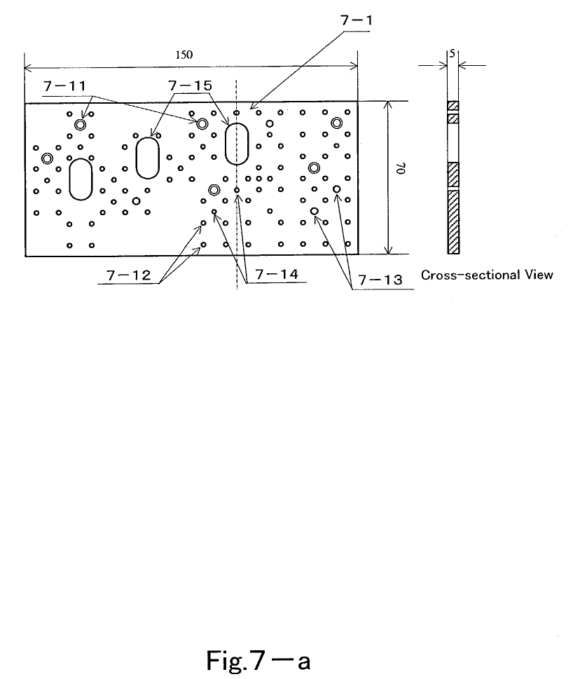

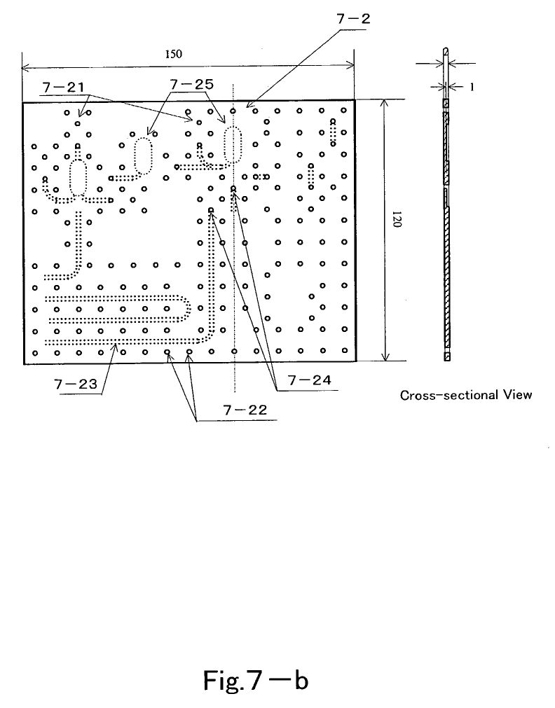

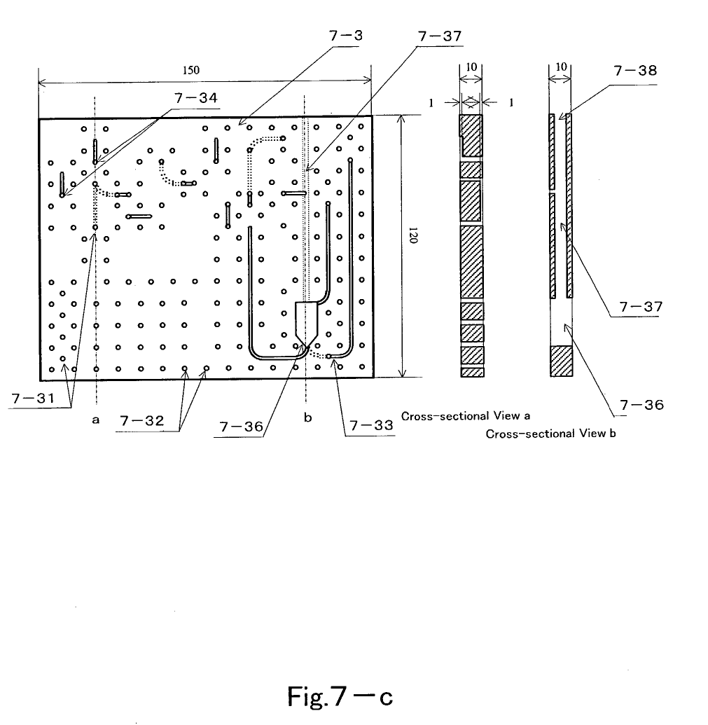

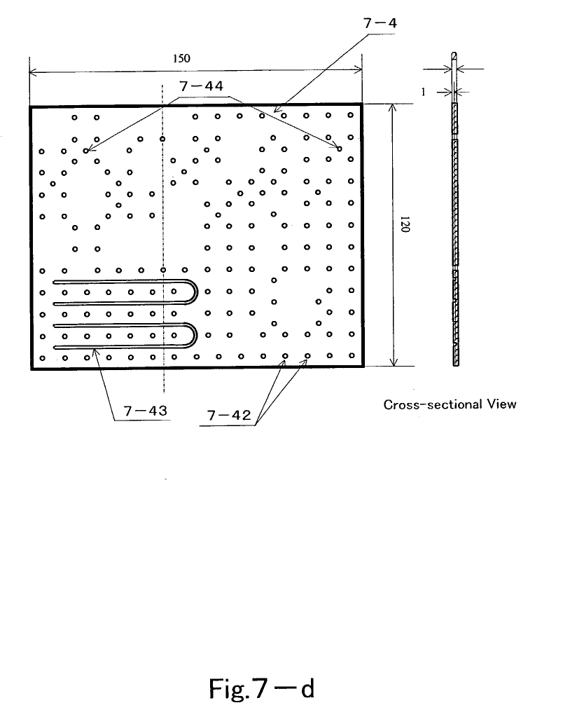

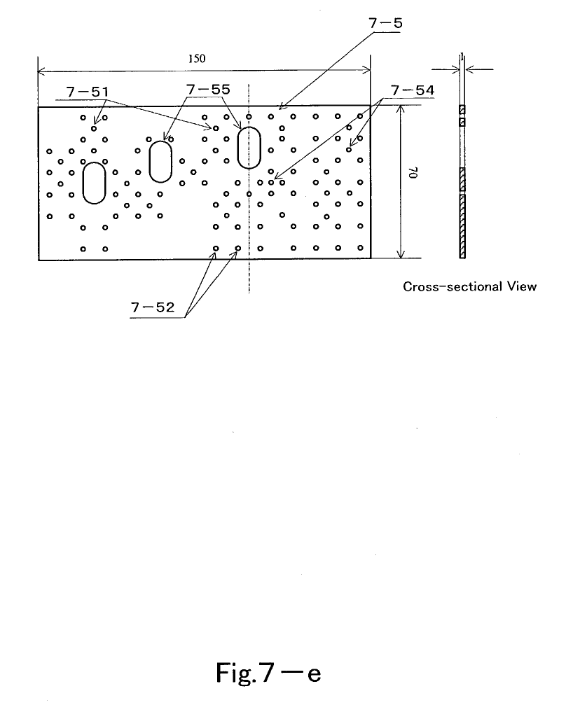

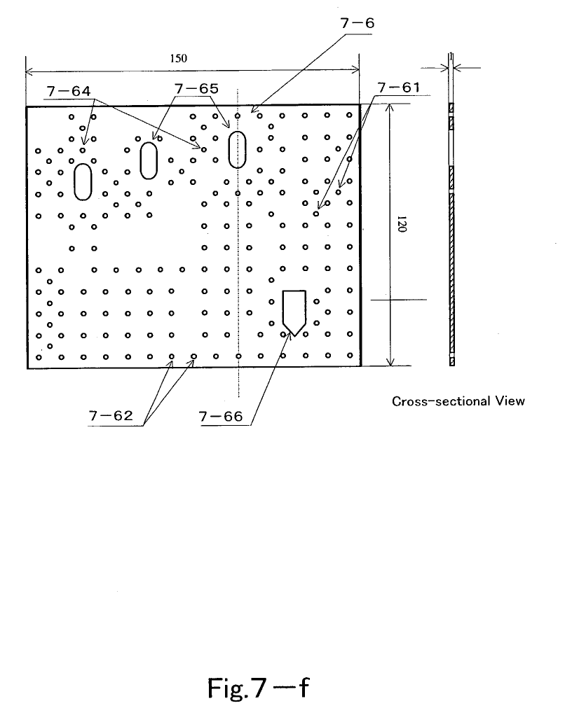

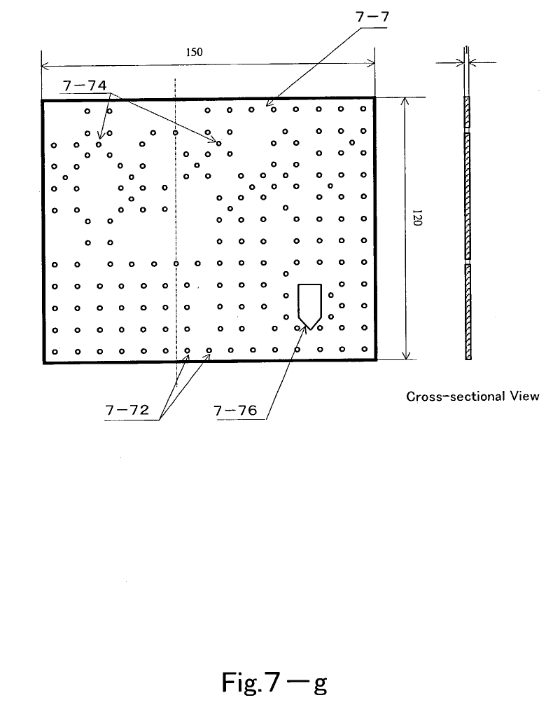

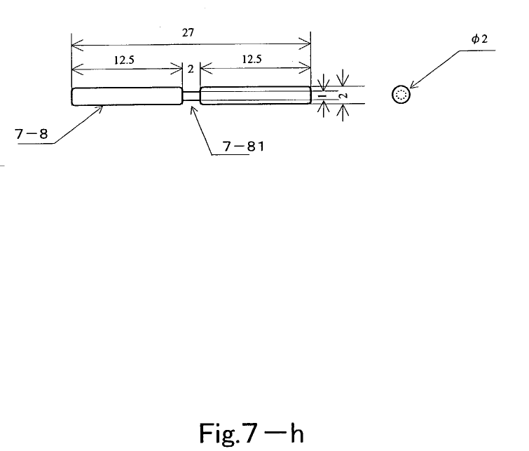

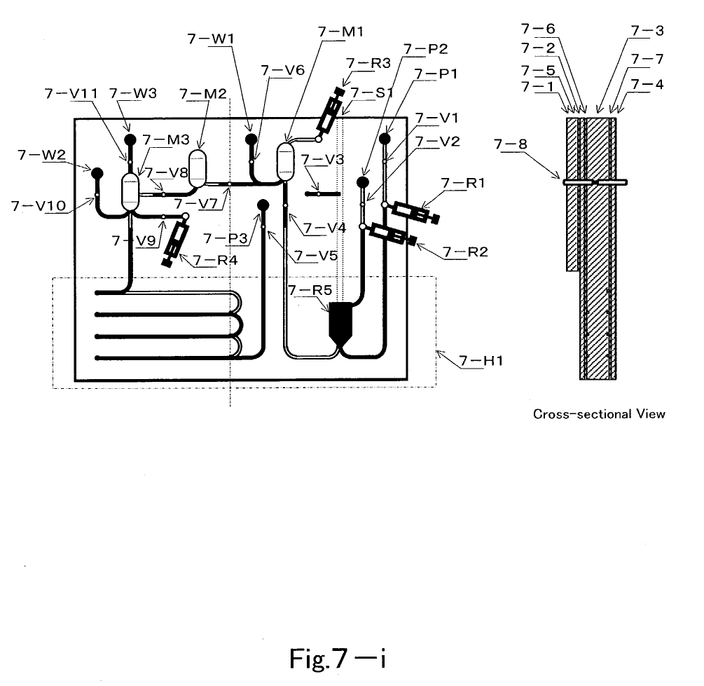

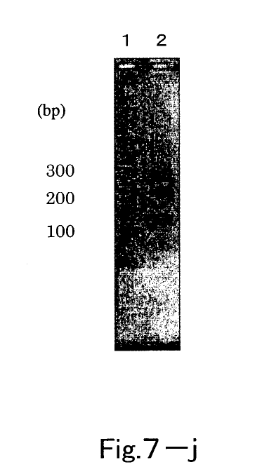

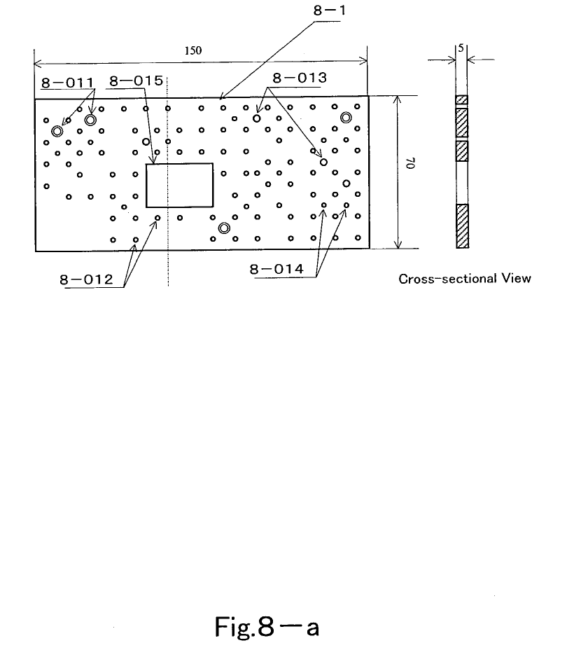

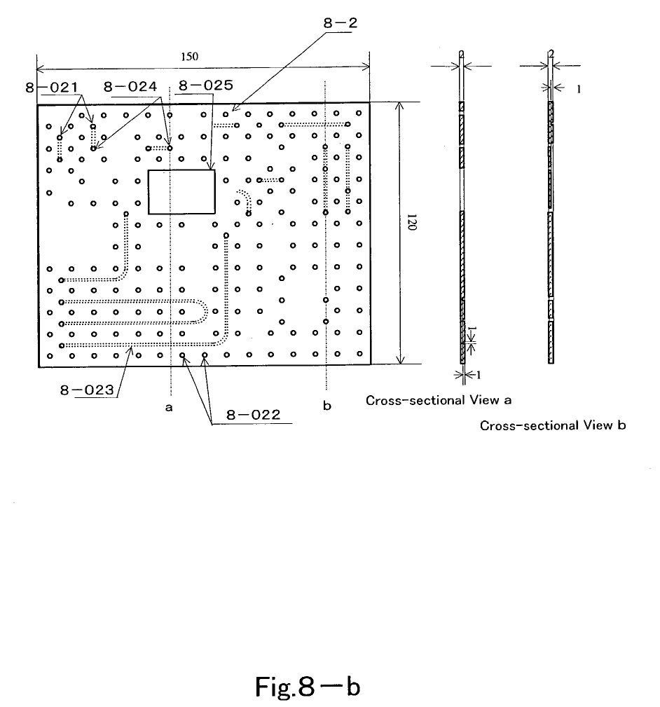

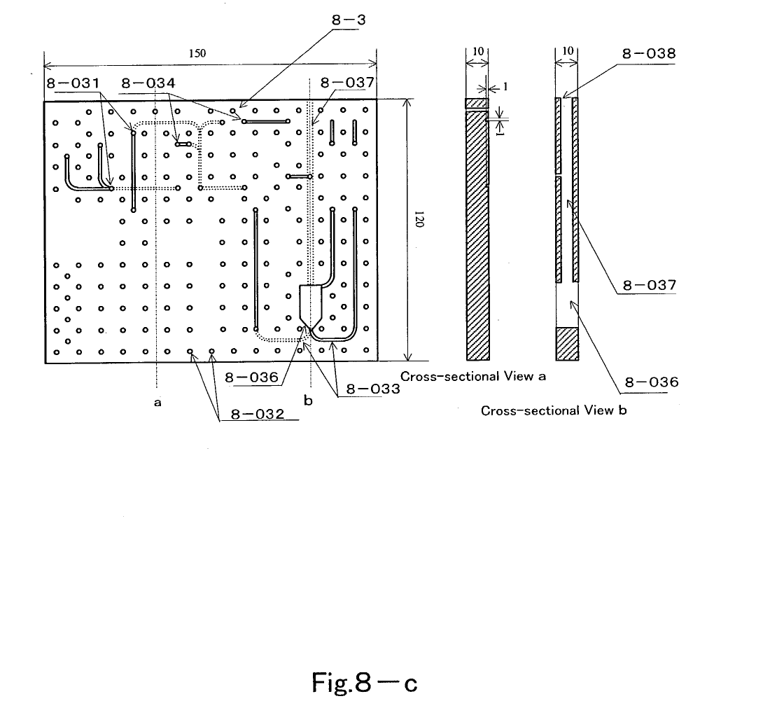

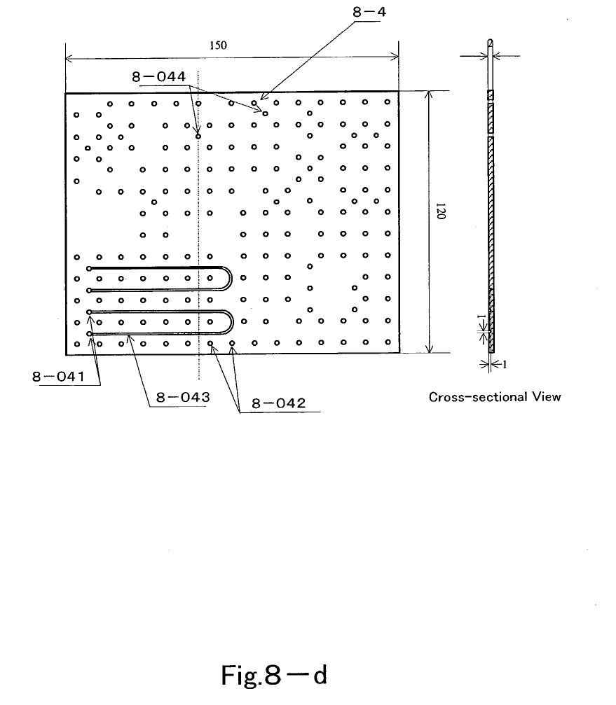

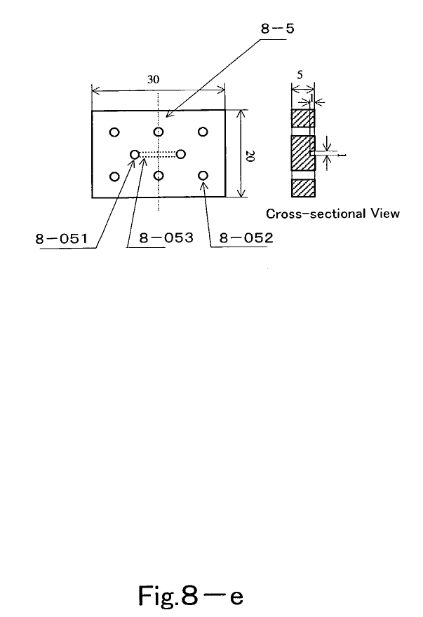

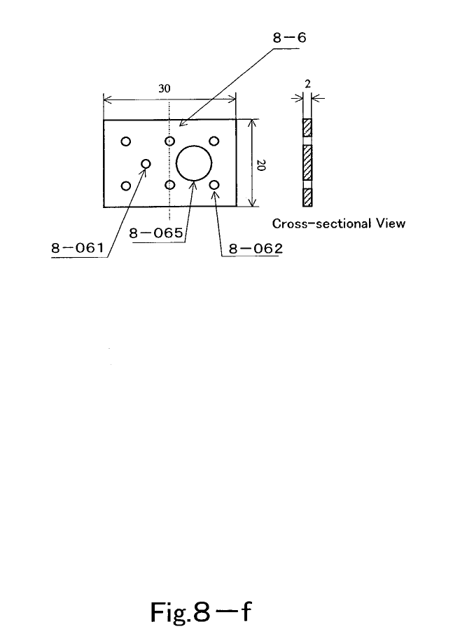

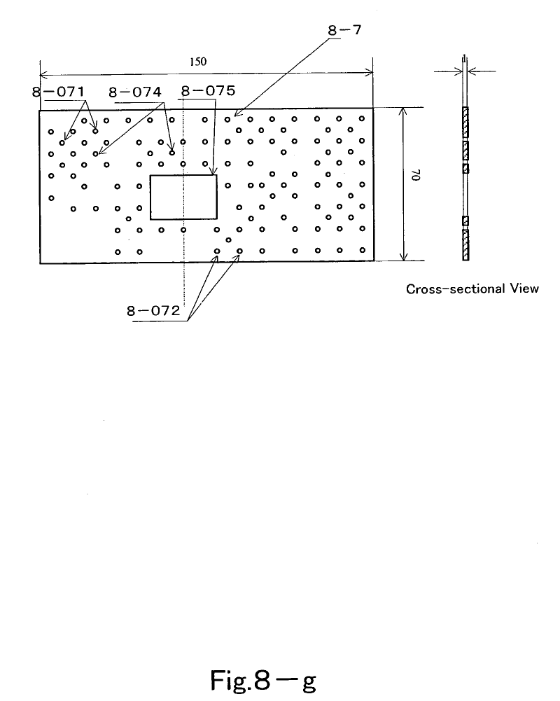

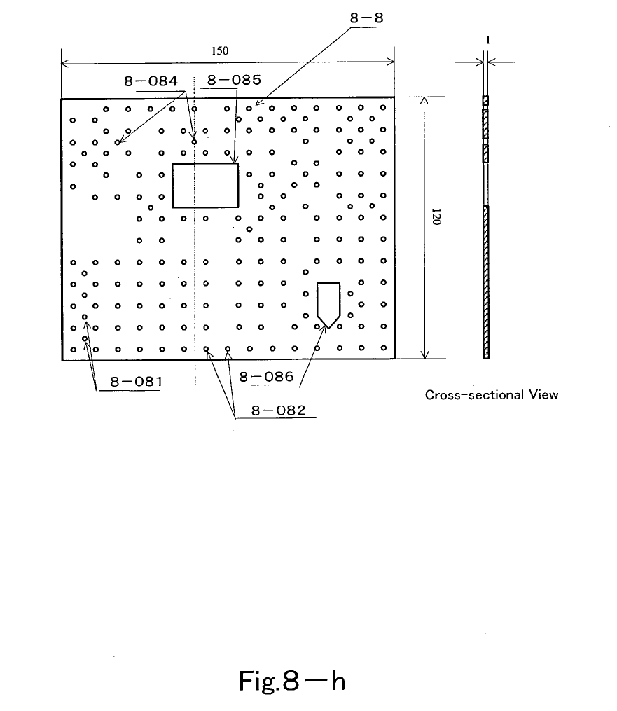

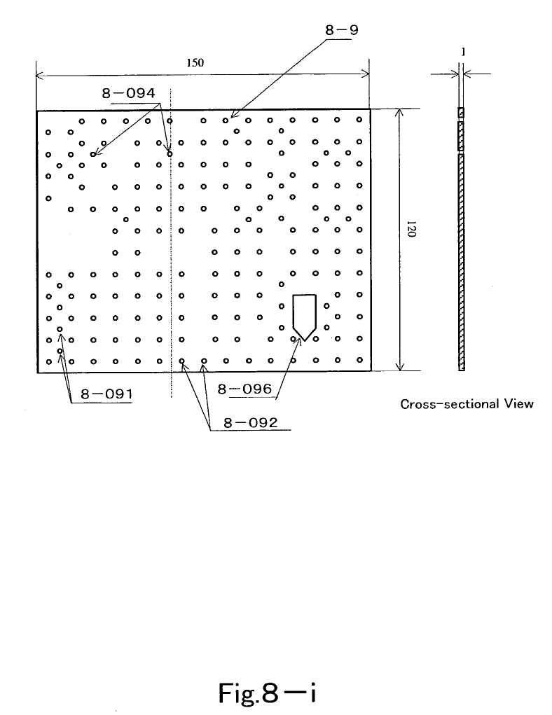

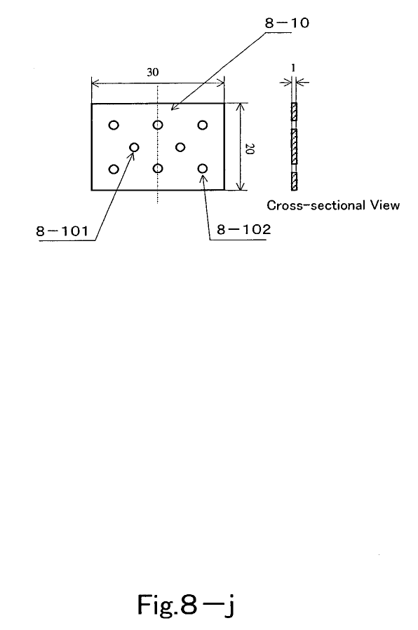

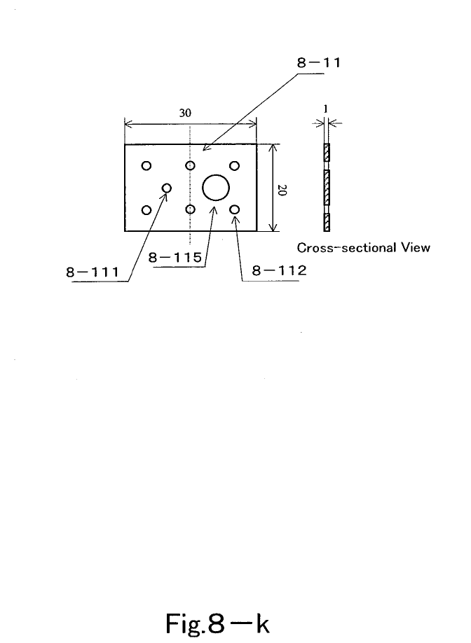

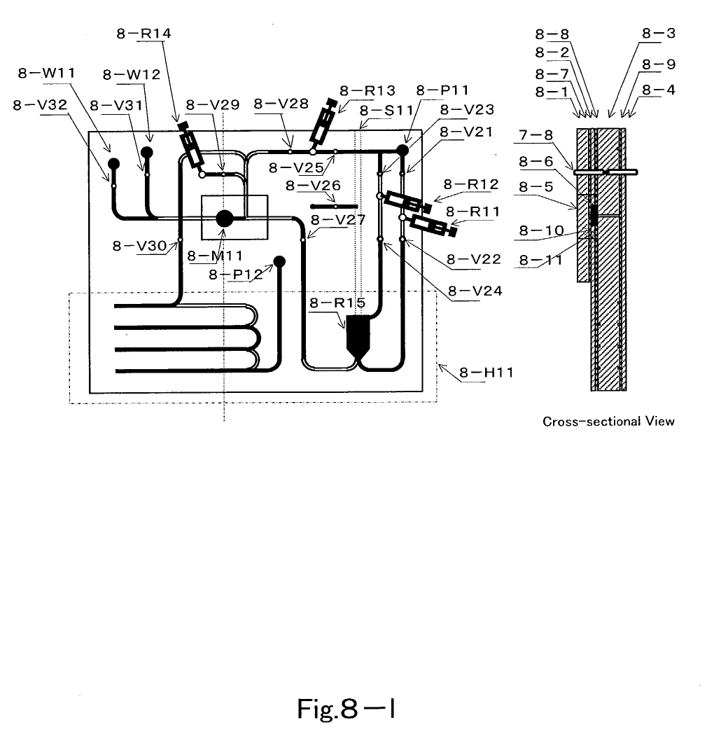

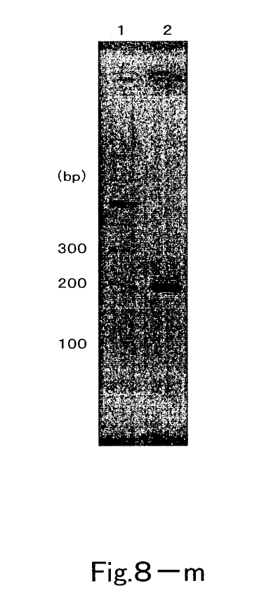

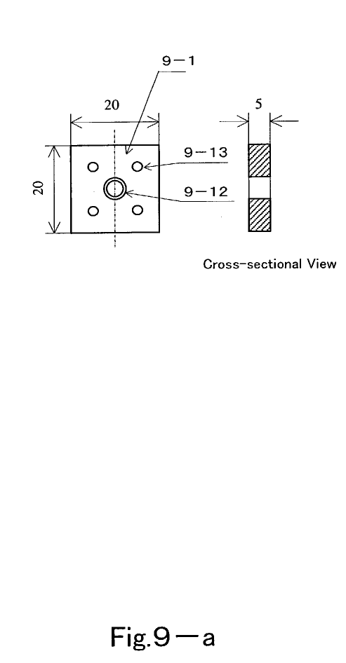

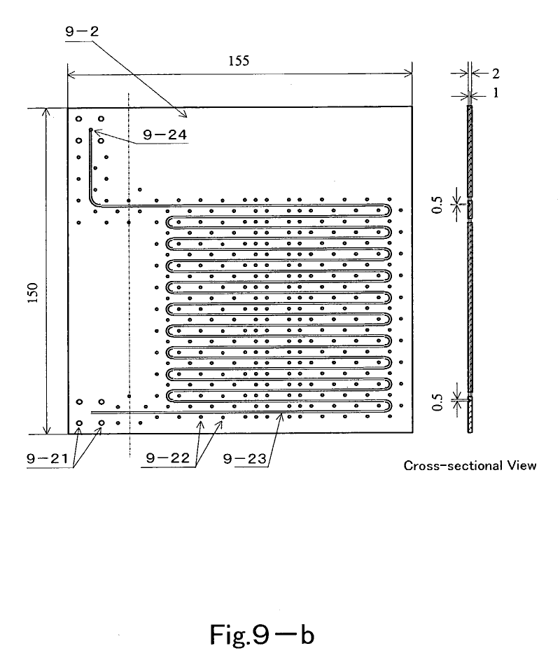

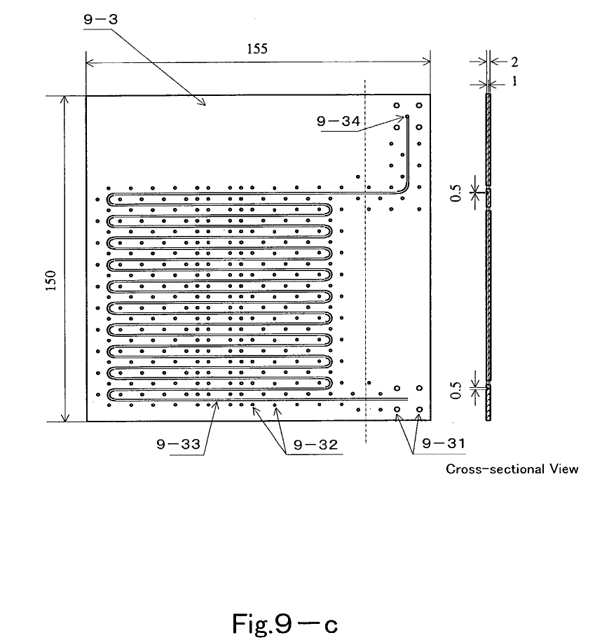

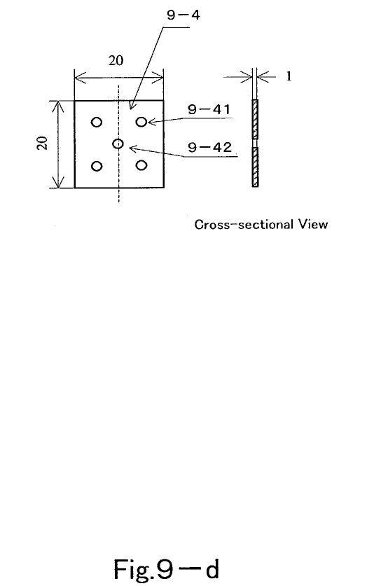

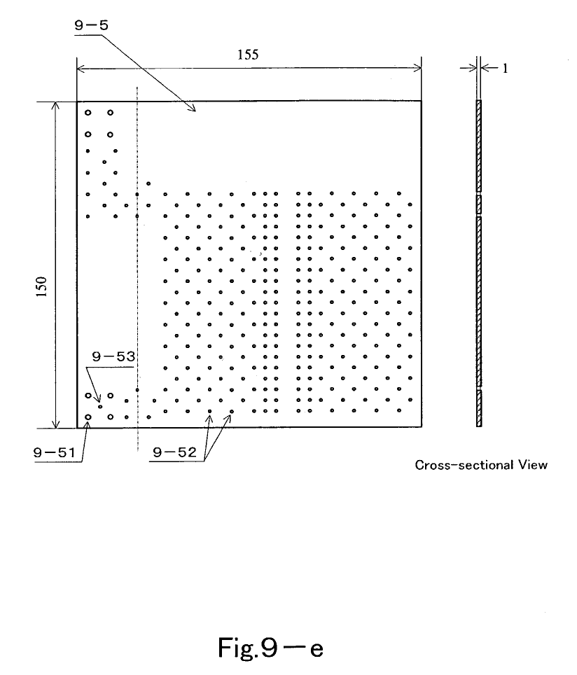

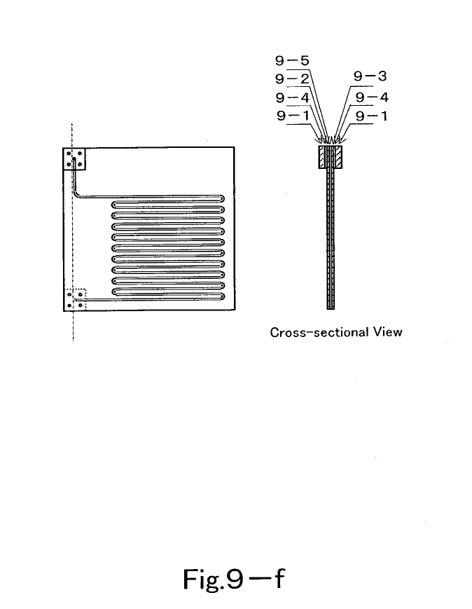

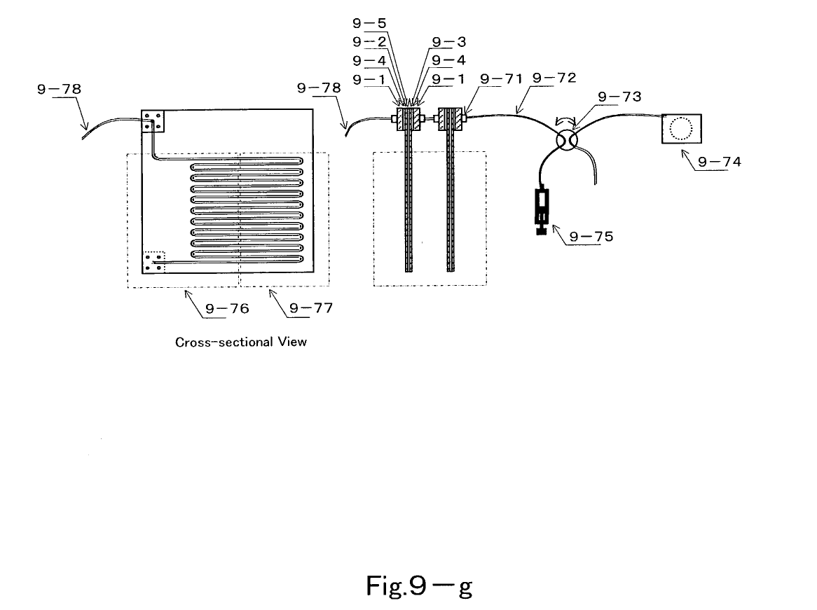

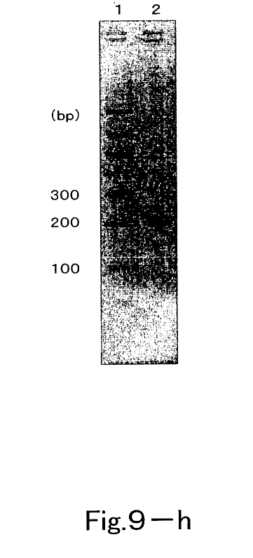

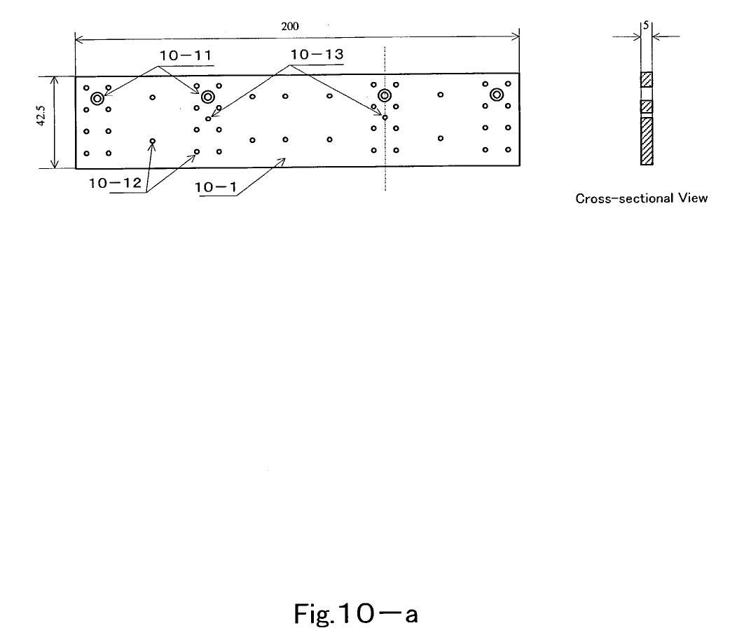

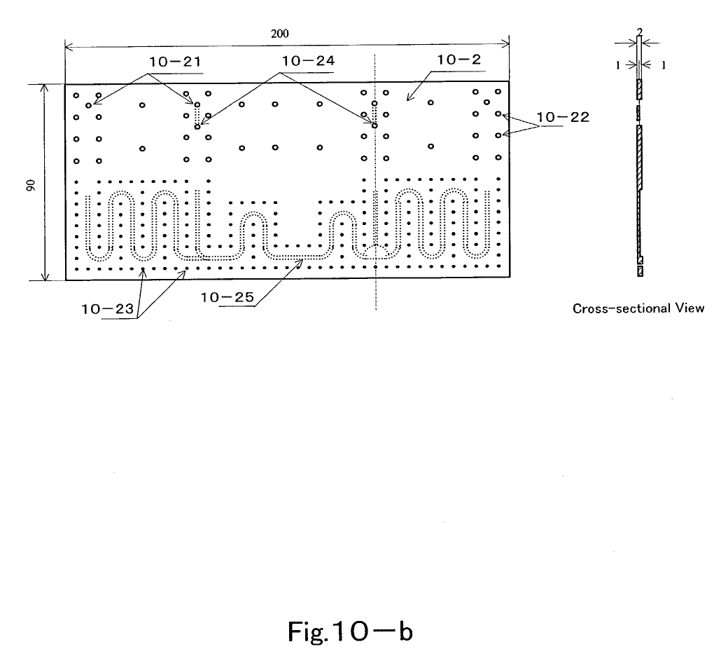

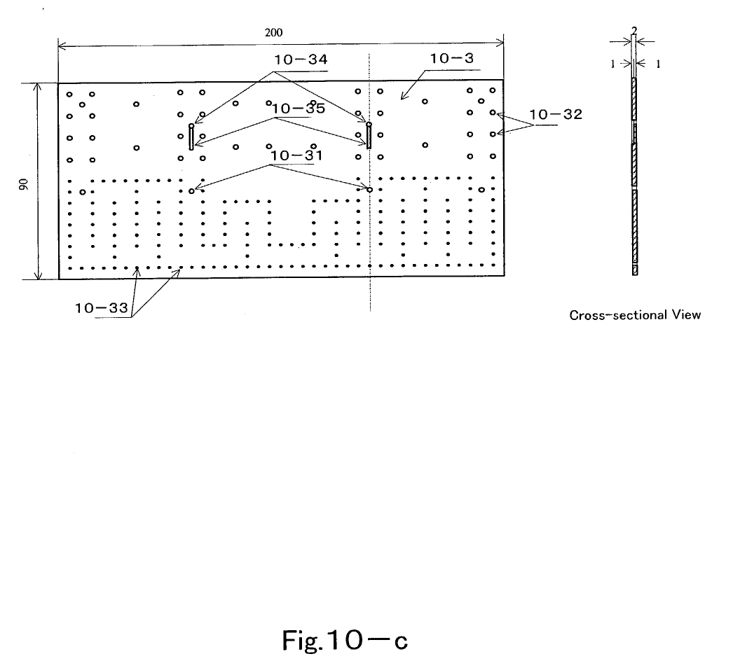

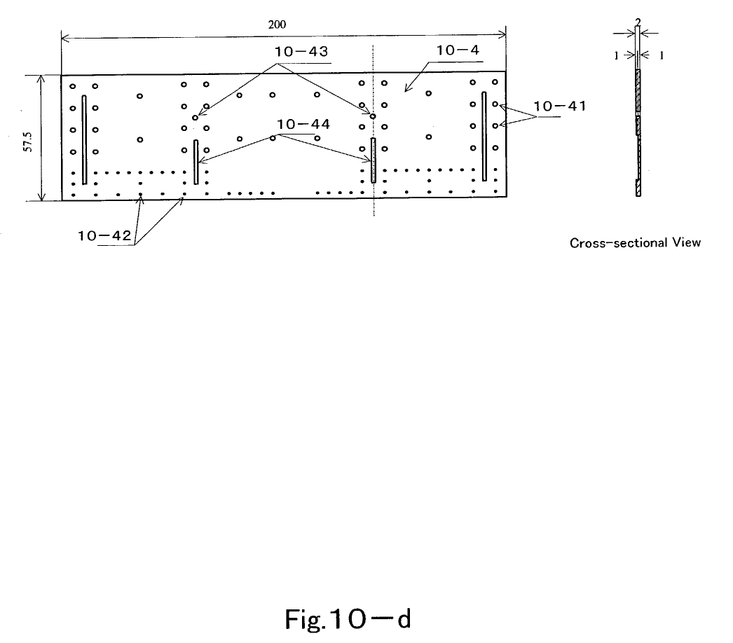

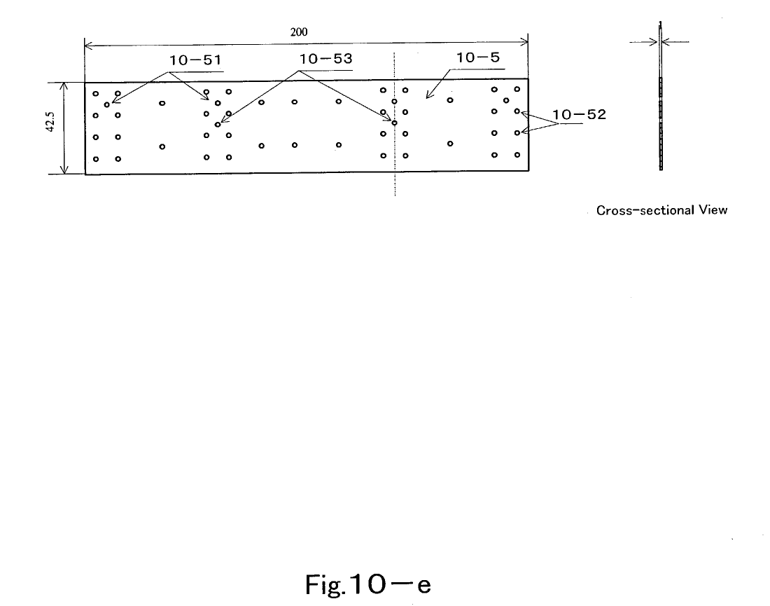

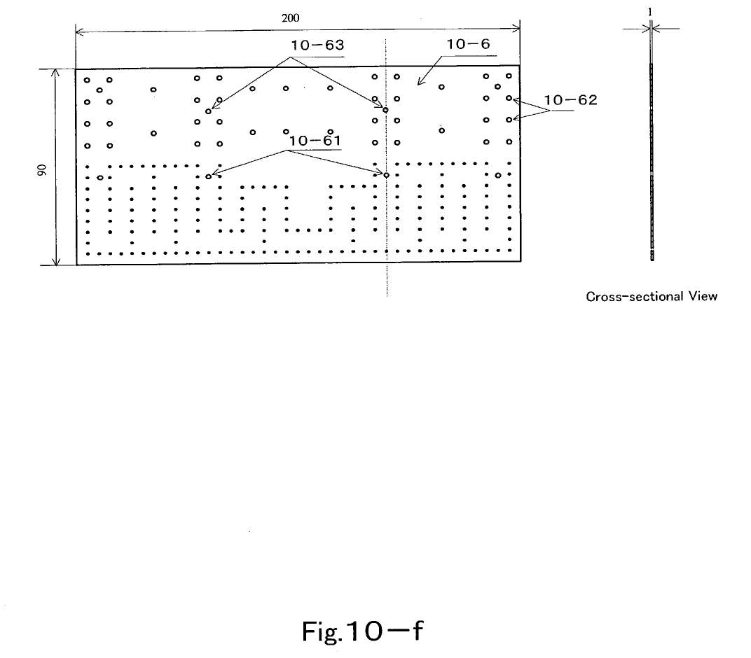

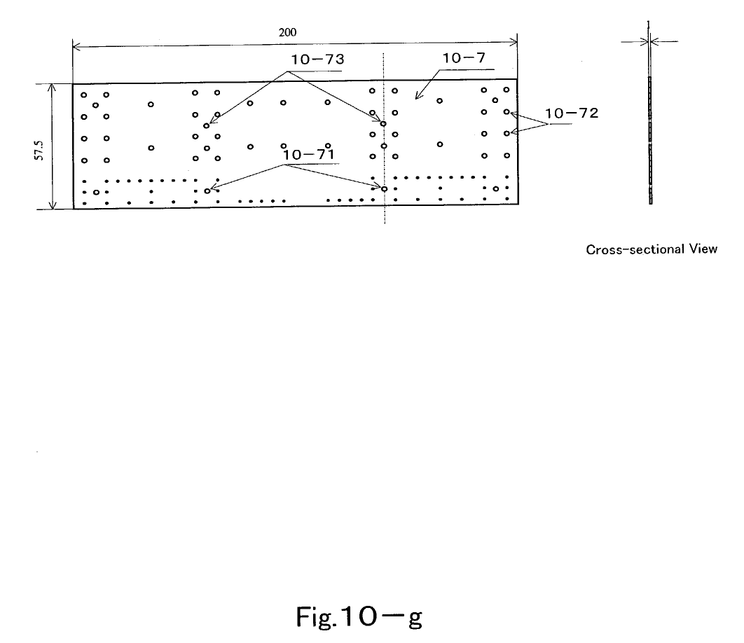

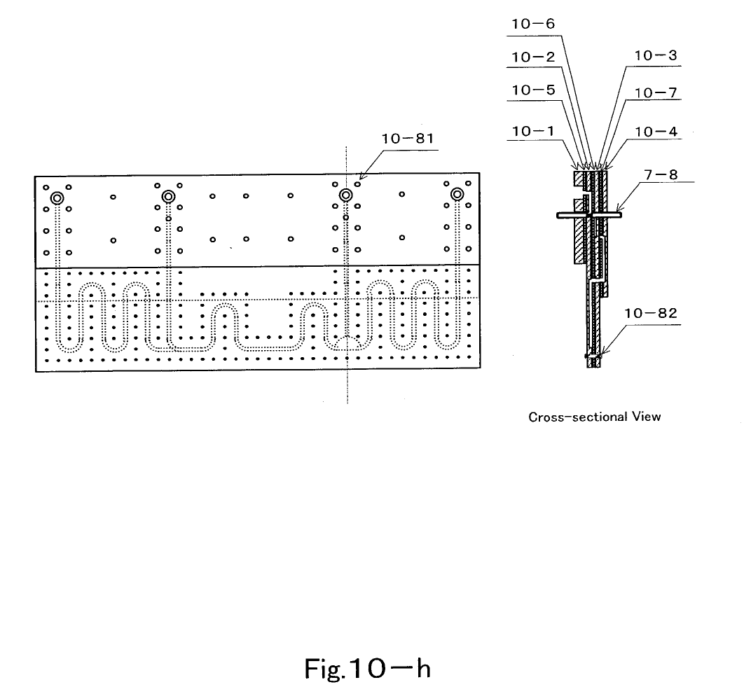

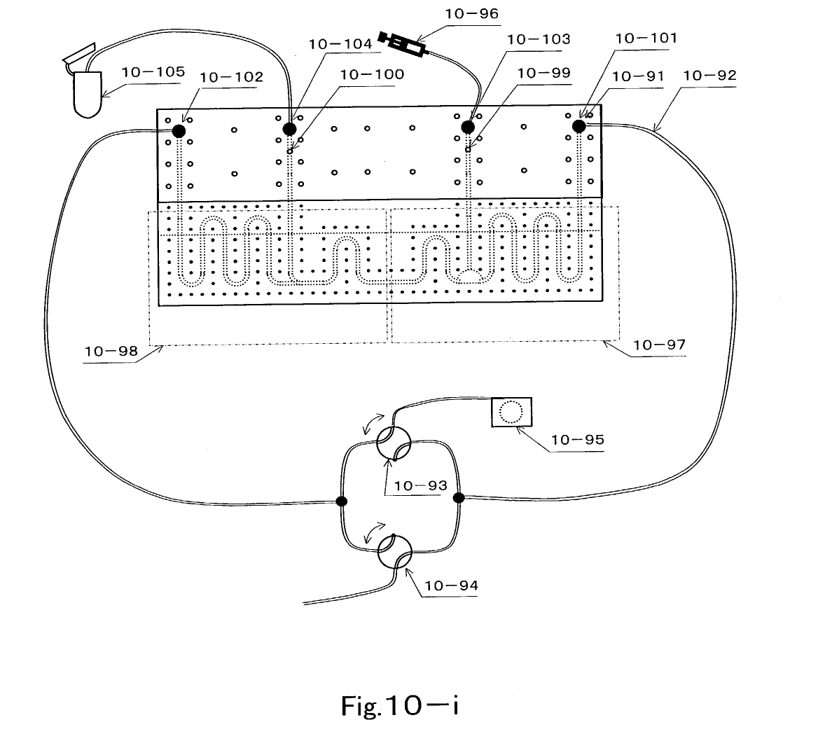

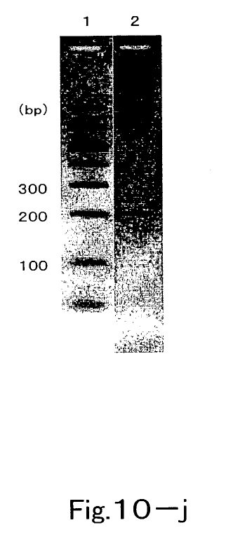

FIG. 1-b shows the result of agarose gel electrophoresis of Example 2. The left margin shows the size of the DNA molecular weight marker (unit: base pair).

Lane 1: DNA molecular weight marker

Lane 2: PCR amplification product of extracted nucleic acid at 98° C.

Lane 3: PCR amplification product of extracted nucleic acid at 90° C.

Lane 4: PCR amplification product of extracted nucleic acid at 80° C.

Lane 5: PCR amplification product of extracted nucleic acid at 70° C.

Lane 6: PCR amplification product of extracted nucleic acid at 60° C.

Lane 7: PCR amplification product of extracted nucleic acid at 50° C.

Lane 8: PCR amplification product of extracted nucleic acid at 40° C.

Lane 9: PCR amplification product of extracted nucleic acid at 30° C. Lane 10: negative control

FIG. 2-a shows the result of agarose gel electrophoresis of Reference Example 1.

Lane 1: DNA molecular weight marker

Lane 2: PCR amplification product under condition of addition of 0.1 μl of guanidine thiocyanate solution

Lane 3: PCR amplification product under condition of addition of 0.2 μl of guanidine thiocyanate solution

Lane 4: PCR amplification product under condition of addition of 0.4 μl of guanidine thiocyanate solution

Lane 5: PCR amplification product under condition of addition of 0.6 μl of guanidine thiocyanate solution

Lane 6: PCR amplification product under condition of addition of 0.8 μl of guanidine thiocyanate solution

Lane 7: PCR amplification product under condition of addition of 1.0 μl of guanidine thiocyanate solution

FIG. 2-b shows the result of agarose gel electrophoresis of Reference Example 2.

Lane 1: DNA molecular weight marker

Lane 2: PCR amplification product under condition of addition of 0.1 μl of 70% ethanol

Lane 3: PCR amplification product under condition of addition of 0.2 μl of 70% ethanol

Lane 4: PCR amplification product under condition of addition of 0.4 μl of 70% ethanol

Lane 5: PCR amplification product under condition of addition of 0.6 μl of 70% ethanol

Lane 6: PCR amplification product under condition of addition of 0.8 μI of 70% ethanol

Lane 7: PCR amplification product under condition of addition of 1.0 μl of 70% ethanol

FIG. 3-a shows a conceptual diagram showing a structure of an apparatus which performs the method for isolating nucleic acid using a flow path for nucleic acid isolation of the present invention as illustrated in Example 3.

FIG. 3-b shows a photo of agarose gel electrophoresis showing the result of Example 3. The left margin shows the size of the DNA molecular weight marker (unit: base pair).

Lane 1: DNA molecular weight marker

Lane 2: amplified DNA fragment

FIG. 4-a shows a conceptual diagram showing a structure of an apparatus which performs the method for isolating and amplifying nucleic acid using a flow path for nucleic acid isolation of the present invention as illustrated in Example 4.

FIG. 4-b shows a photo of agarose gel electrophoresis showing the result of Example 4.

Lane 1: DNA molecular weight marker

Lane 2: amplified DNA fragment

FIG. 5-a shows a horizontal projection and a cross-sectional view of a base plate (5-1) comprising a cartridge of the present invention.

FIG. 5-b shows a horizontal projection and a cross-sectional view of a base plate (5-2) comprising a cartridge of the present invention.

FIG. 5-c shows a horizontal projection and a cross-sectional view of a sealing plate (5-3) comprising a cartridge of the present invention.

FIG. 5-d shows a horizontal projection and a cross-sectional view of a sealing plate (5-4) comprising a cartridge of the present invention.

FIG. 5-e shows a horizontal projection and a cross-sectional view of a rod-shaped element (5-5) comprising a cartridge of the present invention.

FIG. 5-f shows a horizontal projection and a cross-sectional view of a cartridge of the present invention as illustrated in Example 5.

FIG. 6-a shows a horizontal projection and a cross-sectional view of a base plate (6-1) constituting a cartridge of the present invention.

FIG. 6-b shows a horizontal projection and a cross-sectional view of a base plate (6-2) constituting a cartridge of the present invention.

FIG. 6-c shows a horizontal projection and a cross-sectional view of a base plate (6-3) constituting a cartridge of the present invention.

FIG. 6-d shows a horizontal projection and a cross-sectional view of a sealing plate (6-4) constituting a cartridge of the present invention.

FIG. 6-e shows a horizontal projection and a cross-sectional view of a sealing plate (6-5) constituting a cartridge of the present invention.

FIG. 6-f shows a horizontal projection and a cross-sectional view of a rod-shaped element (6-6) constituting a cartridge of the present invention.

FIG. 6-g shows a horizontal projection and a cross-sectional view of a cartridge of the present invention as illustrated in Example 6.

FIG. 7-a shows a horizontal projection and a cross-sectional view of a base plate (7-1) constituting a cartridge for chemical reaction that performs nucleic acid isolation of the present invention.

FIG. 7-b shows a horizontal projection and a cross-sectional view of a base plate (7-2) constituting a cartridge for chemical reaction that performs nucleic acid isolation of the present invention.

FIG. 7-c shows a horizontal projection and a cross-sectional view of a base plate (7-3) constituting a cartridge for chemical reaction that performs nucleic acid isolation of the present invention.

FIG. 7-d shows a horizontal projection and a cross-sectional view of a base plate (7-4) constituting a cartridge for chemical reaction that performs nucleic acid isolation of the present invention.

FIG. 7-e shows a horizontal projection and a cross-sectional view of a sealing plate (7-5) constituting a cartridge for chemical reaction that performs nucleic acid isolation of the present invention.

FIG. 7-f shows a horizontal projection and a cross-sectional view of a sealing plate (7-6) constituting a cartridge for chemical reaction that performs nucleic acid isolation of the present invention.

FIG. 7-g shows a horizontal projection and a cross-sectional view of a sealing plate (7-7) constituting a cartridge for chemical reaction that performs nucleic acid isolation of the present invention.

FIG. 7-h shows a horizontal projection and a cross-sectional view of a rod-shaped element (7-8) constituting a cartridge for chemical reaction that performs nucleic acid isolation of the present invention.

FIG. 7-i shows a horizontal projection and a cross-sectional view of the cartridge for chemical reaction that performs nucleic acid isolation of the present invention as illustrated in Example 7.

FIG. 7-j shows a photo of agarose gel electrophoresis showing the result of Example 7.

Lane 1: DNA molecular weight marker

Lane 2: amplified DNA fragment

FIG. 8-a shows a horizontal projection and a cross-sectional view of a base plate (8-1) constituting a cartridge for chemical reaction that performs nucleic acid isolation of the present invention.

FIG. 8-b shows a horizontal projection and a cross-sectional view of a base plate (8-2) constituting a cartridge for chemical reaction that performs nucleic acid isolation of the present invention.

FIG. 8-c shows a horizontal projection and a cross-sectional view of a base plate (8-3) constituting a cartridge for chemical reaction that performs nucleic acid isolation of the present invention.

FIG. 8-d shows a horizontal projection and a cross-sectional view of a base plate (8-4) constituting a cartridge for chemical reaction that performs nucleic acid isolation of the present invention.

FIG. 8-e shows a horizontal projection and a cross-sectional view of a base plate (8-5) constituting a cartridge for chemical reaction that performs nucleic acid isolation of the present invention.

FIG. 8-f shows a horizontal projection and a cross-sectional view of a base plate (8-6) constituting a cartridge for chemical reaction that performs nucleic acid isolation of the present invention.

FIG. 8-g shows a horizontal projection and a cross-sectional view of a sealing plate (8-7) constituting a cartridge for chemical reaction that performs nucleic acid isolation of the present invention.

FIG. 8-h shows a horizontal projection and a cross-sectional view of a sealing plate (8-8) constituting a cartridge for chemical reaction that performs nucleic acid isolation of the present invention.

FIG. 8-i shows a horizontal projection and a cross-sectional view of a sealing plate (8-9) constituting a cartridge for chemical reaction that performs nucleic acid isolation of the present invention.

FIG. 8-j shows a horizontal projection and a cross-sectional view of a sealing plate (8-10) constituting a cartridge for chemical reaction that performs nucleic acid isolation of the present invention.

FIG. 8-k shows a horizontal projection and a cross-sectional view of a sealing plate (8-11) constituting a cartridge for chemical reaction that performs nucleic acid isolation of the present invention.

FIG. 8-l shows a horizontal projection and a cross-sectional view of a cartridge for chemical reaction that performs nucleic acid isolation of the present invention as illustrated in Example 8.

FIG. 8-m shows the result of agarose gel electrophoresis conducted in Example 8.

Lane 1: DNA molecular weight marker

Lane 2: amplified DNA fragment

FIG. 9-a shows a horizontal projection and a cross-sectional view of a base plate (9-1) constituting a cartridge for chemical reaction that performs the nucleic acid amplification reaction of the present invention.

FIG. 9-b shows a horizontal projection and a cross-sectional view of a base plate (9-2) constituting a cartridge for chemical reaction that performs the nucleic acid amplification reaction of the present invention.

FIG. 9-c shows a horizontal projection and a cross-sectional view of a base plate (9-3) constituting a cartridge for chemical reaction that performs the nucleic acid amplification reaction of the present invention.

FIG. 9-d shows a horizontal projection and a cross-sectional view of a sealing plate (9-4) constituting a cartridge for chemical reaction that performs the nucleic acid amplification reaction of the present invention.

FIG. 9-e shows a horizontal projection and a cross-sectional view of a sealing plate (9-5) constituting a cartridge for chemical reaction that performs the nucleic acid amplification reaction of the present invention.

FIG. 9-f shows a horizontal projection and a cross-sectional view of a cartridge for chemical reaction that performs the nucleic acid amplification reaction of the present invention as illustrated in Example 9.

FIG. 9-g shows a conceptual diagram showing a structure of an apparatus which performs an amplification reaction using a cartridge for chemical reaction that performs the nucleic acid amplification reaction of the present invention as illustrated in Example 9.

FIG. 9-h shows the result of agarose gel electrophoresis of Example 9.

Lane 1: DNA molecular weight marker

Lane 2: amplified DNA fragment

FIG. 10-a shows a horizontal projection and a cross-sectional view of a base plate (10-1) constituting a cartridge for chemical reaction that performs the nucleic acid amplification reaction of the present invention.

FIG. 10-b shows a horizontal projection and a cross-sectional view of a base plate (10-2) constituting a cartridge for chemical reaction that performs the nucleic acid amplification reaction of the present invention.

FIG. 10-c shows a horizontal projection and a cross-sectional view of a base plate (10-3) constituting a cartridge for chemical reaction that performs the nucleic acid amplification reaction of the present invention.

FIG. 10-d shows a horizontal projection and a cross-sectional view of a base plate (10-4) constituting a cartridge for chemical reaction that performs the nucleic acid amplification reaction of the present invention.

FIG. 10-e shows a horizontal projection and a cross-sectional view of a sealing plate (10-5) constituting a cartridge for chemical reaction that performs the nucleic acid amplification reaction of the present invention.

FIG. 10-f shows a horizontal projection and a cross-sectional view of a sealing plate (10-6) constituting a cartridge for chemical reaction that performs the nucleic acid amplification reaction of the present invention.

FIG. 10-g shows a horizontal projection and a cross-sectional view of a sealing plate (10-7) constituting a cartridge for chemical reaction that performs the nucleic acid amplification reaction of the present invention.

FIG. 10-h shows a horizontal projection and a cross-sectional view of a cartridge for chemical reaction that performs the nucleic acid amplification reaction of the present invention as illustrated in Example 10.

FIG. 10-i shows a conceptual diagram showing a structure of an apparatus which performs an amplification reaction using a cartridge for chemical reaction that performs the nucleic acid amplification reaction of the present invention as illustrated in Example 10.

FIG. 10j shows the result of agarose gel electrophoresis of Example 10.

Lane 1: DNA molecular weight marker

Lane 2: amplified DNA fragment

### DESCRIPTION OF THE PREFERRED EMBODIMENTS

Hereinafter, the present invention will be described in more detail with reference to Examples, although the technical scope of the present invention is not limited to these Examples.

### EXAMPLE 1

First, Escherichia coli-derived DNA was purified using a commercially available kit for purifying nucleic acid (Dneasy™ Tissue Kit, Qiagen). A solution (900 μl) for adsorbing/releasing nucleic acid (20 mM EDTA, 1.3% Triton™ X-100, 5.25 M guanidine thiocyanate, 50 mM Tris/HCl buffer solution containing 1 mg/ml of α-casein, pH 6.4) and a solution (10 μl) in which silica particles (SiO₂, (Sigma)) were suspended in distilled water (1 g/ml) were mixed in a polypropylene micro test tube (1.5 ml, Eppendorf), and stirred. To this mixture was added 10 μl of solution containing 10⁻⁴ μg of purified Escherichia coli DNA, and the mixture was then heated for 10 minutes at 30° C., 40° C., 50° C., 60° C., 70° C., 80° C., 90° C., or 98° C. (step 1). Silica particles to which nucleic acid had adsorbed were recovered by centrifugal separation (step 2), 700 μl of washing liquid (70% ethanol) was immediately added thereto, the mixture was suspended, and recovery was performed again by centrifugal separation (step 3). This washing operation was repeated once more. Recovered silica particles to which nucleic acid had adsorbed were dried for 10 minutes at 80° C. (step 4). Thereafter, 50 μl of eluate (TE: 10 mM Tris/HCl, 1 mM EDTA, pH 8.0) was added thereto and the mixture was stirred for 10 minutes to release nucleic acid from the silica particles (step 5), and the supernatant nucleic acid solution was then collected.

The collected eluate (10 μl) was inserted into a microtube for polymerase chain reaction (Multi Ultra PCR Tube, Sorenson™, BioScience), 0.25 μl each of two kinds of 20 mM oligonucleotide solution, 2 μl of substrate solution (solution containing dNTP mixture, dATP, dTTP, dGTP, and dCTP of 2.5 mM respectively (TAKARA)), 0.25 μl of DNA polymerase solution (Z-Taq™, 2.5 U/μl (TAKARA)) and 5 μl of buffer solution (300 mM Tris/HCl, 75 mM ammonium sulfate, 17.5 mM magnesium chloride, pH 8.5) were added thereto, and distilled water was further added to bring the total volume to 25 μl. A polymerase chain reaction of 40 cycles of 98° C. for 20 seconds and 65° C. for 30 seconds was conducted using a thermocycler (RoboCycler™, Stratagene) after overlaying 25 μl of mineral oil (Sigma). As the two kinds of oligonucleotide, the sequence (SEQ ID NO: 1) from the nucleotide 449, c, to the nucleotide 472, t, of the nucleotide sequence of a gene (GenBank D13326) encoding ribosomal protein L25 of Escherichia coli, and the sequence (SEQ ID NO: 2) of the complementary strand of the nucleotide sequence from the nucleotide 628, a, to the nucleotide 650, a, of the same gene, were used. All oligonucleotides shown in the following examples were purchased from Sigma Ltd. as chemically synthesized oligonucleotides. Further, as a negative control, a solution in which distilled water was added in place of eluate was reacted at the same time.

To 5 μl of the solution after polymerase chain reaction was added 0.5 μl of a sample treatment solution (10× Loading Buffer, TAKARA), and this solution was subjected to 3% agarose gel electrophoresis (NuSieve™ 3:1 agarose dissolved and solidified in a TAE (Tris/acetic acid/EDTA) buffer solution) in an electrophoresis tank (Mupid™, ADVANCE Co., Ltd.). After electrophoresis, the agarose gel was immersed for 15 minutes in an ethidium bromide solution (1 μg/ml) and electrophoresed DNA was detected by ultraviolet light. As shown in FIG. 1-a, the results showed that in almost the same position as 200 bp of a DNA molecular weight marker (BioMarker™ Low, BioVentures Inc.) (lane 1), DNA amplified by this polymerase chain reaction (lanes 2 to 9) was electrophoresed as a band of a size to be expected from the nucleotide sequence. From the density of the band it was shown that in a procedure to adsorb nucleic acid on silica particles, at a condition of 80° C. or more (lanes 2 to 4) the polymerase chain reaction was almost completely not inhibited, while at 70 to 60° C. (lanes 5 to 6) some inhibition was observed, and in the case of a condition of 50° C. or below (lanes 7 to 9) the reaction was inhibited.

### EXAMPLE 2

A solution (900 μl) for adsorbing/releasing nucleic acid (20 mM EDTA, 1.3% Triton™ X-100, 5.25 M guanidine thiocyanate, 50 mM Tris/HCl buffer solution containing 1 mg/ml of α-casein, pH 6.4) and magnetic silica particle suspension (10 μl, MagExtractor™, Toyobo Co., Ltd.) were added to a polypropylene micro test tube and stirred. A solution (10 μl) containing 10⁻⁴ μg of Escherichia coli DNA, which was purified in the same manner as in Example 1, was added thereto, and the mixture was heated for 10 minutes at 30° C., 40° C., 50° C., 60° C., 70° C., 80° C., 90° C., or 98° C. (step 1). Magnetic silica particles to which nucleic acid had adsorbed were recovered using a magnet (step 2), 700 μl of 70 % ethanol was immediately added thereto and the mixture suspended, and magnetic silica particles were again recovered using a magnet (step 3). This washing operation was repeated once more. Recovered magnetic silica particles to which nucleic acid had adsorbed were dried for 10 minutes at 80° C. (step 4). Thereafter, 50 μl of H₂O was added thereto and the mixture was stirred for 10 minutes to release nucleic acid from the magnetic silica particles (step 5), and the supernatant nucleic acid solution was then collected.

In the same manner as in Example 1, polymerase chain reaction was conducted using 10 μl of the collected eluate, and electrophoresis and detection were then performed. As shown in FIG. 1-b, the results showed that when a procedure to adsorb nucleic acid on a magnetic silica particle was performed at a condition of 90° C. or more (lanes 2 to 3), in almost the same position as 200 bp of a DNA molecular weight marker (lane 1), DNA amplified by this polymerase chain reaction was observed as a band of a size to be expected from the nucleotide sequence. However, amplified DNA was not observed at a condition of 80° C. or below (lanes 4 to 9). Therefore, it was shown that in a procedure to adsorb nucleic acid on a magnetic silica particle at a condition of 90° C. or more, a polymerase chain reaction was not inhibited.

### Reference Example 1

The mixture solution containing 0.25 μl each of two kinds of 20 mM oligonucleotide solution, 2 μl of substrate solution, 0.25 μl of DNA polymerase solution, and 5 μl of buffer solution (300 mM Tris/HCl, 75 mM ammonium sulfate, 17.5 mM magnesium chloride, pH 8.5) was added to a microtube for polymerase chain reaction (Multi Ultra PCR Tube, Sorenson™, BioScience), and 10⁻⁷ μg of Bacillus subtilis genome DNA was then added thereto. Further, 0.1 μl, 0.2 μl, 0.4 μl, 0.6 μl, 0.8 μl, or 1 μl of guanidine thiocyanate solution (5.25 M guanidine thiocyanate, 50 mM Tris/HCl, pH 6.4) was added thereto, and distilled water was further added to bring the total volume to 25 μl. A polymerase chain reaction of 40 cycles of 98° C. for 20 seconds and 65° C. for 30 seconds was conducted using a thermocycler after overlaying 25 μl of mineral oil. As the two kinds of oligonucleotide, the sequence (SEQ ID NO: 3) from the nucleotide 458, a, to the nucleotide 481, a, of the nucleotide sequence of a gene (GenBank AB018486) encoding 16s rRNA of Bacillus subtilis, and the sequence (SEQ ID NO: 4) of the complementary strand of the nucleotide sequence from the nucleotide 659, t, to the nucleotide 681, c, of the same gene were used.

Using 5 μl of the solution after polymerase chain reaction, electrophoresis and detection were performed in the same manner as in Example 1. As shown in FIG. 2-a, the results showed that in a position between 300 bp and 200 bp of a DNA molecular weight marker (lane 1), DNA amplified by this polymerase chain reaction (lanes 2 to 7) was electrophoresed as a band of a size to be expected from the nucleotide sequence. From the density of the band, it was shown that when 5.25 M guanidine thiocyanate solution is present at 0.8% (0.2 μl) or more of a polymerase chain reaction system, the polymerase chain reaction is inhibited.

### Reference Example 2

The mixture solution containing 0.25 μl each of two kinds of 20 mM oligonucleotide solution, 2 μl of substrate solution, 0.25 μl of DNA polymerase solution, and 5 μl of buffer solution (300 mM Tris/HCl, 75 mM ammonium sulfate, 17.5 mM magnesium chloride, pH 8.5), was added to a microtube for polymerase chain reaction (Multi Ultra PCR Tube, Sorenson™, BioScience), and 10⁻⁷ μg of Bacillus subtilis genome DNA was then added thereto. Further, 0.1 μl, 0.2 μl, 0.4 μl, 0.6 μl, 0.8 μl, or 1 μl of 70% ethanol was added thereto, and distilled water was further added to bring the total volume to 25 μl. In the same manner as in Reference Example 1, polymerase chain reaction was conducted and electrophoresis and detection were then performed. As shown in FIG. 2-b, the results showed that in a position between 300 bp and 200 bp of a DNA molecular weight marker (lane 1), DNA amplified by this polymerase chain reaction (lanes 2 to 7) was electrophoresed as a band of a size to be expected from the nucleotide sequence. From the density of the band, it was shown that when 70% ethanol is present at 1.6% (0.4 μl) or more of a polymerase chain reaction system, the reaction is inhibited.

### EXAMPLE 3

Reservoirs 3-1 to 3-4 and a reaction chamber (3-5) were made using a polypropylene micro test tube and a silicon rubber plug, valves 3-6 to 3-9 were made using a magnetic valve (IMV-8, Pharmacia, (now, Amersham BioScience)), traps 3-10 to 3-12 were made using a Teflon tube (outer diameter 2.5 mm, inner diameter 1.5 mm), and a flow path connecting each part was made with a Teflon™ tube (outer diameter 1.7 mm, inner diameter 0.9 mm), to complete a flow path for nucleic acid isolation as shown in FIG. 3-a. A connector of a tube (Pharmacia) used in liquid chromatography was used as a joint with each part. A mixed solution of 900 μl of a solution for adsorbing/releasing nucleic acid (20 mM EDTA, 1.3% Titon™ X-100, 50 mM Tris/HCl buffer solution containing 5.25 M guanidine thiocyanate, pH 6.4) and 1 μl of magnetic silica particle suspension was added to reservoir 3-1. A washing liquid (500 μl, 70% ethanol) was added to reservoir 3-2 and reservoir 3-3, respectively. Distilled water (100 μl) was added to reservoir 3-4 as an eluate. To this apparatus were respectively mounted an air pump (3-13) to supply air (diverted from P-6000, Pharmacia), an apparatus (3-14) to heat the reaction chamber (3-5), a valve controller (LCC-500, Pharmacia) (3-15), and cylindrical magnets 3-16 to 3-18 (diameter 8 mm, length 25 mm) as devices to impart a magnetic field to the part of traps 3-10 to 3-12. Previously, the air pump (3-13) was adjusted such that a flow rate at a time of supplying air was 10 ml/min and a maximum pressure was 0.1 atm, and valves 3-6 to 3-9 were all closed.

A Bacillus subtilis pellet (including 10⁷ cells) was inserted into the reaction chamber (3-5), and this part was heated to 98° C. by the heater (3-14). After opening valve 3-6, reservoir 3-1 was supplied with air from the air pump (3-13), and a solution for adsorbing/releasing nucleic acid was introduced to the reaction chamber (3-5). After 10 minutes, valve 3-6 was closed and valve 3-7 and valve 3-8 were opened, and when the content of the reaction chamber (3-5) was discharged to a waste fluid outlet (3-19), magnetic silica particles were accumulated at the part of trap 3-10. After discharge, magnet 3-16 that was mounted at trap 3-10 was removed, air was supplied to reservoir 3-2 by the air pump (3-13), and magnetic silica particles accumulated in trap 3-10 were dispersed and washed with washing liquid, and at the same time, the magnetic silica particles were accumulated once more at the part of trap 3-11 and washing liquid was discharged to the waste fluid outlet (3-19). After discharge, magnet 3-17 that was mounted at trap 3-11 was removed, valve 3-7 was closed, air was supplied to reservoir 3-3 by the air pump (3-13), and magnetic silica particles accumulated in trap 3-11 were dispersed and washed with washing liquid, and at the same time, the magnetic silica particles were accumulated once more at the part of trap 3-12 and washing liquid was discharged to the waste fluid outlet (3-19). After discharge, valve 3-7 was closed and valve 3-9 was opened, magnet 3-18 that was mounted at trap 3-12 was removed, air was supplied to reservoir 3-4 by the air pump (3-13), and magnetic silica particles accumulated in trap 3-12 were recovered from the recovery outlet (3-20) together with eluate.

Using 10 μl of the recovered eluate, polymerase chain reaction, electrophoresis and detection were performed in the same manner as in Example 1. As the two kinds of oligonucleotide, the sequence (SEQ ID NO: 3) from the nucleotide 458, a, to the nucleotide 481. a, of the nucleotide sequence of a gene encoding 16s rRNA of Bacillus subtilis, and the sequence (SEQ ID NO: 4) of the complementary strand of the nucleotide sequence from the nucleotide 659, t, to the nucleotide 681, c, of the same gene were used. As shown in FIG. 3-b, the results showed that in a position between 300 bp and 200 bp of a DNA molecular weight marker (lane 1), DNA amplified by this polymerase chain reaction (lane 2) was electrophoresed as a band of a size to be expected from the nucleotide sequence. Thus, it was shown that DNA of Bacillus subtilis was isolated by this technique.

### Comparative Example 1

The part of trap 3-11 and trap 3-12 of the apparatus constructed in Example 3 was removed, and an apparatus was assembled in which this part was connected by a flow path. In the same manner as in Example 3, 1 μl of magnetic silica particle suspension was added to 900 μl of a solution for adsorbing/releasing nucleic acid (20 mM EDTA, 1.3% Triton™ X-100, 50 mM Tris/HCl buffer solution comprising 5.25 M guanidine thiocyanate, pH 6.4) in reservoir 3-1. Washing liquid (500 μl, 70% ethanol) was added to reservoir 3-2 and reservoir 3-3, respectively. Distilled water (100 μl) was added to reservoir 3-4 as an eluate. To this apparatus were respectively mounted an apparatus (3-13) for feeding fluid by means of an air pump, an apparatus (3-14) for heating the reaction chamber (3-5), a valve controller (3-15), and a cylindrical magnet 3-16 (diameter 8 mm, length 25 mm) as an apparatus to impart a magnetic field to the trap 3-10 part. Previously, the air pump (3-13) was adjusted such that a flow rate at a time of supplying air was 10 ml/min and a maximum pressure was 0.1 atm, and valves 3-6 to 3-9 were all closed.

A Bacillus subtilis pellet (including 10⁷ cells) was inserted into the reaction chamber (3-5), and this part was heated to 98° C. by the heater (3-14). After opening valve 3-6, air was supplied to reservoir 3-1 from the air pump (3-13), and releasing and adsorbing solution was thus introduced to the reaction chamber (3-5). After 10 minutes, valve 3-6 was closed and valve 3-7 and valve 3-8 were opened, and the content of the reaction chamber (3-5) was discharged to a waste fluid outlet (3-19). After discharge, air was supplied to reservoir 3-2 by the air pump (3-13), magnetic silica particles accumulated in trap 3-10 were washed, and washing liquid was discharged to the waste fluid outlet (3-19). After discharge, valve 3-7 was closed, air was supplied to reservoir 3-3 by the air pump (3-13), magnetic silica particles accumulated in trap 3-10 were washed, and washing liquid was discharged to the waste fluid outlet (3-19). After discharge, valve 3-8 was closed and valve 3-9 was opened, magnet (3-16) that was mounted at trap 3-10 was removed, air was supplied to reservoir 3-4 by the air pump (3-13), and it was attempted to recover magnetic silica particles that had accumulated in trap 3-10 from the recovery outlet (3-20) together with eluate. At this time, magnetic silica particles that had accumulated in trap 3-10 did not disperse but formed a mass that flowed through the flow path and blocked the connecting part of the valve and tube, and thus eluate comprising nucleic acid could not be recovered from the recovery outlet.

### EXAMPLE 4

Reservoirs 4-1 to 4-3 and a reaction chamber (4-4) were constructed with a polypropylene micro test tube and a silicon rubber plug, a reservoir 4-5 was constructed using a sample injector (IMV-7, sample loop volume 50 μl, Pharmacia), valves 4-6 to 4-9 were constructed with a magnetic valve (IMV-8, Pharmacia), and traps 4-10 to 4-12 were constructed with a Teflon™ tube (outer diameter 2.5 mm, inner diameter 1.5 mm). To this apparatus were mounted an apparatus (4-13) for feeding fluid by an air pump, a heater (4-14) to heat the reaction chamber (4-4), a valve controller (4-15), and cylindrical magnets 4-16 to 4-18 (diameter 8 mm, length 25 mm) which were mounted at the part of traps 4-10 to 4-12. Further, a flow path connecting each part was constructed with a Teflon™ tube (outer diameter 1.7 mm, inner diameter 0.9 mm). In addition, a fluid pump (P-500, Pharmacia) (4-20) for conducting a polymerase chain reaction, a heater 4-21 to heat the trap part, and heater 4-22 and 4-23 to heat parts for conducting a polymerase chain reaction to two differing temperatures were respectively provided. A flow path (Teflon™ tube, outer diameter 1.7 mm, inner diameter 0.5 mm, 1 cycle of back and forth comprising a length of 20 cm) was mounted running back and forth 40 times over parts heated to the two differing temperatures, to thus complete the apparatus shown in FIG. 4-a. A connector of a tube used in liquid chromatography was used as a joint between the flow path and each part.

To reservoir 4-1 was added 900 μl of a solution for adsorbing/releasing nucleic acid (20 mM EDTA, 1.3% Triton™ X-100, 50 mM Tris/HCl buffer solution comprising 5.25 M guanidine thiocyanate, pH 6.4) and 1 μl of magnetic silica particle suspension. Washing liquid (500 μl, 70% ethanol) was added to reservoir 4-2 and reservoir 4-3. A solution (50 μl) for conducting a polymerase chain reaction (solution comprising 0.125 U DNA polymerase, 3.5 mM magnesium chloride, two kinds of oligonucleotide (SEQ ID NOS: 3 and 4) of 0.2 mM each, and substrates (0.2 mM each dATP, dTTP, dGTP, dCTP)) was added to reservoir 4-5 as an eluate. Previously, air pump (4-13) was adjusted such that a flow rate at a time of supplying air was 10 ml/min and a maximum pressure was 0.1 atm, and valves 4-6 to 4-9 were all closed.

A Bacillus subtilis pellet (including 10⁷ cells) was inserted to the reaction chamber (4-4), and this part was heated to 98° C. by heater 4-14. After opening valve 4-6, air was supplied to reservoir 4-1 from the air pump (4-13) to introduce a solution for adsorbing/releasing nucleic acid to the reaction chamber (4-4). After 10 minutes, valve 4-6 was closed and valve 4-7 and valve 4-8 were opened, and when the solution of the reaction chamber was discharged from the waste fluid outlet (4-19), magnetic silica particles were accumulated in trap 4-10. Magnet 4-16 that was mounted at trap 4-10 was removed, air was supplied to reservoir 4-2 by the air pump, and magnetic silica particles accumulated in trap 4-10 were dispersed and washed with washing liquid, and at the same time, the magnetic silica particles were accumulated once more at the part of trap 4-11 and washing liquid was discharged. After discharge, magnet 4-17 that was mounted at trap 4-11 was removed, valve 4-7 was closed, air was supplied to reservoir 4-3 by the air pump (4-13), and magnetic silica particles accumulated in trap 4-11 were dispersed and washed with washing liquid, and at the same time, the magnetic silica particles were accumulated again at the part of trap 4-12 and washing liquid was discharged. The flow rate of the air pump (4-13) was set to 50 ml/min, the trap part was heated to approximately 90° C. by heater 4-21, and magnetic silica particles accumulated in trap 4-12 were sufficiently dried. After drying, valve 4-8 was closed and valve 4-9 was opened, magnet 4-18 that was mounted at trap 4-12 was removed, and mineral oil was fed to reservoir 4-5 at a flow rate of 0.12 ml/min using the liquid pump (4-20).

The polymerase chain reaction parts were heated to 92° C. and 65° C., respectively, by heater 4-22 and 4-23, and polymerase chain reaction was conducted in a PCR reaction flow path (4-24). From the recovery outlet (4-25), a solution in which polymerase chain reaction was completed was recovered. Using 5 μl of the recovered solution, electrophoresis and detection were performed in the same manner as in Example 1. As shown in FIG. 4-b, the results showed that in a position between 300 bp and 200 bp of a DNA molecular weight marker (lane 1), DNA amplified by this polymerase chain reaction (lane 2) was electrophoresed as a band of a size to be expected from the sequence. Thus, it was shown that Bacillus subtilis-derived DNA was isolated and, furthermore, amplified by this technique.

### EXAMPLE 5

A base plate (5-1) shown in FIG. 5-a was made with a polycarbonate plate of 50 mm in length, 50 mm in width and a thickness of 5 mm. Into the base plate were worked a 6-mm female screw hole (5-11) provided such that a connector of a tube used in liquid chromatography can be inserted therein; a hole (5-12) of 3 mm in diameter having a clearance such that a rod-shaped element (5-5) is movable when inserted therein; and holes (5-13) of 2 mm in diameter penetrable by a 2-mm stainless steel pan-head screw used in crimping. A base plate (5-2) shown in FIG. 5-b was made with a polycarbonate plate of 50 mm in length, 50 mm in width and a thickness of 2 mm. Into the base plate were worked a hole (5-21) of 1 mm in diameter in a position corresponding to the 6-mm female screw hole (5-11) of base plate 5-1 as a flow path; a hole (5-22) of 3 mm in diameter having a clearance such that a rod-shaped element (5-5) is movable when inserted therein; a groove (5-24) of a depth of 1 mm and a width of 1 mm as a flow path connecting to these holes; and holes (5-23) of 2 mm in diameter penetrable by a 2-mm stainless steel pan-head screw used in crimping. A sealing plate (5-3) shown in FIG. 5-c was made with a silicon rubber plate of 50 mm in length, 50 mm in width and a thickness of 1 mm. Into the sealing plate were worked a hole (5-31) of 1 mm diameter in a position corresponding to the 6-mm female screw hole (5-11) of base plate 5-1 as a flow path; a hole (5-32) of 3 mm in diameter having a clearance such that a rod-shaped element (5-5) is movable when inserted therein, and provided such that when a part of the rod-shaped element having no notch is inserted therein, hermeticity is maintained; and holes (5-33) of 2 mm in diameter penetrable by a 2-mm stainless steel pan-head screw used in crimping. A sealing plate (5-4) shown in FIG. 5-d was made with a silicon rubber plate of 50 mm in length, 50 mm in width and a thickness of 1 mm. Into the sealing plate were worked a hole (5-42) of 3 mm in diameter having a clearance such that a rod-shaped element (5-5) is movable when inserted therein, and provided such that when a part of the rod-shaped element having no notch is inserted therein, hermeticity is maintained; and holes (5-43) of 2 mm in diameter penetrable by a 2-mm stainless steel pan-head screw used in crimping. A rod-shaped element (5-5) shown in FIG. 5-e was made with a Teflon™ rod of 3 mm in diameter and 20 mm in length. As shown in the figure, in the vicinity of the center of the rod, a notch (5-51) of 2 mm in width and 1 mm depth was worked into the rod-shaped element in the lengthwise direction. These parts were crimped in a multilayered shape using a set (5-6) of a stainless steel pan-head screw (diameter 2 mm, length 20 mm) and nut, to thus construct the cartridge shown in FIG. 5-f.

Using a connector of a tube used in liquid chromatography, a Teflon™ tube (inner diameter 0.9 mm, outer diameter 1.7 mm) was connected to each of the two sides of this cartridge, and one end of each tube was connected to an air pump. Air adjusted to have a maximum pressure of 0.1 atm was supplied to this cartridge at a flow rate of 10 ml/min. Results showed that no leakage of air was confirmed from a flow path of this cartridge, and further, by movement of the rod-shaped element (5-5), an open state and closed state of a flow path inside the cartridge was attained. Specifically, when the rod-shaped element was moved to be positioned such that the notch part (5-51) of the rod-shaped element communicated with a flow path formed by the two base plates 5-2 and the one sealing plate 5-4, the valve opened, and when the rod-shaped element was moved to the position of base plate 5-1 such that the notch part (5-51) did not communicate with a flow path formed by the two base plates 5-2 and the one sealing plate 5-4, the valve closed.

Accordingly, it was confirmed that this cartridge functioned as a flow path comprising a valve capable of opening and closing. Also, as the sealing plates (5-3, 5-4) have greater elasticity than the base plates (5-1, 5-2), principally the sealing plates (5-3, 5-4) change shape slightly in accordance with adjustment of the crimping force of the screw and nut (5-6), and thus the level of adherence between the sealing plates (5-3, 5-4) and the base plates (5-1, 5-2) and the level of clearance between the rod-shaped element (5-5) and the sealing plates (5-3, 5-4) can be adjusted. By such adjustment, it is possible to adjust the hermeticity of a flow path or valve part on a cartridge.

### EXAMPLE 6

A base plate (6-1) shown in FIG. 6-a was made with a polycarbonate plate of 50 mm in length, 50 mm in width and a thickness of 5 mm. Into the base plate were worked a 6-mm female screw hole (6-11) provided such that a connector of a tube used in liquid chromatography can be inserted therein, and holes (6-12) of 2 mm in diameter penetrable by a 2-mm stainless steel pan-head screw used in crimping. A base plate (6-2) shown in FIG. 6-b was made with a polycarbonate plate of 50 mm in length, 50 mm in width and a thickness of 2 mm. Into the base plate were worked a hole (6-21) of 1 mm in diameter in a position corresponding to a 6-mm female screw hole (6-31) of base plate 6-3 as a flow path; holes (6-22) of 2 mm in diameter penetrable by a 2-mm stainless steel pan-head screw used in crimping; holes (6-23) of 1 mm in diameter in two places as flow paths; and grooves (6-24) of a depth of 1 mm and a width of 1 mm in two places as flow paths. A base plate (6-3) shown in FIG. 6-c was made with a polycarbonate plate of 50 mm in length, 50 mm in width and a thickness of 5 mm. Into the base plate were worked a 6-mm female screw hole (6-31) provided such that a connector of a tube used in liquid chromatography can be inserted therein; holes (6-32) of 2 mm in diameter penetrable by a 2-mm stainless steel pan-head screw used in crimping; a hole (6-33) of 3 mm in diameter having a clearance such that a rod-shaped element (6-6) is movable when inserted therein; and a groove (6-34) of a width of 3 mm and a depth of 2 mm connecting to this hole. A sealing plate (6-4) shown in FIG. 6-d was made with a silicon rubber plate of 50 mm in length, 50 mm in width and a thickness of 1 mm. Into the sealing plate were worked a hole (6-41) of 1 mm diameter in a position corresponding to the 6-mm female screw hole (6-11) of base plate 6-1 as a flow path, and holes (6-42) of 2 mm in diameter penetrable by a 2-mm stainless steel pan-head screw used in crimping. A sealing plate (6-5) shown in FIG. 6-e was made with a silicon rubber plate of 50 mm in length, 50 mm in width and a thickness of 1 mm. Into the sealing plate were worked a hole (6-51) of 1 mm in diameter in a position corresponding to the 6-mm female screw hole (6-31) of base plate 6-3 as a flow path, and holes (6-52) of 2 mm in diameter penetrable by a 2-mm stainless steel pan-head screw used in crimping. A rod-shaped element (6-6) shown in FIG. 6-f was made with a Teflon™ rod of 3 mm in diameter and 20 mm in length.

These parts were crimped in a multilayered shape using sets (6-7) of a stainless steel pan-head screw (diameter 2 mm, length 20 mm) and nut, to construct the cartridge shown in FIG. 6-g. Using a connector of a tube used in liquid chromatography, a Teflon™ tube (inner diameter 0.9 mm, outer diameter 1.7 mm) was connected to each of the two sides of this cartridge, and one end of each tube was connected to an air pump. Air adjusted to have a maximum pressure of 0.1 atm was supplied to this cartridge at a flow rate of 10 ml/min. Results showed that no leakage of air was confirmed from the flow path of this cartridge, and further, in accordance with the presence or absence of a force pressing the rod-shaped element (6-6) in the direction of a flow path, an open state or closed state of the flow path inside the cartridge was achieved. Thus, it was confirmed that this cartridge functioned as a flow path comprising a valve capable of opening and closing. More specifically, for this valve, in a case of no force pressing the rod-shaped element, by the pressure of air flowing in a flow path, in sealing plate 6-5, a part contacting a groove (6-34) of base plate 6-3 changes shape in the direction of the groove and thus the flow path is maintained, resulting in an open state, and in a case when the rod-shaped element is pressed in the direction of a flow path, sealing plate 6-5 is pressed against base plate 6-2 and therefore the flow path is not maintained, resulting in a closed state. Moreover, as the sealing plates (6-4, 6-5) have greater elasticity than the base plates (6-1, 6-2, 6-3), principally the sealing plates (6-4, 6-5) change shape slightly in accordance with adjustment of the crimping force of the screw and nut (6-7), and therefore the level of adherence between the sealing plates (6-4, 6-5) and the base plates (6-1 to 6-3) can be adjusted. By such adjustment, it is possible to adjust the hermeticity of a flow path or valve part on a cartridge.

### EXAMPLE 7

A base plate (7-1) shown in FIG. 7-a was made with a polycarbonate plate of 70 mm in length, 150 mm in width and a thickness of 5 mm. Into the base plate were worked 6-mm female screw holes (7-11) provided such that a connector of a tube used in liquid chromatography can be inserted therein; holes (7-12) of 2 mm in diameter penetrable by a 2-mm stainless steel pan-head screw used in crimping; holes (7-13) of 4 mm in diameter provided such that a plastic syringe can be inserted therein (all syringes used hereinafter are manufactured by Terumo); holes (7-14) of 2 mm in diameter having a clearance such that a rod-shaped element (7-8) is movable when inserted therein; and elliptic holes (7-15) provided such that two cylindrical magnets of 5 mm in diameter and 4 mm in length for a trap can be aligned and inserted therein. A base plate (7-2) shown in FIG. 7-b was made with a polycarbonate plate of 120 mm in length, 150 mm in width and a thickness of 2 mm. Into the base plate were worked holes (7-22) of 2 mm in diameter penetrable by a 2-mm stainless steel pan-head screw used in crimping; holes (7-24) of 2 mm in diameter having a clearance such that a rod-shaped element (7-8) is movable when inserted therein; grooves (7-23) of a width of 1 mm and a depth of 1 mm as flow paths; holes (7-21) of 2 mm in diameter as flow paths; and elliptic grooves (7-25) of a depth of 1 mm as trap parts. A base plate (7-3) shown in FIG. 7-c was made with a polycarbonate plate of 120 mm in length, 150 mm in width and a thickness of 10 mm. Into the base plate were worked holes (7-32) of 2 mm in diameter penetrable by a 2-mm stainless steel pan-head screw used in crimping; holes (7-34) of 2 mm in diameter having a clearance such that a rod-shaped element (7-8) is movable when inserted therein; grooves (7-33) of a width of 1 mm and a depth of 1 mm as flow paths; holes (7-31) of 2 mm in diameter as flow paths; a pentangular hole (7-36) functioning as a reaction chamber; and a hole (7-36) of 5 mm in diameter as a sample introduction opening to the reaction chamber. Further, a 6-mm female screw hole was worked into the upper part of an open part (7-38) of the sample introduction opening. A base plate (7-4) shown in FIG. 7-d was made with a polycarbonate plate of 120 mm in length, 150 mm in width and a thickness of 2 mm. Into the base plate were worked holes (7-42) of 2 mm in diameter penetrable by a 2-mm stainless steel pan-head screw used in crimping; holes (7-44) of 2 mm in diameter having a clearance such that a rod-shaped element (7-8) is movable when inserted therein; and grooves (7-43) of a width of 1 mm and a depth of 1 mm as flow paths. A sealing plate (7-5) shown in FIG. 7-e was made with a silicon rubber plate of 70 mm in length, 150 mm in width and a thickness of 1 mm. Into the sealing plate were worked holes (7-51) of 2 mm in diameter as flow paths; holes (7-52) of 2 mm in diameter penetrable by a 2-mm stainless steel pan-head screw used in crimping; holes (7-54) of 2 mm in diameter having a clearance such that a rod-shaped element (7-8) is movable when inserted therein, and provided such that when a part of the rod-shaped element having a diameter of 2 mm is inserted therein hermeticity is maintained; and elliptic holes (7-55) provided such that two cylindrical magnets of 5 mm in diameter and 4 mm in length for a trap can be aligned and inserted therein. A sealing plate (7-6) shown in FIG. 7-f was made with a silicon rubber plate of 120 mm in length, 150 mm in width and a thickness of 1 mm. Into the sealing plate were worked holes (7-61) of 2 mm in diameter as flow paths; holes (7-62) of 2 mm in diameter penetrable by a 2-mm stainless steel pan-head screw used in crimping; holes (7-64) of 2 mm in diameter having a clearance such that a rod-shaped element (7-8) is movable when inserted therein, and provided such that when a part of the rod-shaped element having a diameter of 2 mm is inserted therein hermeticity is maintained; elliptic holes (7-65) in positions corresponding to the positions of elliptic grooves (7-25) of base plate 7-2 as a trap part; and a pentangular hole (7-66) functioning as a reaction chamber. A sealing plate (7-7) shown in FIG. 7-g was made with a silicon rubber plate of 120 mm in length, 150 mm in width and a thickness of 1 mm. Into the sealing plate were worked holes (7-72) of 2 mm in diameter penetrable by a 2-mm stainless steel pan-head screw used in crimping; holes (7-74) of 2 mm in diameter having a clearance such that a rod-shaped element (7-8) is movable when inserted therein, and provided such that when a part of the rod-shaped element having a diameter of 2 mm is inserted therein hermeticity is maintained; and a pentangular hole (7-76) functioning as a reaction chamber. A rod-shaped element (7-8) shown in FIG. 7-h was made with a stainless steel rod of 2 mm in diameter and 27 mm in length. The rod-shaped element was processed such that a midsection (7-81) of the rod had a width of 2 mm across in the lengthwise direction and a diameter of 1 mm. These parts were crimped in a multilayered shape using a set of a stainless steel pan-head screw (diameter 2 mm, length 20 mm) and nut, to thus construct the cartridge shown in FIG. 7-i.

To the above cartridge were respectively mounted a plastic syringe containing 900 μl of a solution for adsorbing/releasing nucleic acid (20 mM EDTA, 1.3% Triton™ X-100, 50 mM Tris/HCl buffer solution comprising 5.25 M guanidine thiocyanate, pH 6.4) as reservoir 7-R1; a plastic syringe containing 1000 μl of washing liquid (70% ethanol) as reservoir 7-R2; a plastic syringe containing 1000 μl of washing liquid (99% ethanol) as reservoir 7-R3; and a plastic syringe containing 500 μl of eluate (distilled water) as reservoir 7-R4. The cartridge was provided such that a rod-shaped element (7-8) installed at each valve (7-V1 to 7-V11) is moved by a pressing force from outside, thereby enabling opening and closing of a flow path of a valve part. In addition, mounting and removal of magnets (two cylindrical magnets of 5 mm in diameter and 4 mm in length) at the trap parts (7-M1 to 7-M3) is also enabled. Further, using a connector of a tube used in liquid chromatography, a Teflon™ tube (inner diameter 0.9 mm, outer diameter 1.7 mm) was connected to each connection opening (7-P1 to 7-P3) with a pump part, and furthermore, at the end of each tube, a pump (diverted from P-6000, Pharmacia) that supplies air was connected, and by this air supply, feeding of fluid is enabled. Furthermore, in a cartridge lower part containing a reaction chamber (7-R5), heating with a heater (7-H1) by means of a hot water bath is enabled.

Using this cartridge, nucleic acid was extracted and purified from Escherichia coli by the following procedure. First, all the valves (7-V1 to 7-V11) were closed, and magnets were mounted at the trap parts (7-M1 to 7-M3). Next, Escherichia Coli (10⁷ cells) and 40 μl of magnetic silica particle suspension were inserted into the reaction chamber (7-R5). After introduction of the bacteria, a sample introduction opening (7-S1) of the upper part of the reaction chamber (7-R5) was closed using a connector of a tube used in liquid chromatography having one end closed. The lower part (7-H1) of the cartridge containing the reaction chamber (7-R5) was heated to 98° C. in a hot water bath. After opening valve 7-V3, a solution for adsorbing/releasing nucleic acid of reservoir 7-R1 was introduced into reaction chamber 7-R5. After 3 minutes, valves 7-V1, 7-V4 and 7-V6 were opened, valve 7-V3 was close was supplied from connection opening 7-P1 with the pump, and liquid in the reaction chamber (7-R5) was discharged to a waste fluid outlet (7-W1). At this stage, magnetic silica particles were accumulated at the trap 7-M1 part. After closing valve 7-V1 and valve 7-V6, opening valve 7-V2, valve 7-V7, valve 7-V8 and valve 7-V10, and removing the magnets at the trap 7-M1 part, washing liquid of reservoir 7-R2 was flowed. Next, valve 7-V2 was opened, air was supplied from connection opening 7-P2 with the pump, fluid remaining in the reaction chamber (7-R5) was discharged to a waste fluid outlet 7-W2, and at the same time, magnetic silica particles were dispersed and washed and accumulated again at the trap 7-M2 part. Valve 7-V4 was closed, and magnets at the trap 7-M2 part were removed. Washing liquid of reservoir 7-R3 was flowed and discharged to waste fluid outlet 7-W2, and at the same time, magnetic silica particles were dispersed and washed and accumulated again at the trap 7-M3 part. Valve 7-V8 was closed, valve 7-V5 was opened, and air was supplied from air pump connection opening (7-P3) for 10 min at a rate of 50 ml/min, thus drying ethanol which remained on the magnetic silica particles. Valve 7-V5 and valve 7-V10 were closed, valve 7-V9 and valve 7-V11 were opened, and magnets at the trap 7-M3 part were removed. Eluate of reservoir 7-R4 was flowed, and while dispersing magnetic silica particles, eluate was recovered from recovery outlet 7-W3. Finally, using 5 μl of the recovered eluate, in the same manner as in Example 1, polymerase chain reaction was conducted, followed by electrophoresis and detection.

As shown in FIG. 7-j, the results showed that in almost the same position as 200 bp of a DNA molecular weight marker (lane 1), DNA amplified by this polymerase chain reaction was detected as a band (lane 2) of a size to be expected from the nucleotide sequence. Thus it was shown that DNA of Escherichia coli was isolated by the cartridge of the present invention.

### EXAMPLE 8

A base plate (8-1) shown in FIG. 8-a was made with a polycarbonate plate of 70 mm in length, 150 mm in width and a thickness of 5 mm. Into the base plate were worked 6-mm female screw holes (8-011) provided such that a connector of a tube used in liquid chromatography can be inserted therein; holes (8-012) of 2 mm in diameter penetrable by a 2-mm stainless steel pan-head screw used in crimping; holes (8-013) of 4 mm in diameter provided such that a plastic syringe can be inserted therein; holes (8-014) of 2 mm in diameter having a clearance such that a rod-shaped element (7-8), the same as that used in Example 7, is movable when inserted therein; and a rectangular hole (8-015) having a clearance such that base plates 8-5 and 8-6 and sealing plates 8-10 and 8-11 can be inserted therein. A base plate (8-2) shown in FIG. 8-b was made with a polycarbonate plate of 120 mm in length, 150 mm in width and a thickness of 2 mm. Into the base plate were worked holes (8-022) of 2 mm in diameter penetrable by a 2-mm stainless steel pan-head screw used in crimping; grooves (8-023) of a width of 1 mm and a depth of 1 mm as flow paths; holes (8-024) of 2 mm in diameter having a clearance such that a rod-shaped element (7-8) is movable when inserted therein; and a rectangular hole (8-025) having a clearance such that base plates 8-5 and 8-6 and sealing plates 8-10 and 8-11 can be inserted therein. A base plate (8-3) shown in FIG. 8-c was made with a polycarbonate plate of 120 mm in length, 150 mm in width and a thickness of 10 mm. Into the base plate were worked holes (8-032) of 2 mm in diameter penetrable by a 2-mm stainless steel pan-head screw used in crimping; holes (8-031) of 2 mm in diameter as flow paths; grooves (8-033) of a width of 1 mm and a depth of 1 mm as flow paths; holes (8-034) of 2 mm in diameter having a clearance such that a rod-shaped element (7-8) is movable when inserted therein; a pentangular hole (8-036) functioning as a reaction chamber; and a hole (8-037) of 5 mm in diameter as a sample introduction opening to the reaction chamber. Further, a 6-mm female screw hole (8-038) was worked into the upper part of an open part of the sample introduction opening. A base plate (8-4) shown in FIG. 8-d was made with a polycarbonate plate of 120 mm in length, 150 mm in width and a thickness of 2 mm. Into the base plate were worked holes (8-042) of 2 mm in diameter penetrable by a 2-mm stainless steel pan-head screw used in crimping; holes (8-041) of 2 mm in diameter as flow paths; grooves (8-043) of a width of 1 mm and a depth of 1 mm as flow paths; and holes (8-044) of 2 mm in diameter having a clearance such that a rod-shaped element (7-8) is movable when inserted therein. A base plate (8-5) shown in FIG. 8-e was made with a polycarbonate plate of 20 mm in length, 30 mm in width and a thickness of 5 mm. Into the base plate were worked holes (8-052) of 2 mm in diameter penetrable by a 2-mm stainless steel pan-head screw used in crimping; holes (8-051) of 2 mm in diameter as flow paths; and a groove (8-053) of a width of 1 mm and a depth of 1 mm as a flow path. A base plate (8-6) shown in FIG. 8-f was made with a polycarbonate plate of 20 mm in length, 30 mm in width and a thickness of 2 mm. Into the base plate were worked holes (8-062) of 2 mm in diameter penetrable by a 2-mm stainless steel pan-head screw used in crimping; a hole (8-061) of 2 mm in diameter as a flow path; and, as a trap part, a hole (8-065) of 8 mm in diameter to which a silica membrane can be installed. A sealing plate (8-7) shown in FIG. 8-g was made with a silicon rubber plate of 70 mm in length, 150 mm in width and a thickness of 1 mm. Into the sealing plate were worked holes (8-072) of 2 mm in diameter penetrable by a 2-mm stainless steel pan-head screw used in crimping; holes (8-074) of 2 mm in diameter having a clearance such that a rod-shaped element (7-8) is movable when inserted therein, and provided such that when a part of the rod-shaped element having a diameter of 2 mm is inserted therein hermeticity is maintained; a rectangular hole (8-075) having a clearance such that base plates 8-5 and 8-6 and sealing plates 8-10 and 8-11 can be inserted therein; and holes (8-071 mm in diameter as flow paths. A sealing plate (8-8) shown in FIG. 8-h was made with a silicon rubber plate of 120 mm in length, 150 mm in width and a thickness of 1 mm. Into the sealing plate were worked holes (8-082) of 2 mm in diameter penetrable by a 2-mm stainless steel pan-head screw used in crimping; holes (8-084) of 2 mm in diameter having a clearance such that a rod-shaped element (7-8) is movable when inserted therein, and provided such that when a part of the rod-shaped element having a diameter of 2 mm is inserted therein hermeticity is maintained; a rectangular hole (8-085) having a clearance such that base plates 8-5 and 8-6 and sealing plates 8-10 and 8-11 can be inserted therein; a pentangular hole (8-086) functioning as a reaction chamber; and holes (8-081) of 2 mm in diameter as flow paths. A sealing plate (8-9) shown in FIG. 8-i was made with a silicon rubber plate of 120 mm in length, 150 mm in width and a thickness of 1 mm. Into the sealing plate were worked holes (8-092) of 2 mm in diameter penetrable by a 2-mm stainless steel pan-head screw used in crimping; holes (8-094) of 2 mm in diameter having a clearance such that a rod-shaped element (7-8) is movable when inserted therein, and provided such that when a part of the rod-shaped element having a diameter of 2 mm is inserted therein hermeticity is maintained; a pentangular hole (8-096) functioning as a reaction chamber; and holes (8-091) of 2 mm in diameter as flow paths. A sealing plate (8-10) shown in FIG. 8-j was made with a silicon rubber plate of 20 mm in length, 30 mm in width and a thickness of 1 mm. Into the sealing plate were worked holes (8-102) of 2 mm in diameter penetrable by a 2-mm stainless steel pan-head screw used in crimping, and holes (8-101) of 2 mm in diameter as flow paths. A sealing plate (8-11 ) shown in FIG. 8-k was made with a silicon rubber plate of 20 mm in length, 30 mm in width and a thickness of 1 mm. Into the sealing plate were worked holes (8-112) of 2 mm in diameter penetrable by a 2-mm stainless steel pan-head screw used in crimping; a hole (8-111) of 2 mm in diameter as a flow path; and, as a trap part, a hole (8-115) of 6 mm in diameter provided such that a silica membrane can be installed thereto and hermeticity of the flow path can be retained. These parts and a rod-shaped element (7-8), the same as that used in Example 7, were assembled with a set of stainless steel pan-head screws (diameter 2 mm, length 25 mm) and nuts to construct the cartridge shown in FIG. 8-1.

To the above cartridge were respectively mounted a plastic syringe containing 900 μl of a solution for adsorbing/releasing nucleic acid (20 mM EDTA, 1.3% Triton™ X-100, 50 mM Tris/HCl buffer solution comprising 5.25 M guanidine thiocyanate, pH 6.4) as reservoir 8-R11; a plastic syringe containing 1000 μl of washing liquid (70% ethanol) as reservoir 8-R12; a plastic syringe containing 1000 μl of washing liquid (99% ethanol) as reservoir 8-R13, and a plastic syringe containing 500 μl of eluate (distilled water) as reservoir 8-R14. Moreover, a silica membrane (one taken from DNeasy™ Tissue Kit, Qiagen, and used, diameter approximately 8 mm, thickness approximately 2 mm) was installed at the part of trap 8-M11. The cartridge was provided such that, a rod-shaped element (7-8) installed at each valve (8-V21 to 8-V32) is moved by means of a pressing force from outside, thereby enabling opening and closing of a flow path of a valve part. Further, using a connector of a tube used in liquid chromatography, a Teflon™ tube (inner diameter 0.9 mm, outer diameter 1.7 mm) was connected to each connection opening (8-P11, 8-P12) with a pump part, and moreover, at the end of each tube a pump (diverted from P-6000, Pharmacia) that supplies air was connected, and by such air supply, feeding of fluid is enabled. Furthermore, in a cartridge lower part containing a reaction chamber (8-R15), heating with a heater (8-H11) by means of a hot water bath is enabled.

Using this cartridge, nucleic acid of bacteria was extracted and purified from Escherichia coli by the following procedure. First, all the valves (8-V21 to 8-V32) were closed. Next, Escherichia Coli (10⁷ cells) were inserted into the reaction chamber (8-R15), and a sample introduction opening (8-S11) of the upper part of the reaction chamber was closed using a nut of 6 mm in diameter and 10 mm in length. The lower part (8-H11) of the cartridge containing the reaction chamber (8-R15) was heated to 98° C. in a hot water bath. After opening valves 8-V22 and 8-26, a solution for adsorbing/releasing nucleic acid of reservoir 8-R11 was introduced into reaction chamber 8-R15. After 3 minutes, valves 8-V21, 8-V27 and 8-V32 were opened, valve 8-V26 was closed, air was supplied from connection opening 8-P11 with the pump, and liquid in reaction chamber 8-R15 was discharged to waste fluid outlet 8-W11. At this stage, nucleic acid released from Escherichia coli was adsorbed to the silica membrane at trap 8-M11. Valve 8-V22 was closed and valve 8-V24 was opened, and washing liquid of reservoir 8-R12 was flowed. Further, valve 8-V23 was opened, air was supplied from connection opening 8-P11 with the pump, and fluid remaining in reaction chamber 8-R15 was discharged to waste fluid outlet 8-W11. Valve 8-V27 was closed, valve 8-V28 was opened, and washing liquid of reservoir 8-R13 was flowed. Then, valve 8-V25 was opened and air was supplied from connection opening 8-P11 with the pump to discharge fluid in the flow path to waste fluid outlet 8-W11. Valve 8-V28 was closed, valve 8-V30 was opened, and air was supplied from air pump connection opening 8-P12 for 10 minutes at a flow rate of 50 ml/min, thus drying ethanol which remained on the silica membrane. Valve 8-V30 and valve 8-V32 were closed, valve 8-V29 and valve 8-V31 were opened, eluate of reservoir 8-R14 was flowed, and eluate was recovered from recovery outlet 8-W12. Finally, using 5 μl of the recovered eluate, in the same manner as in Example 1, polymerase chain reaction was conducted, followed by electrophoresis and detection.

As shown in FIG. 8-m, the results showed that in almost the same position as 200 bp of a DNA molecular weight marker (lane 1), DNA amplified by this polymerase chain reaction was detected as a band (lane 2) of a size to be expected from the nucleotide sequence. Thus, it was shown that DNA of Escherichia coli was isolated by the cartridge of the present invention.

### EXAMPLE 9

A base plate (9-1) shown in FIG. 9-a was made with a polycarbonate plate of 20 mm in length, 20 mm in width and a thickness of 5 mm. Into the base plate were worked a 6-mm female screw hole (9-12) provided such that a connector of a tube used in liquid chromatography can be inserted therein, and holes (9-13) of 2 mm in diameter penetrable by a 2-mm stainless steel pan-head screw used in crimping. A base plate (9-2) shown in FIG. 9-b was made using a polycarbonate plate of 150 mm in length, 155 mm in width and a thickness of 2 mm. Into the base plate were worked holes (9-21) of 2 mm in diameter penetrable by a 2-mm stainless steel pan-head screw used in crimping; holes (9-22) of 1.6 mm in diameter penetrable by a 1.6-mm stainless steel pan-head screw used in crimping; a groove (9-23) of a width of 0.5 mm and a depth of 1 mm as a flow path; and a hole (9-24) of 1 mm in diameter as a flow path. A base plate (9-3) shown in FIG. 9-c was made with a polycarbonate plate of 150 mm in length, 155 mm in width and a thickness of 2 mm. Into the base plate were worked holes (9-31) of 2 mm in diameter penetrable by a 2-mm stainless steel pan-head screw used in crimping; holes (9-32) of 1.6 mm in diameter penetrable by a 1.6-mm stainless steel pan-head screw used in crimping; a groove (9-33) of a width of 0.5 mm and a depth of 1 mm as a flow path; and a hole (9-34) of 1 mm in diameter as a flow path. A sealing plate (9-4) shown in FIG. 9-d was made with a Teflon™ plate of 20 mm in length, 20 mm in width and a thickness of 1 mm. Into the sealing plate were worked holes (9-41) of 2 mm in diameter penetrable by a 2-mm stainless steel pan-head screw used in crimping, and a hole (9-42) of 2 mm in diameter as a flow path. A sealing plate (9-5) shown in FIG. 9-e was made with a Teflon™ M plate of 150 mm in length, 155 mm in width and a thickness of 1 mm. Into the sealing plate were worked holes (9-51) of 2 mm in diameter penetrable by a 2-mm stainless steel pan-head screw used in crimping; holes (9-52) of 1.6 mm in diameter penetrable by a 1.6-mm stainless steel pan-head screw used in crimping; and a hole (9-53) of 1 mm in diameter as a flow path. Using these parts and sets of screws (diameter 2 mm, length 20 mm; and diameter 1.6 mm, length 8 mm) and nuts (M2, M1.6), the cartridge shown in FIG. 9-f was assembled. Next, to enable a reciprocal reaction between two differing temperatures that takes place in a polymerase chain reaction to be conducted 40 times, two of the cartridges shown in FIG. 9-f were combined, and furthermore, a connector (9-71) of a tube used in liquid chromatography, a Teflon™ tube (inner diameter 0.5 mm, outer diameter 1.7 mm) (9-72), a two-way flow valve (Pharmacia) (9-73), a pump (P-500, Pharmacia) (9-74), and a syringe (9-75) were connected thereto, thereby constructing the apparatus shown in FIG. 9-g. Heating of the cartridge part to the two temperatures of 92° C. and 65° C. was enabled by means of heater (9-76, 9-77).

Escherichia coli-derived nucleic acid was purified by the method described in Example 1. To 50 μl of the purified Escherichia coli DNA solution were added 10 μl each of 2 types of 20 μM oligonucleotide (SEQ ID NO: 1, SEQ ID NO: 2) solution, 20 μl of substrate solution, 25 μl of DNA polymerase solution, and 50 μl of buffer solution (2.5W/V % BSA, 0.5% Triton™ X-100, 300 mM Tris/HCl, 17.5 mM magnesium chloride, pH 9.5), and distilled water was further added thereto to bring the total volume to 250 μl. This sample was poured into the syringe (9-75). By previously filling the inside of a flow path of the cartridge with mineral oil using the pump (9-74), the inner wall of the inside of the flow path was coated with mineral oil. Next, after rotating the valve (9-73), the sample in the syringe (9-75) was introduced into the cartridge. Then the valve (9-73) was rotated, mineral oil was again fed into the cartridge from the pump (9-74) at a flow rate of 9 ml/hr and PCR reaction conducted, and reaction solution was recovered from a recovery outlet (9-78). By the same method as shown in Example 1, electrophoresis and detection were performed using 10 μl of the eluate collected from the recovery outlet.

As shown in FIG. 9-h, the results showed that in almost the same position as 200 bp of a DNA molecular weight marker (lane 1), DNA (lane 2) amplified by this polymerase chain reaction was detected as a band of a size to be expected from the nucleotide sequence. Thus, using the technique of the present invention, an amplification reaction of nucleic acid was accomplished.

### Reference Example 3

For the cartridge used in Example 9, the following experiment was conducted using 250 μl of a blue colored aqueous solution in the syringe (9-75) instead of the sample solution. First, as in Example 9, by previously filling the inside of a flow path of the cartridge with mineral oil using the pump (9-74), the inner wall of the inside of the flow path was coated with mineral oil. Next, after rotating the valve (9-73), the sample in the syringe (9-75) was introduced into the cartridge. Then the valve (9-73) was rotated, mineral oil was again fed into the cartridge from the pump (9-74) at a flow rate of 9 ml/hr, and reaction solution was recovered from the recovery outlet (9-78). As a result, about 200 μl of colored aqueous solution was recovered, and no change was observed in the concentration of coloring liquid. In contrast, feeding of fluid was performed in a similar manner, but with distilled water being fed from the pump (9-74) in place of mineral oil. As a result, liquid from the recovery outlet was recovered in a state in which it had been diluted by the distilled water fed from the pump, and by observation with the unaided eye it was estimated that the liquid had been diluted to a volume of approximately 1 ml.

### EXAMPLE 10

A base plate (10-1) shown in FIG. 10-a was made with a polycarbonate plate of 42.5 mm in length, 200 mm in width and a thickness of 5 mm. Into the base plate were worked 6-mm female screw holes (10-11) provided such that a connector of a tube used in liquid chromatography can be inserted therein; holes (10-12) of 2 mm in diameter penetrable by a 2-mm stainless steel pan-head screw used in crimping; and holes (10-13) of 2 mm in diameter having a clearance such that a rod-shaped element (7-8), the same as that used in Example 7, is movable when inserted therein. A base plate (10-2) shown in FIG. 10-b was made with a polycarbonate plate of 90 mm in length, 200 mm in width and a thickness of 2 mm. Into the base plate were worked holes (10-21) of 2 mm in diameter as flow paths; holes (10-22) of 2 mm in diameter penetrable by a 2-mm stainless steel pan-head screw used in crimping; holes (10-23) of 1.6 mm in diameter penetrable by a 1.6-mm stainless steel pan-head screw used in crimping; holes (10-24) of 2 mm in diameter having a clearance such that a rod-shaped element (7-8) is movable when inserted therein; and grooves (10-25) of a width of 1 mm and a depth of 1 mm as flow paths. A base plate (10-3) shown in FIG. 10-c was made using a polycarbonate plate of 90 mm in length, 200 mm in width and a thickness of 2 mm. Into the base plate were worked holes (10-31) of 2 mm in diameter as flow paths; holes (10-32) of 2 mm in diameter penetrable by a 2-mm stainless steel pan-head screw used in crimping; holes (10-33) of 1.6 mm in diameter penetrable by a 1.6-mm stainless steel pan-head screw used in crimping; holes (10-34) of 2 mm in diameter having a clearance such that a rod-shaped element (7-8) is movable when inserted therein; and grooves (10-35) of a width of 1 mm and a depth of 1 mm as flow paths. A base plate (10-4) shown in FIG. 10-d was made using a polycarbonate plate of 57.5 mm in length, 200 mm in width and a thickness of 2 mm. Into the base plate were worked holes (10-41) of 2 mm in diameter penetrable by a 2-mm stainless steel pan-head screw used in crimping; holes (10-42) of 1.6 mm in diameter penetrable by a 1.6-mm stainless steel pan-head screw used in crimping; holes (10-43) of 2 mm in diameter having a clearance such that a rod-shaped element (7-8) is movable when inserted therein; and grooves (10-44) of a width of 1 mm and a depth of 1 mm as flow paths. A sealing plate (10-5) shown in FIG. 10-e was made using a Teflon™ plate of 42.5 mm in length, 200 mm in width and a thickness of 1 mm. Into the sealing plate were worked holes (10-51) of 2 mm in diameter as flow paths; holes (10-52) of 2 mm in diameter penetrable by a 2-mm stainless steel pan-head screw used in crimping; and holes (10-53) of 2 mm in diameter having a clearance such that a rod-shaped element (7-8) is movable when inserted therein. A sealing plate (10-6) shown in FIG. 10-f was made using a Teflon™ plate of 90 mm in length, 200 mm in width and a thickness of 1 mm. Into the sealing plate were worked holes (10-61) of 2 mm in diameter as flow paths; holes (10-62) of 2 mm in diameter penetrable by a 2-mm stainless steel pan-head screw used in crimping; and holes (10-63) of 2 mm in diameter having a clearance such that a rod-shaped element (7-8) is movable when inserted therein. A sealing plate (10-7) shown in FIG. 10-g was made using a Teflon™ plate of 57.5 mm in length, 200 mm in width and a thickness of 1 mm. Into the sealing plate were worked holes (10-71) of 2 mm in diameter as flow paths; holes (10-72) of 2 mm in diameter penetrable by a 2-mm stainless steel pan-head screw used in crimping; and holes (10-73) of 2 mm in diameter having a clearance such that a rod-shaped element (7-8) is movable when inserted therein.

Using these parts and a rod-shaped element (7-8), which was the same as that used in Example 7, and respective sets (10-81, 10-82) of pan-head screws (diameter 2 mm, length 20 mm; and diameter 1.6 mm, length 8 mm) and nuts (M2, M1.6), the cartridge shown in FIG. 10-h was assembled. To the cartridge in FIG. 10-h were further connected a connector (10-91) of a tube used in liquid chromatography, a Teflon tube (inner diameter 0.5 mm, outer diameter 1.7 mm) (10-92), two-way flow valves (Pharmacia) (10-93, 10-94), a pump (P-500, Pharmacia) (10-95), and a syringe (10-96), to thereby construct the apparatus shown in FIG. 10-i. Heating of the cartridge part to the two temperatures of 92° C. and 65° C. was enabled by means of heater (10-97, 10-98).

Previously, mineral oil was fed from the pump (10-95), and by opening and closing valve 10-99 and valve 10-100 and operation of the two-way flow valves (10-93, 10-94), the inside of the flow path of the cartridge was filled with the mineral oil to coat a inner wall inside the flow path with mineral oil. To 50 μl of Escherichia coli DNA solution purified in the same manner as in Example 1 were added 10 μl each of 2 types of 20 μM oligonucleotide (SEQ ID NO: 1, SEQ ID NO: 2), 20 μl of substrate solution, 25 μl of DNA polymerase solution, and 50 μl of buffer solution (2.5W/V % BSA, 0.5% Triton™ X-100, 300 mM Tris/HCl, 17.5 mM magnesium chloride, pH 9.5), and distilled water was further added thereto to bring the total volume to 250 μl. This sample was poured into the syringe (10-96).

Valves 10-99 and 10-100 were opened, and after introducing the sample in the syringe (10-96) into the cartridge from a tube connection opening (10-103), valves 10-99 and 10-100 were closed. The cartridge parts were heated to 95° C. and 65° C., respectively, by the heater (10-97, 10-98). Next, by combining changes of direction of the two-way flow valves (10-93, 10-94) and commencement and termination of feeding (1 ml/min) of mineral oil by the pump (10-95), operation was performed for 40 cycles wherein one cycle comprised the four steps of 1: feeding at 1 ml/min from pump connection opening (10-101) and discharge from pump connection opening (10-102) for 6 seconds; 2: cessation for 12 seconds; 3: feeding at 1 ml/min from pump connection opening (10-102) and discharge from pump connection opening (10-101) for 6 seconds; and 4: cessation for 12 seconds. By this operation, a sample solution was flowed back and forth 40 times through the 95° C. part and the 65° C. part within the cartridge, and as a result, a polymerase chain reaction was achieved. After reaction, valve 10-100 was opened, feeding of solution was performed at a flow rate of 1 ml/min from pump connection opening 10-101 and pump connection opening 10-102, and reaction solution was recovered in a container (10-105) from a recovery outlet (10-104). Using the recovered reaction solution, electrophoresis and detection were performed in the same manner as in Example 1.

As shown in FIG. 10-j, the results showed that in almost the same position as 200 bp of a DNA molecular weight marker (lane 1), DNA (lane 2) amplified by this polymerase chain reaction was detected as a band of a size to be expected from the nucleotide sequence. Thus it was confirmed that an amplification reaction of nucleic acid was achieved using the technique of the present invention.

According to the present invention, it is possible to provide a method for isolating nucleic acid wherein there are few inhibitors when performing amplification reaction in a solution of nucleic acid isolated from a material containing nucleic acid, even for nucleic acid of very small trace amounts. Therefore, detection is simplified when detecting the presence of a specific microorganism in a specimen or when conducting-gene diagnosis of human, or the like, and the invention is therefore useful. Moreover, according to the present invention it is possible to easily provide a cartridge that performs various chemical reactions. Further, according to the present invention, it is possible to construct a cartridge for nucleic acid isolation that applies the above method for isolating nucleic acid. Using a cartridge for chemical reaction and a simple mechanism that controls chemical reaction on the cartridge according to the present invention, analysis in various fields can be performed conveniently, quickly and safely. Further, using the cartridge for nucleic acid isolation and a simple mechanism that controls reaction on the cartridge according to the present invention, gene diagnosis in the field of medical treatment can be performed conveniently, quickly and safely.

What is claimed is:

1. A method for isolating nucleic acid, comprising the steps of:
1) mixing a material containing nucleic acid and a solution for adsorbing/releasing nucleic acid, and contacting a mixture thereof with a nucleic acid-binding carrier to prepare a nucleic acid-adsorbed nucleic acid-binding carrier;
2) separating the nucleic acid-adsorbed nucleic acid-binding carrier;
3) washing the nucleic acid-adsorbed nucleic acid-binding carrier with washing liquid;
4) drying the nucleic acid-adsorbed nucleic acid-binding carrier; and
5) eluting nucleic acid from the nucleic acid-binding carrier with eluate;
wherein the step 1) is conducted under heating.

2. A method for isolating nucleic acid, comprising the steps of:
1) mixing a material containing nucleic acid, a nucleic acid-binding magnetic carrier, and a solution for adsorbing/releasing nucleic acid to prepare a suspension including the carrier to which nucleic acid has bound;
2) separating the carrier to which nucleic acid has bound from the liquid phase of the suspension;
3) washing the carrier to which nucleic acid has bound with washing liquid;
4) drying the carrier to which nucleic acid has bound; and
5) eluting nucleic acid from the carrier with eluate;
wherein step 1) is conducted under heating and, in steps 2) to 4), a flow path for nucleic acid isolation is provided along which a magnetic field capable of retaining the carrier can be applied in at least two places, and when suspension containing the carrier to which nucleic acid has bound is flowed in the flow path, the magnetic field is applied at one place of the at least two places and the carrier is separated from the suspension, and when at least one solution for washing is flowed in the flow path, application of the magnetic field at a place where the carrier is retained is released, and by applying the magnetic field at a place downstream of the place the carrier was retained, the carrier is washed and separated from the solution for washing, and further, a solution for eluting nucleic acid is flowed in the flow path to elute nucleic acid from the carrier.

3. The method for isolating nucleic acid of claim 1 or 2, wherein heating of step 1) is conducted at a temperature of 60° C. or higher and 130° C. or lower.

4. The method for isolating nucleic acid of claim 1 or 2, wherein heating of step 1) is conducted at a temperature of 80° C. or higher and 110° C. or lower.

5. The method for isolating nucleic acid of claim 1 or 2, wherein heating of step 1) is conducted at a temperature of 90° C. or higher and 100° C. or lower.

6. The method for isolating nucleic acid of claim 1 or 2, wherein heating of step 1) is conducted for 1 minute or more and 1 hour or less.

7. The method for isolating nucleic acid of claim 1 or 2, wherein the solution for adsorbing/releasing nucleic acid is a solution containing a high-chaotropic substance.

8. The method for isolating nucleic acid of claim 1, wherein the carrier is silica or a silica derivative.

9. The method for isolating nucleic acid of claim 8, wherein the carrier is a silica particle or a silica derivative particle.

10. The method for isolating nucleic acid of claim 8, wherein the carrier is a membrane consisting of silica or a silica derivative.

11. The method for isolating nucleic acid of claim 1 or 2, wherein the carrier is a magnetic silica particle or a magnetic silica derivative particle.

12. The method for isolating nucleic acid of claim 1 or 2, wherein the washing liquid is a solution containing ethanol.

13. The method for isolating nucleic acid of claim 12, wherein the washing liquid is a solution containing 70% or more ethanol.

14. The method for isolating nucleic acid of claim 2, wherein the step of washing the carrier and separating it from the washing liquid further comprises at least one of the steps of:
a) heating the downstream place to dry the carrier retained in the place, and
b) blowing air to the downstream place to dry the carrier retained in the place.

15. The method for isolating nucleic acid of claim 2, wherein the step of eluting nucleic acid from the carrier comprises the steps of: flowing the eluate in the flow path, releasing application of the magnetic field at the downstream place, and eluting nucleic acid from the carrier.

16. The method for isolating nucleic acid of claim 1 or 2, wherein the eluate comprises an enzyme, an oligonucleotide and a substrate for a nucleic acid amplification reaction.

17. A method for isolating and amplifying nucleic acid, wherein nucleic acid isolated by the method for isolating nucleic acid of claim 1 or 2 is further amplified by a nucleic acid amplification reaction.

18. The method for isolating and amplifying nucleic acid of claim 17, wherein the nucleic acid amplification reaction is conducted in a flow path communicating from the flow path for nucleic acid isolation.

19. The method for isolating and amplifying nucleic acid of claim 17, wherein the nucleic acid amplification reaction is a polymerase chain reaction (PCR).

20. A cartridge for chemical reaction, having at least one reservoir and/or reaction chamber and at least one flow path, and for applying a chemical reaction to a given ingredient contained in a liquid or gaseous sample or a liquid or gaseous reagent, or a mixed fluid of the sample and the reagent by flowing the sample or the reagent, or the mixed fluid from the at least one reservoir and/or reaction chamber into the at least one flow path, wherein the cartridge feeds a sample solution or a reagent solution or a mixed solution of the sample and the reagent into the flow path using a feeding liquid that is immiscible with and is phase separated from the solution.

21. A cartridge for chemical reaction, having at least one reservoir and/or reaction chamber and at least one flow path, and for applying a chemical reaction to a given ingredient contained in a liquid or gaseous sample or a liquid or gaseous reagent, or a mixed fluid of the sample and the reagent by flowing the sample or the reagent or the mixed fluid from the at least one reservoir and/or reaction chamber into the at least one flow path,
wherein the cartridge has a multilayered structure of three or more layers in which at least one of a tabular member for hermeticity comprising an elastomer and at least two of a tabular member for a base plate comprising material having a lower elasticity and a higher degree of hardness than the elastomer are alternately interposed and crimped;
wherein the flow path comprises at least one member selected from the group consisting of: a groove and/or a hole provided in the tabular member for a base plate, a groove and/or a hole provided in the tabular member for hermeticity, and an aperture formed by transformation of a part of the tabular member for hermeticity due to pressure of the sample, the reagent or the mixed fluid; and
the reservoir and/or reaction chamber comprises a groove and/or a hole provided in the tabular member for hermeticity and/or the tabular member for a base plate.

22. The cartridge for chemical reaction of claim 21, wherein the chemical reaction is conducted by feeding a sample solution or a reagent solution or a mixed solution of the sample and the reagent into the flow path using a feeding liquid that is immiscible with and is phase separated from the solution.

23. The cartridge for chemical reaction of claim 21, wherein the cartridge comprises at least one valve controlling opening and closing of the flow path.

24. The cartridge for chemical reaction of claim 23, wherein at least one of the valves is a valve controlling opening and closing of a flow path on the cartridge, having a rod-shaped element, movement of the rod-shaped element being possible with respect to a flow path on the cartridge, the rod-shaped element having an open part and a closed part, the open part being of a structure such that a projected area to the vertical plane with respect to a movement direction is smaller than that of the closed part, and by a movement of the rod-shaped element a flow path on the cartridge and an open part of the rod-shaped element communicate, thus opening the valve, and by a movement of the rod-shaped element a flow path on the cartridge is blocked by a closed part of the rod-shaped element, thus closing the valve.

25. The cartridge for chemical reaction of claim 23, wherein at least one of the valves is a valve controlling opening and closing of the flow path by controlling formation of an aperture formed by transformation of a part of a tabular member for hermeticity caused by pressure of the sample, the reagent and/or the mixed fluid.

26. The cartridge for chemical reaction of claim 23, wherein the cartridge comprises a control apparatus controlling opening and closing of at least one of the valves by an actuator.

27. The cartridge for chemical reaction of claim 20 or 21, wherein the temperature in the cartridge is controlled by heating or cooling at least one part of the cartridge.

28. The cartridge for chemical reaction of claim 27, wherein the temperature is controlled by heating and/or cooling at least two places to respectively different temperatures.

29. The cartridge for chemical reaction of claim 28, wherein at least two places of the flow path are heated and/or cooled to control at respectively different temperatures, and the sample, the reagent or the mixed fluid is fed back and forth inside the flow path to apply a chemical reaction to a given ingredient in the sample, the reagent, or the mixed fluid.

30. The cartridge for chemical reaction of claim 20 or 21, wherein the chemical reaction comprises a nucleic acid amplification reaction.

31. The cartridge for chemical reaction of claim 30, wherein the nucleic acid amplification reaction is a polymerase chain reaction (PCR).

32. A cartridge for nucleic acid isolation, comprising at least one of:
a nucleic acid-binding carrier;
a solution for adsorbing/releasing nucleic acid that releases nucleic acid from a material containing nucleic acid and adsorbs it on the nucleic acid-binding carrier;
a washing liquid that washes a nucleic acid-binding carrier on which nucleic acid is adsorbed; and
an eluate that elutes nucleic acid from a nucleic acid-binding carrier on which nucleic acid is adsorbed.

33. A cartridge for nucleic acid isolation having the structure of the cartridge for chemical reaction of claim 20 or 21, which comprises at least one of:
a nucleic acid-binding carrier;
a solution for adsorbing/releasing nucleic acid that releases nucleic acid from a material containing nucleic acid and adsorbs it on the nucleic acid-binding carrier;
a washing liquid that washes a nucleic acid-binding carrier on which nucleic acid is adsorbed; and
an eluate that elutes nucleic acid from a nucleic acid-binding carrier on which nucleic acid is adsorbed.

34. The cartridge for nucleic acid isolation of claim 33, wherein the chemical reaction comprises a nucleic acid isolation reaction comprising the steps of:
1) mixing a material containing nucleic acid and a solution for adsorbing/releasing nucleic acid, and contacting a mixed solution thereof with a nucleic acid-binding carrier to prepare a nucleic acid-adsorbed nucleic acid-binding carrier;
2) separating the nucleic acid-adsorbed nucleic acid-binding carrier;
3) washing the nucleic acid-adsorbed nucleic acid-binding carrier;
4) drying the nucleic acid-adsorbed nucleic acid-binding carrier; and
5) eluting nucleic acid from the nucleic acid-binding carrier.

35. The cartridge for nucleic acid isolation of claim 34, wherein step 1) is conducted under heating.

36. The cartridge for nucleic acid isolation of claim 35, wherein heating of step 1) is conducted at a temperature of 60° C. or higher and 130° C. or lower.

37. The cartridge for nucleic acid isolation of claim 35, wherein heating of step 1) is conducted at a temperature of 80° C. or higher and 110° C. or lower.

38. The cartridge for nucleic acid isolation of claim 35, wherein heating of step 1) is conducted at a temperature of 90° C. or higher and 100° C. or lower.

39. The cartridge for nucleic acid isolation of claim 35, wherein heating of step 1) is conducted for 1 minute or more and 1 hour or less.

40. The cartridge for nucleic acid isolation of claim 32, wherein the solution for adsorbing/releasing nucleic acid is a solution containing a high-chaotropic substance.

41. The cartridge for nucleic acid isolation of claim 32, wherein the carrier is silica or a silica derivative.

42. The cartridge for nucleic acid isolation of claim 41, wherein the carrier is a silica particle or a silica derivative particle.

43. The cartridge for nucleic acid isolation of claim 41, wherein the carrier is a membrane consisting of silica or a silica derivative.

44. The cartridge for nucleic acid isolation of claim 32, wherein the carrier is a magnetic silica particle or a magnetic silica derivative particle.

45. The cartridge for nucleic acid isolation of claim 32, wherein the washing liquid is a solution containing ethanol.

46. The cartridge for nucleic acid isolation of claim 45, wherein the washing liquid is a solution containing 70% or more ethanol.

47. A cartridge for nucleic acid isolation, which is a cartridge for isolating nucleic acid from a nucleic acid-adsorbed nucleic acid-binding magnetic carrier, wherein the cartridge comprises a flow path for nucleic acid isolation, and wherein a magnetic field capable of retaining the carrier can be applied in at least two places along the flow path.

48. A cartridge for nucleic acid isolation, comprising:
a reaction chamber for mixing and reacting a material containing nucleic acid, a nucleic acid-binding magnetic carrier and a solution for adsorbing/releasing nucleic acid;
a flow path for nucleic acid isolation, wherein in at least two places along the flow path a magnetic field capable of retaining the carrier can be applied;
a reservoir for storing a solution for adsorbing/releasing nucleic acid;
a flow path linking the reservoir for storing a solution for adsorbing/releasing nucleic acid and the reaction chamber;
a flow path linking the reaction chamber and the flow path for nucleic acid isolation;
at least one reservoir for storing a solution for washing;
at least one flow path for washing which links the reservoir for storing a solution for washing and at least one member selected from the group consisting of: the reaction chamber, the flow path linking the reservoir for storing a solution for adsorbing/releasing nucleic acid and the reaction chamber, the flow path linking the reaction chamber and the flow path for nucleic acid isolation, and the flow path for nucleic acid isolation;
a reservoir for storing a solution for eluting nucleic acid; and
a flow path linking the reservoir for storing a solution for eluting nucleic acid and at least one member selected from the group consisting of: the reaction chamber, the flow path linking the reservoir for storing a solution for adsorbing/releasing nucleic acid and the reaction chamber, the flow path linking the reaction chamber and the flow path for nucleic acid isolation, the flow path for nucleic acid isolation, and the flow path for washing.

49. The cartridge for nucleic acid isolation of claim 32, wherein a nucleic acid amplification reaction that amplifies a nucleic acid isolated by a nucleic acid isolation reaction can also be performed.

50. The cartridge for nucleic acid isolation of claim 49, wherein a nucleic acid amplification reaction is a polymerase chain reaction (PCR).


SEQUENCE LISTING (4 sequences)

SEQ ID NO: 1 - DNA, 24 bp, Escherichia Coli
ctaacaagttcccggcaatcatct

SEQ ID NO: 2 - DNA, 23 bp, Escherichia Coli
tcgatgtgctgcagcttcggttt

SEQ ID NO: 3 - DNA, 24 bp, Bacillus subtilis
accttgacggtacctaaccagaaa

SEQ ID NO: 4 - DNA, 23 bp, Bacillus subtilis
gcatttcaccgctacacgtggaa